(19) United States
(12) Patent Application Publication
(10) Pub. No.: US 2007/0270366 A1
(43) Pub. Date: Nov. 22, 2007

(54) DOUBLE STRANDED NUCLEIC ACID MOLECULES TARGETED TO IL-4 RECEPTOR ALPHA

(76) Inventors: James G. Karras, San Marcos, CA (US); Susan Gregory, San Diego, CA (US); Andrew T. Watt, Oceanside, CA (US); Jeffrey R. Crosby, Carlsbad, CA (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/612,904

(22) Filed: Dec. 19, 2006

Related U.S. Application Data
(60) Provisional application No. 60/752,270, filed on Dec. 20, 2005.

(51) Int. Cl. A61K 48/00; C07H 21/00

(52) U.S. Cl. 514/44; 536/24.5

(57) ABSTRACT

Disclosed herein are compounds, compositions and methods for modulating the expression of IL-4R alpha in a cell, tissue or animal. Also provided are methods of target validation. Also provided are uses of disclosed compounds and compositions in the manufacture of a medicament for treatment of diseases and disorders.

### CROSS-REFERENCE TO RELATED APPLICATIONS

This application claims the benefit of priority to U.S. Provisional Application Ser. No. 60/752,270, filed Dec. 20, 2005, which is herein incorporated by reference in its entirety.

### INCORPORATION OF SEQUENCE LISTING

The present application is being filed along with a Sequence Listing in electronic format. The Sequence Listing is provided as a file entitled BIOL0075USSEQ.txt created Dec. 19, 2006, which is 137 Kb in size. The information in the electronic format of the sequence listing is incorporated herein by reference in its entirety.

### BACKGROUND

The cytokine IL-4 is produced by T helper type 2 (Th2) cells in response to antigen receptor engagement, and by mast cells and basophils upon cross-linkage of the high-affinity receptor for immunoglobulin E (IgE). Many of the responses elicited by the cytokine are associated with allergy, asthma, and inhibition of autoimmunity. The pleiotropic effects of the cytokine depend upon binding to and signaling through a receptor complex consisting of the IL-4R alpha chain (also known as IL-4Ra, CD124, and interleukin 4 receptor alpha chain) and a second transmembrane subunit (Kelly-Welch et al., Science, 2003, 300, 1527-1528; Nelms et al., Annu. Rev. Immunol., 1999, 17, 701-738).

IL-4 receptors are composed of two transmembrane proteins. The IL-4R alpha chain binds IL-4 with high affinity, leading to dimerization with another protein to form either a type I or a type II receptor. In cells with hematopoietic lineage, the type I receptor is formed by association of a common gamma chain, first identified as a component of the IL-2 receptor, with the IL-4R alpha chain. In nonhematopoietic cells, the type II receptor is formed by interaction of IL-4R alpha with IL-13R alpha1. Because both IL-4 receptor complexes require the IL-4R alpha chain for IL-4 mediated effects, this component is often simply equated with the IL-4 receptor.

A chemically modified IL-4R alpha antisense oligonucleotide (ASO) was identified that specifically inhibits IL-4R alpha protein expression in lung eosinophils, macrophages, dendritic cells, and airway epithelium following inhalation in allergen challenged mice (WO 2006/091841). Inhalation of IL-4R alpha ASO attenuated allergen-induced AHR, suppressed airway eosinophilia and neutrophilia, and inhibited production of airway Th2 cytokines and chemokines in previously allergen primed and challenged mice. Histological analysis of lungs from these animals demonstrated reduced goblet cell metaplasia and mucus staining that correlated with inhibition of Muc5AC gene expression in lung tissue. Therapeutic administration of inhaled IL-4R alpha ASO in chronically allergen challenged mice produced an anti-inflammatory spectrum of activity similar to that of systemically administered dexamethasone with the added benefit of reduced airway neutrophils.

Antisense oligonucleotides targeted to IL-4R alpha are described in WO 2006/091841 and in PCT/US2006/039168, filed Oct. 3, 2006, each of which are herein incorporated by reference.

A number of ASOs and siRNAs designed to target IL 4R-α have been reported for use as research or diagnostic tools, or as pharmaceuticals for the treatment of respiratory disease. US Patent Publication 2003-0104410 teaches an array of nucleic acid probes useful as research tools to identify or detect gene sequences. Allelic variations in the IL 4R-α gene have been identified that increase receptor signaling (Hershey et al., NEJM, 1997, 337:1720-1725; Rosa-Rosa et al., J. Allergy Clin. Immunol. 1999, 104:1008-1014; Kruse et al., Immunol., 1999, 96, 365-371). PCT Publication No. WO 2000/034789 teaches oligonucleotides for use in diagnostic testing to detect these allelic variations. PCT Publication WO 2002/085309 and WO 2004/011613 and US Patent Publication 2004-0049022 teach ASOs targeted to a series of genes potentially relevant to respiratory disease, including IL 4R-α, for use in pharmaceutical compositions. PCT Publication WO 2004/045543 teaches algorithms and rational design and selection of functional siRNAs including those targeted to IL 4R-α. Although it is suggested in these publications that the antisense compounds can be used in pharmaceutical compositions, there are no data demonstrating the efficacy of the compounds in vivo for the prevention, amelioration, and/or treatment of any disease or disorder.

IL-4R antisense oligonucleotides also are disclosed in U.S. Pat. No. 6,822,087 and Ikizawa et al. (Clin. Exp. Immunol., 1995, 100(3):380-382).

Double stranded siRNA molecules targeted to a variety of interleukins and interleukin receptors are taught in US Patent Publications 2005-0143333 and 2005-0261219.

Dreyfus et al. disclose the use of an external guide sequence targeting human IL-4R alpha mRNA (Dreyfus et al., Int. Immunopharmacol., 2004, 4, 1015-1027).

US Patent Publication 2004-0049022 relates to single or multiple target antisense oligonucleotides (STA or MTA oligos) of low or no adenosine content for respiratory disease-relevant genes, compositions thereof and methods for manufacturing the composition. The disclosure further relates to a method for screening candidate compounds useful for the prevention and/or treatment of respiratory diseases which bind to gene(s), EST(s), cDNA(s), mRNA(s), or their expressed product(s). Disclosed is a list of example nucleic acid targets including interleukin-4 receptor.

US Patent Publication 2004-0040052 is generally directed to a method of producing a transgenic cell comprising introducing into a cell a non-primate lentiviral expression vector comprising a nucleotide of interest (NOI). Also described is a method of producing a transgenic cell comprising introducing into a cell a lentiviral expression vector comprising a NOI capable of generating an antisense oligonucleotide, a ribozyme, an siRNA, a short hairpin RNA, a micro-RNA or a group 1 intron. Also described is a viral vector comprising a first nucleotide sequence, wherein said first nucleotide sequence comprises: (a) a second nucleotide sequence comprising an aptazyme; and (b) a third nucleotide sequence capable of generating a polynucleotide; wherein (a) and (b) are operably linked and wherein the aptazyme is activatable to cleave a transcript of the first nucleotide sequence such that said polynucleotide is generated. Disclosed is a list of genes that are associated with human disease, including IL4Ra.

US Patent Publication 2003-0078220 is directed to single nucleotide polymorphisms in the human Interleukin 4 Receptor Alpha (IL4R.alpha.) gene. Compositions and methods for detecting one or more of these polymorphisms are also disclosed, and various genotypes and haplotypes for the gene that exist in the population are described.

### SUMMARY

Provided herein are oligomeric compounds, especially nucleic acid and nucleic acid-like oligomers, which are targeted to a nucleic acid encoding IL-4R alpha. The compounds are preferably double stranded nucleic acid and nucleic-acid like oligomers. Most preferably compounds that are at least partially RNA or RNA-like. Further provided are antisense compounds which are oligomeric compounds that modulate the expression of IL-4R alpha. Also contemplated is a method of making an oligomeric compound comprising specifically hybridizing in vitro a first oligomeric strand comprising a sequence of at least 8 contiguous nucleobases of any of the sequences set forth in Tables 4, 5 and 7 to a second oligomeric strand comprising a sequence substantially complementary to said first strand.

Further provided are methods of modulating the expression of IL-4R alpha in cells, tissues or animals comprising contacting said cells, tissues or animals with one or more of the compounds or compositions provided herein. For example, in one embodiment, the compounds or compositions can be used to inhibit the expression of IL-4R alpha in cells, tissues or animals.

Further provided are methods of identifying the relationship between IL-4R alpha and a disease state, phenotype, or condition by detecting or modulating IL-4R alpha comprising contacting a sample, tissue, cell, or organism with one or more oligomeric compounds, measuring the nucleic acid or protein level of IL-4R alpha and/or a related phenotypic or chemical endpoint coincident with or at some time after treatment, and optionally comparing the measured value to a non-treated sample or sample treated with a further compound, wherein a change in said nucleic acid or protein level of IL-4R alpha coincident with said related phenotypic or chemical endpoint indicates the existence or presence of a predisposition to a disease state, phenotype, or condition.

Further provided are methods of screening for modulators of expression of IL-4R alpha by contacting a target segment of a nucleic acid molecule encoding IL-4R alpha with one or more candidate modulators, and selecting for one or more candidate modulators which decrease or increase the expression of a nucleic acid molecule encoding IL-4R alpha.

Further provided are methods of screening for additional modulators of expression of IL-4R alpha by contacting a validated target segment of a nucleic acid molecule encoding IL-4R alpha with one or more candidate modulators, and selecting for one or more candidate modulators which decrease or increase the expression of a nucleic acid molecule encoding IL-4R alpha.

Pharmaceutical, therapeutic and other compositions comprising the compounds described herein are also provided.

Also provided is the use of the compounds or compositions described herein in the manufacture of a medicament for the treatment of one or more conditions associated with IL-4R alpha. Further contemplated are methods where cells or tissues are contacted in vivo with an effective amount of one or more of the compounds or compositions provided herein. Also provided are ex vivo methods of treatment that include contacting cells or tissues with an effective amount of one or more of the compounds or compositions and then introducing said cells or tissues into an animal.

Further provided are double stranded antisense compounds wherein one strand is at least 70%, at least 80%, at least 90%, at least 95% or 100% complementary to a nucleic acid molecule encoding human IL-4R alpha. Also provided are double stranded antisense compounds wherein one strand is at least 70%, at least 80%, at least 90%, at least 95% or 100% identical to one of the illustrative antisense compounds provided herein. In addition, double stranded antisense compounds comprising at least one modification and compounds comprising a chimeric oligonucleotide are provided.

### DETAILED DESCRIPTION

Overview

Disclosed herein are oligomeric compounds, including antisense oligonucleotides and other antisense compounds for use in modulating the expression of nucleic acid molecules encoding IL-4R alpha. This is accomplished by providing oligomeric compounds which hybridize with one or more target nucleic acid molecules encoding IL-4R alpha. As used herein, the terms “target nucleic acid” and “nucleic acid molecule encoding IL-4R alpha” have been used for convenience to encompass DNA encoding IL-4R alpha, RNA (including pre-mRNA and mRNA or portions thereof) transcribed from such DNA, and also cDNA derived from such RNA.

The disclosure is not limited by the mechanism of action of the compounds provided herein. The principle behind antisense technology, including double stranded compounds that include an antisense strand targeted to a cellular RNA, is that an antisense compound, which hybridizes to a target nucleic acid, modulates gene expression activities such as transcription or translation. This sequence specificity makes antisense compounds extremely attractive as tools for target validation and gene functionalization, as well as therapeutics to selectively modulate the expression of genes involved in disease.

As shown herein, double stranded compounds targeted to human or mouse IL-4R alpha are capable of inhibiting expression of IL-4R alpha. Active double stranded compounds were shown to inhibit expression of IL-4R alpha in a dose-dependent manner. Furthermore, using mouse models of allergic inflammation, double stranded compounds targeted to IL-4R alpha were shown to reduce airway hyperresponsiveness, reduce Penh and reduce eosinophil recruitment to the lung. Thus, provided herein are double stranded antisense compounds effective for the treatment of airway hyperresponsiveness and pulmonary inflammation, which can be characteristics of asthma.

Antisense Mechanisms

Antisense mechanisms are all those involving the hybridization of a compound with target nucleic acid, wherein the outcome or effect of the hybridization is either target degradation or target occupancy with concomitant stalling of the cellular machinery involving, for example, transcription or splicing.

Target degradation can include an RNase H. RNase H is a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of DNA-like oligonucleotide-mediated inhibition of gene expression.

Target degradation can include RNA interference (RNAi). RNAi is a form of posttranscriptional gene silencing that was initially defined in the nematode, Caenorhabditis elegans, resulting from exposure to double-stranded RNA (dsRNA). In many species the introduction of double-stranded structures, such as double-stranded RNA (dsRNA) molecules, has been shown to induce potent and specific antisense-mediated reduction of the function of a gene or its associated gene products. The RNAi compounds are often referred to as short interfering RNAs or siRNAs. Recently, it has been shown that it is, in fact, the single-stranded RNA oligomers of antisense polarity of the siRNAs which are the potent inducers of RNAi (Tijsterman et al., Science, 2002, 295, 694-697).

Both RNAi compounds (i.e., single- or double-stranded RNA or RNA-like compounds) and single-stranded RNase H-dependent antisense compounds bind to their RNA target by base pairing (i.e., hybridization) and induce site-specific cleavage of the target RNA by specific RNAses; i.e., both are antisense mechanisms (Vickers et al., 2003, J. Biol. Chem., 278, 7108-7118). Double-stranded ribonucleases (dsRNases) such as those in the RNase III and ribonuclease L family of enzymes also play a role in RNA target degradation. Double-stranded ribonucleases and oligomeric compounds that trigger them are further described in U.S. Pat. Nos. 5,898,031 and 6,107,094.

Nonlimiting examples of an occupancy-based antisense mechanism whereby antisense compounds hybridize yet do not elicit cleavage of the target include inhibition of translation, modulation of splicing, modulation of poly(A) site selection and disruption of regulatory RNA structure. A method of controlling the behavior of a cell through modulation of the processing of an mRNA target by contacting the cell with an antisense compound acting via a non-cleavage event is disclosed in U.S. Pat. No. 6,210,892 and U.S. Pre-Grant Publication 20020049173. The references further teach antisense compounds targeted to a specific poly(A) site of mRNA that can be used to modulate the populations of alternatively polyadenylated transcripts and to disrupt RNA regulatory structure thereby affecting, for example, the stability of the targeted RNA and its subsequent expression.

Certain types of antisense compounds which specifically hybridize to the 5′ cap region of their target mRNA can interfere with translation of the target mRNA into protein. Such oligomers include peptide-nucleic acid (PNA) oligomers, morpholino oligomers and oligonucleosides (such as those having an MMI or amide internucleoside linkage) and oligonucleotides having modifications at the 2′ position of the sugar when such oligomers are targeted to the 5′ cap region of their target mRNA. This is believed to occur via interference with ribosome assembly on the target mRNA. Methods for inhibiting the translation of a selected capped target mRNA by contacting target mRNA with an antisense compound are disclosed in U.S. Pat. No. 5,789,573.

Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

In one embodiment, double-stranded antisense compounds encompass short interfering RNAs (siRNAs). As used herein, the term “siRNA” is defined as a double-stranded compound having a first and second strand and comprises a central complementary portion between said first and second strands and terminal portions that are optionally complementary between said first and second strands or with the target mRNA. The ends of the strands may be modified by the addition of one or more natural or modified nucleobases to form an overhang. In one nonlimiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand may contain modifications or additions to either terminus. For example, in one embodiment, both strands of the siRNA duplex would be complementary over the central nucleobases, each having overhangs at one or both termini. It is possible for one end of a duplex to be blunt and the other to have overhanging nucleobases. In one embodiment, the number of overhanging nucleobases is from 1 to 6 on the 3′ end of each strand of the duplex. In another embodiment, the number of overhanging nucleobases is from 1 to 6 on the 3′ end of only one strand of the duplex. In a further embodiment, the number of overhanging nucleobases is from 1 to 6 on one or both 5′ ends of the duplexed strands. In another embodiment, the number of overhanging nucleobases is zero.

In one embodiment, double-stranded antisense compounds are canonical siRNAs. As used herein, the term “canonical siRNA” is defined as a double-stranded oligomeric compound having a first strand and a second strand, each strand being 21 nucleobases in length, wherein the strands are complementary over 19 nucleobases and each strand has a deoxy thymidine dimer (dTdT) on the 3′ terminus, which in the double-stranded compound acts as a 3′ overhang.

Each strand of the siRNA duplex may be from about 8 to about 80, 10 to 50, 13 to 80, 13 to 50, 13 to 30, 13 to 24, 18 to 22, 19 to 23, 20 to 80, 20 to 50, 20 to 30, or 20 to 24 nucleobases. The central complementary portion may be from about 8 to about 80, 10 to 50, 13 to 80, 13 to 50, 13 to 30, 13 to 24, 18 to 22, 19 to 23, 20 to 80, 20 to 50, 20 to 30, or 20 to 24 nucleobases in length. The terminal portions can be from 1 to 6 nucleobases. The siRNAs may also have no terminal portions. The two strands of an siRNA can be linked internally leaving free 3′ or 5′ termini or can be linked to form a continuous hairpin structure or loop. The hairpin structure may contain an overhang on either the 5′ or 3′ terminus producing an extension of single-stranded character.

In another embodiment, the double-stranded antisense compounds are blunt-ended siRNAs. As used herein the term “blunt-ended siRNA” is defined as an siRNA having no terminal overhangs. That is, at least one end of the double-stranded compound is blunt. siRNAs whether canonical or blunt act to elicit dsRNAse enzymes and trigger the recruitment or activation of the RNAi antisense mechanism. In a further embodiment, single-stranded RNAi (ssRNAi) compounds that act via the RNAi antisense mechanism are contemplated.

Further modifications can be made to the double-stranded compounds and may include conjugate groups attached to one of the termini, selected nucleobase positions, sugar positions or to one of the internucleoside linkages. Alternatively, the two strands can be linked via a non-nucleic acid moiety or linker group. When formed from only one strand, the compounds can take the form of a self-complementary hairpin-type molecule that doubles back on itself to form a duplex. Thus, the compounds can be fully or partially double-stranded. When formed from two strands, or a single strand that takes the form of a self-complementary hairpin-type molecule doubled back on itself to form a duplex, the two strands (or duplex-forming regions of a single strand) are complementary when they base pair in Watson-Crick fashion.

The oligomeric compounds provided herein may comprise a complementary oligomeric compound from about 8 to about 80 nucleobases (i.e. from about 8 to about 80 linked nucleosides). In other words, a single-stranded compound comprises from 8 to about 80 nucleobases, and a double-stranded antisense compound (such as a siRNA, for example) comprises two strands, each of which is from about 8 to about 80 nucleobases. For double stranded antisense compounds, each strand is independently 8 to 80 nucleobases in length. As used herein, double stranded compounds wherein each strand is “independently 8 to 80 nucleobases in length” refers to compounds in which the strands can be of the same or different length, but each strand is between 8 and 80 nucleobases. Contained within the oligomeric compounds provided herein (whether single or double stranded and on at least one strand) are antisense portions. The “antisense portion” is that part of the oligomeric compound that is designed to work by one of the aforementioned antisense mechanisms. One of ordinary skill in the art will appreciate that this comprehends antisense portions of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41,42, 43, 44, 45,46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds have antisense portions of 10 to 50 nucleobases. One having ordinary skill in the art will appreciate that this embodies antisense compounds having antisense portions of length between and including 10 to 50 nucleobases as exemplified above.

In one embodiment, the antisense compounds have antisense portions of 13 to 80 nucleobases. One having ordinary skill in the art will appreciate that this embodies antisense compounds having antisense portions of length between and including 13 to 80 nucleobases as exemplified above.

In one embodiment, the antisense compounds have antisense portions of 13 to 50 nucleobases. One having ordinary skill in the art will appreciate that this embodies antisense compounds having antisense portions of length between and including 13 to 50 nucleobases as exemplified above.

In one embodiment, the antisense compounds have antisense portions of 13 to 30 nucleobases. One having ordinary skill in the art will appreciate that this embodies antisense compounds having antisense portions of length between and including 13 to 30 nucleobases as exemplified above.

In some embodiments, the antisense compounds have antisense portions of 13 to 24 nucleobases. One having ordinary skill in the art will appreciate that this embodies antisense compounds having antisense portions of 13, 14, 15, 16, 17, 18, 19, 20, 21, 22, 23 or 24 nucleobases.

In one embodiment, the antisense compounds have antisense portions of 19 to 23 nucleobases. One having ordinary skill in the art will appreciate that this embodies antisense compounds having antisense portions of 19, 20, 21, 22 or 23 nucleobases.

In one embodiment, the antisense compounds have antisense portions of 20 to 80 nucleobases. One having ordinary skill in the art will appreciate that this embodies antisense compounds having antisense portions of length between and including 20 to 80 nucleobases as exemplified above.

In one embodiment, the antisense compounds have antisense portions of 20 to 50 nucleobases. One having ordinary skill in the art will appreciate that this embodies antisense compounds having antisense portions of length between and including 20 to 50 nucleobases as exemplified above.

In one embodiment, the antisense compounds have antisense portions of 20 to 30 nucleobases. One having ordinary skill in the art will appreciate that this embodies antisense compounds having antisense portions of 20, 21, 22, 23, 24, 25, 26, 27, 28, 29, or 30 nucleobases.

In one embodiment, the antisense compounds have antisense portions of 20 to 24 nucleobases. One having ordinary skill in the art will appreciate that this embodies antisense compounds having antisense portions of 20, 21, 22, 23, or 24 nucleobases.

In one embodiment, the antisense compounds have antisense portions of 18 to 22 nucleobases. One having ordinary skill in the art will appreciate that this embodies antisense compounds having antisense portions of 18, 19, 20, 21, or 22 nucleobases.

Antisense compounds 8-80 nucleobases in length comprising a stretch of at least eight (8) consecutive nucleobases selected from within the illustrative antisense compounds are considered to be suitable antisense compounds as well.

Compounds described herein include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Other compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that compounds may be represented by oligonucleotide sequences that comprise at least 8 consecutive nucleobases from an internal portion of the sequence of an illustrative compound, and may extend in either or both directions until the oligonucleotide contains about 8 about 80 nucleobases.

Compounds need not be 100% identical to those taught in the instant disclosure. It is understood by those skilled in the art that a compound may include some mismatch nucleobases and maintain function to modulate the expression of IL4-R alpha. In an embodiment, compounds are at least about 70% identical to those taught, more preferably at least about 75% identical, even more preferably at least about 80% identical. Progressively more preferably, compounds are at least about 85%, 90%, 91%, 92%, 93%, 94%, 95%, 96%, 97%, 98%, 99% identical to the compounds provided herein.

One having skill in the art armed with the antisense compounds illustrated herein will be able, without undue experimentation, to identify further antisense compounds.

Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. In formiing oligonucleotides, the phosphate groups covalently link adjacent nucleosides to one another to form a linear polymeric compound. In turn, the respective ends of this linear polymeric compound can be further joined to form a circular compound. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages

Specific examples of oligomeric compounds include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Oligomeric compounds can have one or more modified internucleoside linkages. Modified oligonucleotide backbones containing a phosphorus atom therein include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, phosphonoacetate and thiophosphonoacetate (see Sheehan et al., Nucleic Acids Research, 2003, 31(14), 4109-4118 and Dellinger et al., J. Am. Chem. Soc., 2003, 125, 940-950), selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage, i.e., a single inverted nucleoside residue which may be abasic (the nucleobase is missing or has a hydroxyl group in place thereof). Various salts, mixed salts and free acid forms are also included.

N3′-P5′-phosphoramidates have been reported to exhibit both a high affinity towards a complementary RNA strand and nuclease resistance (Gryaznov et al., J. Am. Chem. Soc., 1994, 116, 3143-3144). N3′-P5′-phosphoramidates have been studied with some success in vivo to specifically down regulate the expression of the c-myc gene (Skorski et al., Proc. Natl. Acad. Sci., 1997, 94, 3966-3971; and Faira et al., Nat. Biotechnol., 2001, 19, 40-44).

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In some embodiments, oligomeric compounds may have one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Amide internucleoside linkages are disclosed in the above referenced U.S. Pat. No. 5,602,240.

Some oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Modified Sugars

Oligomeric compounds may also contain one or more substituted sugar moieties. Suitable compounds can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—(CH₂)₂—O—(CH₂)₂—N(CH₃)₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and, 6,147,200.

DNA-Like and RNA-Like Conformations

The terms used to describe the conformational geometry of homoduplex nucleic acids are “A Form” for RNA and “B Form” for DNA. In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger et al., Principles of Nucleic Acid Structure, 1984, Springer-Verlag; New York, N.Y.). As used herein, B-form geometry is inclusive of both C2′-endo pucker and O4′-endo pucker.

The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). Consequently, compounds that favor an A-form geometry can enhance stacking interactions, thereby increasing the relative Tm and potentially enhancing a compound's antisense effect.

In one aspect, oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA-like nucleosides so that particular properties of an oligomeric compound can be enhanced while maintaining the desirable 3′-endo conformational geometry.

There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include but are not limited to: modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. Also provided herein are oligomeric triggers of RNAi having one or more nucleosides modified in such a way as to favor a C3′-endo type conformation.

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Representative 2′-substituent groups amenable to the present disclosure that give A-form conformational properties (3′-endo) to the resultant duplexes include 2′-O-alkyl, 2′-O-substituted alkyl and 2′-fluoro substituent groups. Other suitable substituent groups are various alkyl and aryl ethers and thioethers, amines and monoalkyl and dialkyl substituted amines.

Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA™, Morita et al, Bioorganic & Medicinal Chemistry Letters (2002), 12, 73-76.)

It is further intended that multiple modifications can be made to one or more of the oligomeric compounds at multiple sites of one or more monomeric subunits (nucleosides are suitable) and or internucleoside linkages to enhance properties such as but not limited to activity in a selected application.

The synthesis of numerous of the modified nucleosides amenable to the present disclosure are known in the art (see for example, Chemistry of Nucleosides and Nucleotides Vol 1-3, ed. Leroy B. Townsend, 1988, Plenum press). The conformation of modified nucleosides and their oligomers can be estimated by various methods routine to those skilled in the art such as molecular dynamics calculations, nuclear magnetic resonance spectroscopy and CD measurements.

Oligonucleotide Mimetics

Another group of oligomeric compounds includes oligonucleotide mimetics. The term “mimetic” as it is applied to oligonucleotides includes oligomeric compounds wherein the furanose ring or the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid.

One such oligomeric compound, an oligonucleotide mimetic that has been shown to have excellent hybridization properties, is referred to as a peptide nucleic acid (PNA) (Nielsen et al., Science, 1991, 254, 1497-1500). PNAs have favorable hybridization properties, high biological stability and are electrostatically neutral molecules. PNA compounds have been used to correct aberrant splicing in a transgenic mouse model (Sazani et al., Nat. Biotechnol., 2002, 20, 1228-1233). In PNA oligomeric compounds, the sugar-backbone of an oligonucleotide is replaced with an amide containing backbone, in particular an aminoethylglycine backbone. The nucleobases are bound directly or indirectly to aza nitrogen atoms of the amide portion of the backbone. Representative United States patents that teach the preparation of PNA oligomeric compounds include, but are not limited to, U.S. Pat. No. 5,539,082; 5,714,331; and 5,719,262. PNA compounds can be obtained commercially from Applied Biosystems (Foster City, Calif., USA). Numerous modifications to the basic PNA backbone are known in the art; particularly useful are PNA compounds with one or more amino acids conjugated to one or both termini. For example, 1-8 lysine or arginine residues are useful when conjugated to the end of a PNA molecule.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups have been selected to give a non-ionic oligomeric compound. Morpholino-based oligomeric compounds are non-ionic mimetics of oligonucleotides which are less likely to form undesired interactions with cellular proteins (Dwaine A. Braasch and David R. Corey, Biochemistry, 2002, 41(14), 4503-4510). Morpholino-based oligomeric compounds have been studied in zebrafish embryos (see: Genesis, volume 30, issue 3, 2001 and Heasman, J., Dev. Biol., 2002, 243, 209-214). Further studies of morpholino-based oligomeric compounds have also been reported (Nasevicius et al., Nat. Genet., 2000, 26, 216-220; and Lacerra et al., Proc. Natl. Acad. Sci., 2000, 97, 9591-9596). Morpholino-based oligomeric compounds are disclosed in U.S. Pat. No. 5,034,506. The morpholino class of oligomeric compounds have been prepared having a variety of different linking groups joining the monomeric subunits. Linking groups can be varied from chiral to achiral, and from charged to neutral. U.S. Pat. No. 5,166,315 discloses linkages including —O—P(═O)(N(CH₃)₂)—O—; U.S. Pat. No. 5,034,506 discloses achiral intermorpholino linkages; and U.S. Pat. No. 5,185,444 discloses phosphorus containing chiral intermorpholino linkages.

A further class of oligonucleotide mimetic is referred to as cyclohexene nucleic acids (CeNA). In CeNA oligonucleotides, the furanose ring normally present in a DNA or RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. coli RNase H resulting in cleavage of the target RNA strand.

A further modification includes bicyclic sugar moieties such as “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et-al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. LNA's are commercially available from ProLigo (Paris, France and Boulder, Colo., USA).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research, 2003, 21, 6365-6372).

Another similar bicyclic sugar moiety that has been prepared and studied has the bridge going from the 3′-hydroxyl group via a single methylene group to the 4′ carbon atom of the sugar ring thereby forming a 3′-C,4′-C-oxymethylene linkage (see U.S. Pat. No. 6,043,060).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11° C.) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands. DNA:LNA chimeras have been shown to efficiently inhibit gene expression when targeted to a variety of regions (5′-untranslated region, region of the start codon or coding region) within the luciferase mRNA (Braasch et al., Nucleic Acids Research, 2002, 30, 5160-5167).

Potent and nontoxic antisense oligonucleotides containing LNAs have been described (Wahlestedt et al., Proc. Natl. Acad. Sci. U.S.A., 2000, 97, 5633-5638). The authors have demonstrated that LNAs confer several desired properties. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished. Further successful in vivo studies involving LNA's have shown knock-down of the rat delta opioid receptor without toxicity (Wahlestedt et al., Proc. Natl. Acad. Sci., 2000, 97, 5633-5638) and in another study showed a blockage of the translation of the large subunit of RNA polymerase II (Fluiter et al., Nucleic Acids Res., 2003, 31, 953-962).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Another oligonucleotide mimetic that has been prepared and studied is threose nucleic acid. This oligonucleotide mimetic is based on threose nucleosides instead of ribose nucleosides. Initial interest in (3′,2′)-alpha-L-threose nucleic acid (TNA) was directed to the question of whether a DNA polymerase existed that would copy the TNA. It was found that certain DNA polymerases are able to copy limited stretches of a TNA template (reported in Chemical and Engineering News, 2003, 81, 9). In another study it was determined that TNA is capable of antiparallel Watson-Crick base pairing with complementary DNA, RNA and TNA oligonucleotides (Chaput et al., J. Am. Chem. Soc., 2003, 125, 856-857).

In one study (3′,2′)-alpha-L-threose nucleic acid was prepared and compared to the 2′ and 3′ amidate analogs (Wu et al., Organic Letters, 2002, 4(8), 1279-1282). The amidate analogs were shown to bind to RNA and DNA with comparable strength to that of RNA/DNA.

Further oligonucleotide mimetics have been prepared to include bicyclic and tricyclic nucleoside analogs (see Steffens et al., Helv. Chim. Acta, 1997, 80, 2426-2439; Steffens et al., J. Am. Chem. Soc., 1999, 121, 3249-3255; Renneberg et al., J. Am. Chem. Soc., 2002, 124, 5993-6002; and Renneberg et al., Nucleic acids res., 2002, 30, 2751-2757). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids which incorporate a phosphorus group in the backbone. This class of oligonucleotide mimetic is reported to have useful physical and biological and pharmacological properties in the areas of inhibiting gene expression (antisense oligonucleotides, sense oligonucleotides and triplex-forming oligonucleotides), as probes for the detection of nucleic acids and as auxiliaries for use in molecular biology. Further oligonucleotide mimetics amenable to the present disclosure have been prepared wherein a cyclobutyl ring replaces the naturally occurring furanosyl ring.

Modified and Alternate Nucleobases

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C). Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in the art as suitable for increasing the binding affinity of the compounds provided herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692.

Oligomeric compounds can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K.-Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K.-Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Grant Publications 20030207804 and 20030175906).

Further helix-stabilizing properties have been observed when a cytosine analog/substitute has an aminoethoxy moiety attached to the rigid 1,3-diazaphenoxazine-2-one scaffold (Lin, K.-Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° C. relative to 5-methyl cytosine (dC5^{me}), which is a high affinity enhancement for a single modification. On the other hand, the gain in helical stability does not compromise the specificity of the oligonucleotides.

Further tricyclic heterocyclic compounds and methods of use are disclosed in U.S. Pat. Nos. 6,028,183, and 6,007,992.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNase H, enhance cellular uptake and exhibit an increased antisense activity (Lin, K-Y; Matteucci, M. J. Am. Chem. Soc. 1998, 120, 8531-8532). The activity enhancement was even more pronounced in case of G-clamp, as a single substitution was shown to significantly improve the in vitro potency of a 20 mer 2′-deoxyphosphorothioate oligonucleotides (Flanagan, W. M.; et al., Proc. Natl. Acad. Sci. USA, 1999, 96, 3513-3518).

Further modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Pre-Grant Publication 20030158403.

Conjugates

Another modification of the oligomeric compounds involves chemically linking to the oligomeric compound one or more moieties or conjugates which enhance the properties of the oligomeric compound, such as to enhance the activity, cellular distribution or cellular uptake of the oligomeric compound. These moieties or conjugates can include conjugate groups covalently bound to functional groups such as primary or secondary hydroxyl groups. Representative conjugate groups are disclosed in PCT Publication WO 93/07883 and U.S. Pat. Nos. 6,287,860 and 6,762,169.

Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligomeric compounds may also be conjugated to drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. Pat. No. 6,656,730.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds can also be modified to have one or more stabilizing groups that are generally attached to one or both termini of an oligomeric compound to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270). These terminal modifications protect the oligomeric compounds having terminal nucleic acid molecules from exonuclease degradation, and can improve delivery and/or localization within a cell. The cap can be present at either the 5′-terminus (5′-cap) or at the 3′-terminus (3′-cap) or can be present on both termini of a single strand, or one or more termini of both strands of a double-stranded compound. This cap structure is not to be confused with the inverted methylguanosine “5′ cap” present at the 5′ end of native mRNA molecules. In non-limiting examples, the 5′-cap includes inverted abasic residue (moiety), 4′,5′-methylene nucleotide; 1-(beta-D-erythrofuranosyl)nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270). For siRNA constructs, the 5′ end (5′ cap) is commonly but not limited to 5′-hydroxyl or 5′-phosphate.

Particularly suitable 3′-cap structures include, for example 4′,5′-methylene nucleotide; 1-(beta-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Further 3′ and 5′-stabilizing groups that can be used to cap one or both ends of an oligomeric compound to impart nuclease stability include those disclosed in WO 03/004602 published on Jan. 16, 2003.

Chimeric Compounds

It is not necessary for all positions in a given oligomeric compound to be uniformly modified, and in fact more than one of the aforementioned modifications may be incorporated in a single compound or even within a single nucleoside within an oligomeric compound.

The present disclosure also includes oligomeric compounds which are chimeric compounds. “Chimeric” oligomeric compounds or “chimeras,” in the context of the present disclosure, are single-or double-stranded oligomeric compounds, such as oligonucleotides, which contain two or more chemically distinct regions, each comprising at least one monomer unit, i.e., a nucleotide in the case of an oligonucleotide compound. Chimeric antisense oligonucleotides are one form of oligomeric compound. These oligonucleotides typically contain at least one region which is modified so as to confer upon the oligonucleotide increased resistance to nuclease degradation, increased cellular uptake, alteration of charge, increased stability and/or increased binding affinity for the target nucleic acid. An additional region of the oligonucleotide may serve as a substrate for RNAses or other enzymes. By way of example, RNAse H is a cellular endonuclease which cleaves the RNA strand of an RNA:DNA duplex. Activation of RNase H, therefore, results in cleavage of the RNA target when bound by a DNA-like oligomeric compound, thereby greatly enhancing the efficiency of oligonucleotide-mediated inhibition of gene expression. The cleavage of RNA:RNA hybrids can, in like fashion, be accomplished through the actions of endoribonucleases, such as RNase III or RNAseL which cleaves both cellular and viral RNA. Cleavage products of the RNA target can be routinely detected by gel electrophoresis and, if necessary, associated nucleic acid hybridization techniques known in the art.

Chimeric oligomeric compounds can be formed as composite structures of two or more oligonucleotides, modified oligonucleotides, oligonucleosides, oligonucleotide mimetics, or regions or portions thereof. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

A “gapmer” is defined as an oligomeric compound, generally an oligonucleotide, having a 2′-deoxyoligonucleotide region flanked by non-deoxyoligonucleotide segments. The central region is referred to as the “gap.” The flanking segments are referred to as “wings.” While not wishing to be bound by theory, the gap of the gapmer presents a substrate recognizable by RNase H when bound to the RNA target whereas the wings do not provide such a substrate but can confer other properties such as contributing to duplex stability or advantageous pharmacokinetic effects. Each wing can be one or more non-deoxyoligonucleotide monomers (if one of the wings has zero non-deoxyoligonucleotide monomers, a “hemimer” is described). In one embodiment, the gapmer is a ten deoxynucleotide gap flanked by five non-deoxynucleotide wings. This is referred to as a 5-10-5 gapmer. Other configurations are readily recognized by those skilled in the art. In one embodiment the wings comprise 2′-MOE modified nucleotides. In another embodiment the gapmer has a phosphorothioate backbone. In another embodiment the gapmer has 2′-MOE wings and a phosphorothioate backbone. Other suitable modifications are readily recognizable by those skilled in the art.

Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds can be conveniently and routinely made through the well-known technique of solid phase synthesis using methods and equipment well known to those skilled in the art. Precursor compounds, including amidites and their intermediates can be purchased or prepared by methods routine to those skilled in the art. The preparation of such precursor compounds for oligonucleotide synthesis are routine in the art and disclosed in U.S. Pat. No. 6,426,220 and published PCT WO 02/36743. Non-commercially available oligonucleosides can by synthesized by methods well known to those skilled in the art.

Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates.

Hybridization

“Hybridization” means the pairing of complementary strands of oligomeric compounds. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refers to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

Complementarity

“Complementarity,” as used herein, refers to the capacity for precise pairing between two nucleobases on one or two oligomeric compound strands. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The antisense compounds provided herein are at least 70%, at least 75%, at least 80%, at least 85%, at least 90%, at least 95%, at least 96%, at least 97%, at least 98% or at least 99% complementary to a target nucleic acid sequence. Percent complementarity of an antisense compound with a target nucleic acid can be determined routinely using programs and methods well known in the art.

The oligomeric compounds also include variants in which a different base is present at one or more of the nucleotide positions in the compound. For example, if the first nucleotide is an adenosine, variants may be produced which contain thymidine, guanosine or cytidine at this position. This may be done at any of the positions of the oligomeric compound. These compounds are then tested using the methods described herein to determine their ability to inhibit expression of IL-4R alpha mRNA.

Identity

Oligomeric compounds, or a portion thereof, may have a defined percent identity to a SEQ ID NO, or a compound having a specific Isis number. This identity may be over the entire length of the oligomeric compound, or in a portion of the oligomeric compound (e.g., nucleobases 1-20 of a 27-mer may be compared to a 20-mer to determine percent identity of the oligomeric compound to the SEQ ID NO.) It is understood by those skilled in the art that an oligonucleotide need not have an identical sequence to those described herein to function similarly to the oligonucleotides described herein. Shortened (i.e., deleted, and therefore non-identical) versions of oligonucleotides taught herein, or non-identical (i.e., one base replaced with another) versions of the oligonucleotides taught herein fall within the scope of the present disclosure. Percent identity is calculated according to the number of bases that are identical to the SEQ ID NO or compound to which it is being compared. The non-identical bases may be adjacent to each other, dispersed through out the oligonucleotide, or both.

For example, a 16-mer having the same sequence as nucleobases 2-17 of a 20-mer is 80% identical to the 20-mer. Alternatively, a 20-mer containing four nucleobases not identical to the 20-mer is also 80% identical to the 20-mer. A 14-mer having the same sequence as nucleobases 1-14 of an 18-mer is 78% identical to the 18-mer. Such calculations are well within the ability of those skilled in the art.

The percent identity is based on the percent of nucleobases in the original sequence present in a portion of the modified sequence. Therefore, a 30 nucleobase oligonucleotide comprising the full sequence of a 20 nucleobase SEQ ID NO would have a portion of 100% identity with the 20 nucleobase SEQ ID NO while further comprising an additional 10 nucleobase portion. As provided herein, the full length of the modified sequence may constitute a single portion.

Target Nucleic Acids

“Targeting” an oligomeric compound to a particular target nucleic acid molecule can be a multistep process. The process usually begins with the identification of a target nucleic acid whose expression is to be modulated. As used herein, the terms “target nucleic acid” and “nucleic acid encoding IL-4R alpha” encompass DNA encoding IL-4R alpha, RNA (including pre-mRNA and mRNA) transcribed from such DNA, and also cDNA derived from such RNA. For example, the target nucleic acid can be a cellular gene (or mRNA transcribed from the gene) whose expression is associated with a particular disorder or disease state, or a nucleic acid molecule from an infectious agent. As disclosed herein, the target nucleic acid encodes IL-4R alpha.

Target Regions, Segments, and Sites

The targeting process usually also includes determination of at least one target region, segment, or site within the target nucleic acid for the antisense interaction to occur such that the desired effect, e.g., modulation of expression, will result. “Region” is defined as a portion of the target nucleic acid having at least one identifiable structure, function, or characteristic. Regions include, but are not limited to start codon region, stop codon region, splice junction region, intron-exon junction region, 5′-cap region, 5′-untranslated region, 3′-untranslated region, translation initiation region, open reading frame, and coding region. Identification of such regions is well within the ability of those skilled in the art. Regions defined by a small number of bases (e.g. start and stop codon, splice junctions) include the region around the small number of bases wherein the region includes at least about a 20, preferably at least about a 30, more preferably at least about a 40, most preferably at least about a 50 nucleobase region including the small number of bases. Within regions of target nucleic acids are segments. “Segments” are defined as smaller or sub-portions of regions within a target nucleic acid. “Sites,” as used herein, are defined as unique nucleobase positions within a target nucleic acid.

Variants

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Consequently, the types of variants described herein are also suitable target nucleic acids.

Target Names, Synonyms, Features

Provided herein are compositions and methods for modulating the expression of IL-4R alpha (also known as Interleukin 4 alpha receptor; CD124; IL-4Ra; interleukin 4 receptor alpha chain). Table 1 lists the GenBank accession numbers of sequences corresponding to nucleic acid molecules encoding IL-4R alpha (nt=nucleotide). Table 1 also describes features contained within the gene target nucleic acid sequences. Representative features include 5′UTR, start codon, coding sequence (CDS), stop codon, 3′UTR, exon, intron, exon:exon junction, intron:exon junction and exon:intron junction. “Feature start site” and “feature end site” refer to the first (5′-most) and last (3′-most) nucleotide numbers, respectively, of the described feature with respect to the designated sequence. For example, for a sequence containing a start codon comprising the first three nucleotides, “feature start site” is “1” and “feature end site” is “3”. The Genbank Accession numbers and the sequences to which they refer are hereby incorporated by reference.

TABLE 1
Gene Targets and Features

| | | | Feature | Feature | SEQ |
|---|---|---|---|---|---|
| | | | Start | End | ID |
| Species | Genbank # | Feature | Site | Site | NO |
| Human | BM738518.1 | exon | 107 | 130 | 128 |
| Human | BM738518.1 | intron:exon junction | 130 | 131 | 128 |
| Human | BM738518.1 | exon | 342 | 429 | 128 |
| Human | BM738518.1 | start codon | 360 | 362 | 128 |
| Human | BM738518.1 | exon:exon junction | 429 | 430 | 128 |
| Human | nt 18636000 to 18689000 of NT_010393.14 | exon | 1472 | 1495 | 129 |
| Human | nt 18636000 to 18689000 of NT_010393.14 | intron:exon junction | 1495 | 1496 | 129 |
| Human | nt 18636000 to 18689000 of NT_010393.14 | intron | 1496 | 17540 | 129 |
| Human | nt 18636000 to 18689000 of NT_010393.14 | intron:exon junction | 17540 | 17541 | 129 |
| Human | nt 18636000 to 18689000 of NT_010393.14 | exon | 17541 | 17673 | 129 |
| Human | nt 18636000 to 18689000 of NT_010393.14 | intron:exon junction | 17673 | 17674 | 129 |
| Human | nt 18636000 to 18689000 of NT_010393.14 | intron | 17674 | 27660 | 129 |
| Human | nt 18636000 to 18689000 of NT_010393.14 | intron:exon junction | 27660 | 27661 | 129 |
| Human | nt 18636000 to 18689000 of NT_010393.14 | exon | 27661 | 27748 | 129 |
| Human | nt 18636000 to 18689000 of NT_010393.14 | start codon | 27679 | 27681 | 129 |
| Human | nt 18636000 to 18689000 of NT_010393.14 | intron:exon junction | 27748 | 27749 | 129 |
| Human | nt 18636000 to 18689000 of NT_010393.14 | intron | 27749 | 29595 | 129 |
| Human | nt 18636000 to 18689000 of NT_010393.14 | intron:exon junction | 29595 | 29596 | 129 |
| Human | nt 18636000 to 18689000 of NT_010393.14 | exon | 29596 | 29734 | 129 |
| Human | nt 18636000 to 18689000 of NT_010393.14 | intron:exon junction | 29734 | 29735 | 129 |
| Human | nt 18636000 to 18689000 of NT_010393.14 | intron | 29735 | 32343 | 129 |
| Human | nt 18636000 to 18689000 of NT_010393.14 | intron:exon junction | 32343 | 32344 | 129 |
| Human | nt 18636000 to 18689000 of NT_010393.14 | exon | 32344 | 32495 | 129 |
| Human | nt 18636000 to 18689000 of NT_010393.14 | intron:exon junction | 32495 | 32496 | 129 |
| Human | nt 18636000 to 18689000 of NT_010393.14 | intron | 32496 | 33941 | 129 |
| Human | nt 18636000 to 18689000 of NT_010393.14 | intron:exon junction | 33941 | 33942 | 129 |
| Human | nt 18636000 to 18689000 of NT_010393.14 | exon | 33942 | 34093 | 129 |
| Human | nt 18636000 to 18689000 of NT_010393.14 | intron:exon junction | 34093 | 34094 | 129 |
| Human | nt 18636000 to 18689000 of NT_010393.14 | intron | 34094 | 40014 | 129 |
| Human | nt 18636000 to 18689000 of NT_010393.14 | intron:exon junction | 40014 | 40015 | 129 |
| Human | nt 18636000 to 18689000 of NT_010393.14 | exon | 40015 | 40171 | 129 |
| Human | nt 18636000 to 18689000 of NT_010393.14 | intron:exon junction | 40171 | 40172 | 129 |
| Human | nt 18636000 to 18689000 of NT_010393.14 | intron | 40172 | 43282 | 129 |
| Human | nt 18636000 to 18689000 of NT_010393.14 | intron:exon junction | 43282 | 43283 | 129 |
| Human | nt 18636000 to 18689000 of NT_010393.14 | exon | 43283 | 43382 | 129 |
| Human | nt 18636000 to 18689000 of NT_010393.14 | intron:exon junction | 43382 | 43383 | 129 |
| Human | nt 18636000 to 18689000 of NT_010393.14 | intron | 43383 | 46390 | 129 |
| Human | nt 18636000 to 18689000 of NT_010393.14 | intron:exon junction | 46390 | 46391 | 129 |
| Human | nt 18636000 to 18689000 of NT_010393.14 | exon | 46391 | 46469 | 129 |
| Human | nt 18636000 to 18689000 of NT_010393.14 | intron:exon junction | 46469 | 46470 | 129 |
| Human | nt 18636000 to 18689000 of NT_010393.14 | intron | 46470 | 48240 | 129 |
| Human | nt 18636000 to 18689000 of NT_010393.14 | intron:exon junction | 48240 | 48241 | 129 |
| Human | nt 18636000 to 18689000 of NT_010393.14 | exon | 48241 | 48290 | 129 |
| Human | nt 18636000 to 18689000 of NT_010393.14 | intron:exon junction | 48290 | 48291 | 129 |
| Human | nt 18636000 to 18689000 of NT_010393.14 | intron | 48291 | 49726 | 129 |
| Human | nt 18636000 to 18689000 of NT_010393.14 | intron:exon junction | 49726 | 49727 | 129 |
| Human | nt 18636000 to 18689000 of NT_010393.14 | exon | 49727 | 52249 | 129 |
| Human | nt 18636000 to 18689000 of NT_010393.14 | stop codon | 51303 | 51305 | 129 |
| Human | nt 18636000 to 18689000 of NT_010393.14 | 3′UTR | 51306 | 52249 | 129 |
| Human | X52425.1 | exon | 1 | 24 | 2 |
| Human | X52425.1 | 5′UTR | 1 | 175 | 2 |
| Human | X52425.1 | exon:exon junction | 24 | 25 | 2 |
| Human | X52425.1 | exon | 25 | 157 | 2 |
| Human | X52425.1 | exon:exon junction | 157 | 158 | 2 |
| Human | X52425.1 | exon | 158 | 245 | 2 |
| Human | X52425.1 | start codon | 176 | 178 | 2 |
| Human | X52425.1 | CDS | 176 | 2653 | 2 |
| Human | X52425.1 | exon:exon junction | 245 | 246 | 2 |
| Human | X52425.1 | exon | 246 | 384 | 2 |
| Human | X52425.1 | exon:exon junction | 384 | 385 | 2 |
| Human | X52425.1 | exon | 385 | 536 | 2 |
| Human | X52425.1 | exon:exon junction | 536 | 537 | 2 |
| Human | X52425.1 | exon | 537 | 688 | 2 |
| Human | X52425.1 | exon:exon junction | 688 | 689 | 2 |
| Human | X52425.1 | exon | 689 | 845 | 2 |
| Human | X52425.1 | exon:exon junction | 845 | 846 | 2 |
| Human | X52425.1 | exon | 846 | 945 | 2 |
| Human | X52425.1 | exon:exon junction | 945 | 946 | 2 |
| Human | X52425.1 | exon | 946 | 1024 | 2 |
| Human | X52425.1 | exon:exon junction | 1024 | 1025 | 2 |
| Human | X52425.1 | exon | 1025 | 1074 | 2 |
| Human | X52425.1 | exon:exon junction | 1074 | 1075 | 2 |
| Human | X52425.1 | exon | 1075 | 3597 | 2 |
| Human | X52425.1 | stop codon | 2651 | 2653 | 2 |
| Human | X52425.1 | 3′UTR | 2654 | 3597 | 2 |
| Mouse | AF000304.1 | exon | 1 | 88 | 130 |
| Mouse | AF000304.1 | start codon | 19 | 21 | 130 |
| Mouse | AF000304.1 | CDS | 19 | 2451 | 130 |
| Mouse | AF000304.1 | exon:exon junction | 88 | 89 | 130 |
| Mouse | AF000304.1 | exon | 89 | 230 | 130 |
| Mouse | AF000304.1 | exon:exon junction | 230 | 231 | 130 |
| Mouse | AF000304.1 | exon | 231 | 382 | 130 |
| Mouse | AF000304.1 | exon:exon junction | 382 | 383 | 130 |
| Mouse | AF000304.1 | exon | 383 | 534 | 130 |
| Mouse | AF000304.1 | exon:exon junction | 534 | 535 | 130 |
| Mouse | AF000304.1 | exon | 535 | 691 | 130 |
| Mouse | AF000304.1 | exon:exon junction | 691 | 692 | 130 |
| Mouse | AF000304.1 | exon | 692 | 791 | 130 |
| Mouse | AF000304.1 | exon:exon junction | 791 | 792 | 130 |
| Mouse | AF000304.1 | exon | 792 | 870 | 130 |
| Mouse | AF000304.1 | exon:exon junction | 870 | 871 | 130 |
| Mouse | AF000304.1 | exon | 871 | 920 | 130 |
| Mouse | AF000304.1 | exon:exon junction | 920 | 921 | 130 |
| Mouse | assembled from M64868.1 and M64879.1 | exon | 996 | 1055 | 131 |
| Mouse | assembled from M64868.1 and M64879.1 | intron:exon junction | 1055 | 1056 | 131 |
| Mouse | assembled from M64868.1 and M64879.1 | intron | 1056 | 1080 | 131 |
| Mouse | assembled from M64868.1 and M64879.1 | exon | 1206 | 1381 | 131 |
| Mouse | assembled from M64868.1 and M64879.1 | intron:exon junction | 1381 | 1382 | 131 |
| Mouse | assembled from M64868.1 and M64879.1 | intron | 1382 | 1406 | 131 |
| Mouse | assembled from M64868.1 and M64879.1 | exon | 1532 | 1619 | 131 |
| Mouse | assembled from M64868.1 and M64879.1 | start codon | 1550 | 1552 | 131 |
| Mouse | assembled from M64868.1 and M64879.1 | intron:exon junction | 1619 | 1620 | 131 |
| Mouse | assembled from M64868.1 and M64879.1 | intron | 1620 | 1644 | 131 |
| Mouse | assembled from M64868.1 and M64879.1 | exon | 1770 | 1911 | 131 |
| Mouse | assembled from M64868.1 and M64879.1 | intron:exon junction | 1911 | 1912 | 131 |
| Mouse | assembled from M64868.1 and M64879.1 | intron | 1912 | 1936 | 131 |
| Mouse | assembled from M64868.1 and M64879.1 | exon | 2062 | 2213 | 131 |
| Mouse | assembled from M64868.1 and M64879.1 | intron:exon junction | 2213 | 2214 | 131 |
| Mouse | assembled from M64868.1 and M64879.1 | intron | 2214 | 2238 | 131 |
| Mouse | assembled from M64868.1 and M64879.1 | exon | 2364 | 2515 | 131 |
| Mouse | assembled from M64868.1 and M64879.1 | intron:exon junction | 2515 | 2516 | 131 |
| Mouse | assembled from M64868.1 and M64879.1 | intron | 2516 | 2540 | 131 |
| Mouse | assembled from M64868.1 and M64879.1 | exon | 2666 | 2822 | 131 |
| Mouse | assembled from M64868.1 and M64879.1 | intron:exon junction | 2822 | 2823 | 131 |
| Mouse | assembled from M64868.1 and M64879.1 | intron | 2823 | 2847 | 131 |
| Mouse | assembled from M64868.1 and M64879.1 | exon | 2973 | 3086 | 131 |
| Mouse | assembled from M64868.1 and M64879.1 | stop codon | 2990 | 2992 | 131 |
| Mouse | assembled from M64868.1 and M64879.1 | intron:exon junction | 3086 | 3087 | 131 |
| Mouse | assembled from M64868.1 and M64879.1 | intron | 3087 | 3111 | 131 |
| Mouse | assembled from M64868.1 and M64879.1 | exon | 3237 | 3336 | 131 |
| Mouse | assembled from M64868.1 and M64879.1 | intron:exon junction | 3336 | 3337 | 131 |
| Mouse | assembled from M64868.1 and M64879.1 | intron | 3337 | 3361 | 131 |
| Mouse | assembled from M64868.1 and M64879.1 | exon | 3487 | 3565 | 131 |
| Mouse | assembled from M64868.1 and M64879.1 | intron:exon junction | 3565 | 3566 | 131 |
| Mouse | assembled from M64868.1 and M64879.1 | intron | 3566 | 3590 | 131 |
| Mouse | assembled from M64868.1 and M64879.1 | exon | 3716 | 3765 | 131 |
| Mouse | assembled from M64868.1 and M64879.1 | intron:exon junction | 3765 | 3766 | 131 |
| Mouse | assembled from M64868.1 and M64879.1 | intron | 3766 | 3790 | 131 |
| Mouse | assembled from M64868.1 and M64879.1 | exon | 3916 | 6358 | 131 |
| Mouse | assembled from M64868.1 and M64879.1 | CDS | 4643 | 5446 | 131 |
| Mouse | assembled from M64868.1 and M64879.1 | 3′UTR | 5447 | 6058 | 131 |
| Mouse | BB867141.1 | exon:exon junction | 58 | 59 | 132 |
| Mouse | BB867141.1 | exon | 59 | 146 | 132 |
| Mouse | BB867141.1 | start codon | 77 | 79 | 132 |
| Mouse | BB867141.1 | exon:exon junction | 146 | 147 | 132 |
| Mouse | BB867141.1 | exon | 147 | 288 | 132 |
| Mouse | BB867141.1 | exon:exon junction | 288 | 289 | 132 |
| Mouse | BB867141.1 | exon | 289 | 440 | 132 |
| Mouse | BB867141.1 | exon:exon junction | 440 | 441 | 132 |
| Mouse | BC012309.1 | CDS | 313 | 1116 | 133 |
| Mouse | BC012309.1 | 3′UTR | 1117 | 1728 | 133 |
| Mouse | M27959.1 | 5′UTR | 1 | 236 | 134 |
| Mouse | M27959.1 | exon:exon junction | 42 | 43 | 134 |
| Mouse | M27959.1 | exon | 43 | 218 | 134 |
| Mouse | M27959.1 | exon:exon junction | 218 | 219 | 134 |
| Mouse | M27959.1 | exon | 219 | 306 | 134 |
| Mouse | M27959.1 | start codon | 237 | 239 | 134 |
| Mouse | M27959.1 | CDS | 237 | 2669 | 134 |
| Mouse | M27959.1 | exon:exon junction | 306 | 307 | 134 |
| Mouse | M27959.1 | exon | 307 | 448 | 134 |
| Mouse | M27959.1 | exon:exon junction | 448 | 449 | 134 |
| Mouse | M27959.1 | exon | 449 | 600 | 134 |
| Mouse | M27959.1 | exon:exon junction | 600 | 601 | 134 |
| Mouse | M27959.1 | exon | 601 | 752 | 134 |
| Mouse | M27959.1 | exon:exon junction | 752 | 753 | 134 |
| Mouse | M27959.1 | exon | 753 | 909 | 134 |
| Mouse | M27959.1 | 3′UTR | 816 | 3583 | 134 |
| Mouse | M27959.1 | exon:exon junction | 909 | 910 | 134 |
| Mouse | M27959.1 | exon | 910 | 1009 | 134 |
| Mouse | M27959.1 | exon:exon junction | 1009 | 1010 | 134 |
| Mouse | M27959.1 | exon | 1010 | 1088 | 134 |
| Mouse | M27959.1 | exon:exon junction | 1088 | 1089 | 134 |
| Mouse | M27959.1 | exon | 1089 | 1138 | 134 |
| Mouse | M27959.1 | exon:exon junction | 1138 | 1139 | 134 |
| Mouse | M27959.1 | 3′UTR | 2670 | 3281 | 134 |
| Mouse | M27960.1 | 5′UTR | 1 | 236 | 135 |
| Mouse | M27960.1 | exon:exon junction | 42 | 43 | 135 |
| Mouse | M27960.1 | exon | 43 | 218 | 135 |
| Mouse | M27960.1 | exon:exon junction | 218 | 219 | 135 |
| Mouse | M27960.1 | exon | 219 | 306 | 135 |
| Mouse | M27960.1 | start codon | 237 | 239 | 135 |
| Mouse | M27960.1 | CDS | 237 | 929 | 135 |
| Mouse | M27960.1 | exon:exon junction | 306 | 307 | 135 |
| Mouse | M27960.1 | exon | 307 | 448 | 135 |
| Mouse | M27960.1 | exon:exon junction | 448 | 449 | 135 |
| Mouse | M27960.1 | exon | 449 | 600 | 135 |
| Mouse | M27960.1 | exon:exon junction | 600 | 601 | 135 |
| Mouse | M27960.1 | exon | 601 | 752 | 135 |
| Mouse | M27960.1 | exon:exon junction | 752 | 753 | 135 |
| Mouse | M27960.1 | exon | 753 | 909 | 135 |
| Mouse | M27960.1 | exon:exon junction | 909 | 910 | 135 |
| Mouse | M27960.1 | exon | 910 | 1023 | 135 |
| Mouse | M27960.1 | stop codon | 927 | 929 | 135 |
| Mouse | M27960.1 | 3′UTR | 930 | 3697 | 135 |
| Mouse | M27960.1 | exon:exon junction | 1023 | 1024 | 135 |
| Mouse | M27960.1 | exon | 1024 | 1123 | 135 |
| Mouse | M27960.1 | exon:exon junction | 1123 | 1124 | 135 |
| Mouse | M27960.1 | exon | 1124 | 1202 | 135 |
| Mouse | M27960.1 | exon:exon junction | 1202 | 1203 | 135 |
| Mouse | M27960.1 | exon | 1203 | 1252 | 135 |
| Mouse | M27960.1 | exon:exon junction | 1252 | 1253 | 135 |
| Mouse | M27960.1 | CDS | 1980 | 2783 | 135 |
| Mouse | M27960.1 | 3'UTR | 2784 | 3395 | 135 |
| Mouse | M29854.1 | exon:exon junction | 26 | 27 | 1 |
| Mouse | M29854.1 | exon | 27 | 202 | 1 |
| Mouse | M29854.1 | exon:exon junction | 202 | 203 | 1 |
| Mouse | M29854.1 | exon | 203 | 290 | 1 |
| Mouse | M29854.1 | start codon | 221 | 223 | 1 |
| Mouse | M29854.1 | CDS | 221 | 2653 | 1 |
| Mouse | M29854.1 | exon:exon junction | 290 | 291 | 1 |
| Mouse | M29854.1 | exon | 291 | 432 | 1 |
| Mouse | M29854.1 | exon:exon junction | 432 | 433 | 1 |
| Mouse | M29854.1 | exon | 433 | 584 | 1 |
| Mouse | M29854.1 | exon:exon junction | 584 | 585 | 1 |
| Mouse | M29854.1 | exon | 585 | 736 | 1 |
| Mouse | M29854.1 | exon:exon junction | 736 | 737 | 1 |
| Mouse | M29854.1 | exon | 737 | 893 | 1 |
| Mouse | M29854.1 | exon:exon junction | 893 | 894 | 1 |
| Mouse | M29854.1 | exon | 894 | 993 | 1 |
| Mouse | M29854.1 | exon:exon junction | 993 | 994 | 1 |
| Mouse | M29854.1 | exon | 994 | 1072 | 1 |
| Mouse | M29854.1 | exon:exon junction | 1072 | 1073 | 1 |
| Mouse | M29854.1 | exon | 1073 | 1122 | 1 |
| Mouse | M29854.1 | exon:exon junction | 1122 | 1123 | 1 |
| Mouse | M29854.1 | exon | 1123 | 3565 | 1 |
| Mouse | M29854.1 | 3′UTR | 2654 | 3265 | 1 |
| Mouse | NM_010557.1 | 5′UTR | 1 | 236 | 136 |
| Mouse | NM_010557.1 | exon:exon junction | 42 | 43 | 136 |
| Mouse | NM_010557.1 | exon | 43 | 218 | 136 |
| Mouse | NM_010557.1 | exon:exon junction | 218 | 219 | 136 |
| Mouse | NM_010557.1 | exon | 219 | 306 | 136 |
| Mouse | NM_010557.1 | start codon | 237 | 239 | 136 |
| Mouse | NM_010557.1 | CDS | 237 | 929 | 136 |
| Mouse | NM_010557.1 | exon:exon junction | 306 | 307 | 136 |
| Mouse | NM_010557.1 | exon | 307 | 448 | 136 |
| Mouse | NM_010557.1 | exon:exon junction | 448 | 449 | 136 |
| Mouse | NM_010557.1 | exon | 449 | 600 | 136 |
| Mouse | NM_010557.1 | exon:exon junction | 600 | 601 | 136 |
| Mouse | NM_010557.1 | exon | 601 | 752 | 136 |
| Mouse | NM_010557.1 | exon:exon junction | 752 | 753 | 136 |
| Mouse | NM_010557.1 | exon | 753 | 909 | 136 |
| Mouse | NM_010557.1 | exon:exon junction | 909 | 910 | 136 |
| Mouse | NM_010557.1 | exon | 910 | 1023 | 136 |
| Mouse | NM_010557.1 | stop codon | 927 | 929 | 136 |
| Mouse | NM_010557.1 | 3′UTR | 930 | 3697 | 136 |
| Mouse | NM_010557.1 | exon:exon junction | 1023 | 1024 | 136 |
| Mouse | NM_010557.1 | exon | 1024 | 1123 | 136 |
| Mouse | NM_010557.1 | exon:exon junction | 1123 | 1124 | 136 |
| Mouse | NM_010557.1 | exon | 1124 | 1202 | 136 |
| Mouse | NM_010557.1 | exon:exon junction | 1202 | 1203 | 136 |
| Mouse | NM_010557.1 | exon | 1203 | 1252 | 136 |
| Mouse | NM_010557.1 | exon:exon junction | 1252 | 1253 | 136 |
| Mouse | NM_010557.1 | CDS | 1980 | 2783 | 136 |
| Mouse | NM_010557.1 | 3′UTR | 2784 | 3395 | 136 |

Modulation of Target Expression

Modulation of expression of a target nucleic acid can be achieved through alteration of any number of nucleic acid (DNA or RNA) functions. “Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of IL-4R alpha. Thus, in one embodiment modulation of expression can mean increase or decrease in target RNA or protein levels. In another embodiment modulation of expression can mean an increase or decrease of one or more RNA splice products, or a change in the ratio of two or more splice products.

The effect of oligomeric compounds on target nucleic acid expression can be tested in any of a variety of cell types provided that the target nucleic acid is present at measurable levels. The use of primary cell lines is also contemplated. The effect of oligomeric compounds on target nucleic acid expression can be routinely determined using, for example, PCR or Northern blot analysis. Such methods and cell lines are well known to those skilled in the art.

Assaying Modulation of Expression

Modulation of IL-4R alpha expression can be assayed in a variety of ways known in the art. IL-4R alpha mRNA levels can be quantitated by, e.g., Northern blot analysis, competitive polymerase chain reaction (PCR), or real-time PCR. RNA analysis can be performed on total cellular RNA or poly(A)+mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Validated Target Segments

The locations on the target nucleic acid to which active oligomeric compounds hybridize are hereinbelow referred to as “validated target segments.” As used herein the term “validated target segment” is defined as at least an 8-nucleobase portion of a target region to which an active oligomeric compound is targeted. While not wishing to be bound by theory, it is presently believed that these target segments represent portions of the target nucleic acid which are accessible for hybridization.

Target segments can include DNA or RNA sequences that comprise at least the 8 consecutive nucleobases from the 5′-terminus of a validated target segment (the remaining nucleobases being a consecutive stretch of the same DNA or RNA beginning immediately upstream of the 5′-terminus of the target segment and continuing until the DNA or RNA contains about 8 to about 80 nucleobases). Similarly validated target segments are represented by DNA or RNA sequences that comprise at least the 8 consecutive nucleobases from the 3′-terminus of a validated target segment (the remaining nucleobases being a consecutive stretch of the same DNA or RNA beginning immediately downstream of the 3′-terminus of the target segment and continuing until the DNA or RNA contains about 8 to about 80 nucleobases). It is also understood that a validated oligomeric target segment can be represented by DNA or RNA sequences that comprise at least 8 consecutive nucleobases from an internal portion of the sequence of a validated target segment, and can extend in either or both directions until the oligonucleotide contains about 8 about 80 nucleobases.

Screening for Modulator Compounds

In another embodiment, the validated target segments identified herein can be employed in a screen for additional compounds that modulate the expression of IL-4R alpha. “Modulators” are those compounds that modulate the expression of IL-4R alpha and which comprise at least an 8-nucleobase portion which is complementary to a validated target segment. The screening method comprises the steps of contacting a validated target segment of a nucleic acid molecule encoding IL-4R alpha with one or more candidate modulators, and selecting for one or more candidate modulators which perturb the expression of a nucleic acid molecule encoding IL-4R alpha. Once it is shown that the candidate modulator or modulators are capable of modulating the expression of a nucleic acid molecule encoding IL-4R alpha, the modulator can then be employed in further investigative studies of the function of IL-4R alpha, or for use as a research, diagnostic, or therapeutic agent. The validated target segments can also be combined with a second strand as disclosed herein to form stabilized double-stranded (duplexed) oligonucleotides for use as a research, diagnostic, or therapeutic agent.

Phenotypic Assays

Once modulator compounds of IL-4R alpha have been identified by the methods disclosed herein, the compounds can be further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of IL-4R alpha in health and disease.

Kits, Research Reagents, Diagnostics, and Therapeutics

The oligomeric compounds can be utilized for diagnostics, therapeutics, prophylaxis and as research reagents and kits. Furthermore, antisense compounds, which are able to inhibit gene expression with specificity, are often used by those of ordinary skill to elucidate the function of particular genes or to distinguish between functions of various members of a biological pathway.

For use in kits and diagnostics, the oligomeric compounds, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more compounds or compositions provided herein are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns. Examples of methods of gene expression analysis known in the art include DNA arrays or microarrays.

Compounds described herein can be used to modulate the expression of IL-4R alpha in an animal, such as a human. In one non-limiting embodiment, the methods comprise the step of administering to said animal an effective amount of an antisense compound that inhibits expression of IL-4R alpha. In one embodiment, the antisense compounds effectively inhibit the levels or function of IL-4R alpha RNA. Because reduction in IL-4R alpha mRNA levels can lead to alteration in IL-4R alpha protein products of expression as well, such resultant alterations can also be measured. Antisense compounds that effectively inhibit the levels or function of IL-4R alpha RNA or protein products of expression are considered an active antisense compound. In one embodiment, the antisense compounds inhibit the expression of IL-4R alpha causing a reduction of RNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of IL-4R alpha can be measured in a bodily fluid, tissue or organ of the animal. Bodily fluids include, but are not limited to, blood (serum or plasma), lymphatic fluid, cerebrospinal fluid, semen, urine, synovial fluid and saliva and can be obtained by methods routine to those skilled in the art. Tissues or organs include, but are not limited to, blood (e.g., hematopoietic cells, such as human hematopoietic progenitor cells, human hematopoietic stem cells, CD34+ cells CD4+ cells), lymphocytes and other blood lineage cells, skin, bone marrow, spleen, thymus, lymph node, brain, spinal cord, heart, skeletal muscle, liver, pancreas, prostate, kidney, lung, oral mucosa, esophagus, stomach, ilium, small intestine, colon, bladder, cervix, ovary, testis, mammary gland, adrenal gland, and adipose (white and brown). Samples of tissues or organs can be routinely obtained by biopsy. In some alternative situations, samples of tissues or organs can be recovered from an animal after death.

The cells contained within said fluids, tissues or organs being analyzed can contain a nucleic acid molecule encoding IL-4R alpha protein and/or the IL-4R alpha-encoded protein itself. For example, fluids, tissues or organs procured from an animal can be evaluated for expression levels of the target mRNA or protein. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the target gene expression in the aforementioned fluids, tissues or organs, collected from an animal contacted with one or more compounds, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

The compounds described herein can be utilized in pharmaceutical compositions by adding an effective amount of a compound to a suitable pharmaceutically acceptable diluent or carrier. In one aspect, the compounds inhibit the expression of IL-4R alpha. The compounds can also be used in the manufacture of a medicament for the treatment of diseases and disorders related to IL-4R alpha expression.

Methods whereby bodily fluids, organs or tissues are contacted with an effective amount of one or more of the antisense compounds or compositions provided herein are also contemplated. Bodily fluids, organs or tissues can be contacted with one or more of the compounds resulting in modulation of IL-4R alpha expression in the cells of bodily fluids, organs or tissues. An effective amount can be determined by monitoring the modulatory effect of the antisense compound or compounds or compositions on target nucleic acids or their products by methods routine to the skilled artisan. Further contemplated are ex vivo methods of treatment whereby cells or tissues are isolated from a subject, contacted with an effective amount of the antisense compound or compounds or compositions and reintroduced into the subject by routine methods known to those skilled in the art.

In one embodiment, provided are uses of a compound of an isolated double stranded RNA oligonucleotide in the manufacture of a medicament for inhibiting IL-4R alpha expression or overexpression. Thus, provided herein is the use of an isolated double stranded RNA oligonucleotide targeted to IL-4R alpha in the manufacture of a medicament for the treatment of a disease or disorder by means of the methods described above.

Salts, Prodrugs and Bioequivalents

The oligomeric compounds described herein comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the oligomeric compounds described herein, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides are prepared as SATE ((S-acetyl-2-thioethyl)phosphate) derivatives according to the methods disclosed in WO 93/24510 or WO 94/26764.

The term “pharmaceutically acceptable salts” refers to physiologically and pharmaceutically acceptable salts of the compounds provided herein: i.e., salts that retain the desired biological activity of the parent compound and do not impart undesired toxicological effects thereto.

Formulations

Compositions and methods for the formulation of oligonucleotides are well known to those skilled in the art. One of skill in the art will recognize that formulations are routinely designed according to their intended use, i.e. route of administration. For example, oral or topical formulations may include at least one penetration enhancer to enhance the delivery of a compound whereas the same compound may be delivery intravenously without the need for penetration enhancers.

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

Combinations

Compositions described herein can contain two or more oligomeric compounds. In another related embodiment, compositions can contain one or more antisense compounds, particularly oligonucleotides, targeted to a first nucleic acid and one or more additional antisense compounds targeted to a second nucleic acid target. Alternatively, compositions can contain two or more antisense compounds targeted to different regions of the same nucleic acid target. Two or more combined compounds may be used together or sequentially.

Nonlimiting Disclosure and Incorporation by Reference

While certain compounds, compositions and methods provided herein have been described with specificity in accordance with certain embodiments, the following examples serve only to illustrate the compounds provided herein and are not intended to limit the same. Each of the references, GenBank accession numbers, and the like recited in the present application is incorporated herein by reference in its entirety.

### EXAMPLES

### Example 1

### Cell Types

The effect of oligomeric compounds on target nucleic acid expression was tested in the following cell types.

A549:

The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

b.END:

The mouse brain endothelial cell line b.END was obtained from Dr. Werner Risau at the Max Plank Institute (Bad Nauheim, Germany). b.END cells were routinely cultured in DMEM, high glucose (Invitrogen Life Technologies; Carlsbad, Calif.) supplemented with 10% fetal bovine serum (Invitrogen Life Technologies, Carlsbad, Calif.). Cells were routinely passaged by trypsinization and dilution when they reached approximately 90% confluence. Cells were seeded into 96-well plates (Falcon-Primaria #353872, BD Biosciences, Bedford, Mass.) at a density of approximately 3000 cells/well for use in oligomeric compound transfection experiments.

Treatment with Oligomeric Compounds

When cells reached appropriate confluency, they were treated with oligonucleotide using a transfection method as described.

Lipofectin™

When cells reached 65-75% confluency, they were treated with single or double stranded oligonucleotides. Oligonucleotide was mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of oligonucleotide and a LIPOFECTIN™ concentration of 2.5 or 3 μg/mL per 100 nM oligonucleotide. This transfection mixture was incubated at room temperature for approximately 0.5 hours. For cells grown in 96-well plates, wells were washed once with 100 μL OPTI-MEM™-1 and then treated with 130 μL of the transfection mixture. Cells grown in 24-well plates or other standard tissue culture plates are treated similarly, using appropriate volumes of medium and oligonucleotide. Cells are treated and data are obtained in duplicate or triplicate. After approximately 4-7 hours of treatment at 37° C., the medium containing the transfection mixture was replaced with fresh culture medium. Cells were harvested 16-24 hours after oligonucleotide treatment.

Other Transfection Reagents

A number of commercially available transfection reagents are available that can be used with the methods disclosed in the application. These reagents include, but are not limited to Cytofectin™ (Gene Therapy Systems, San Diego, Calif.), Lipofectamine™ (Invitrogen Life Technologies, Carlsbad, Calif.), Oligofectamine™ (Invitrogen Life Technologies, Carlsbad, Calif.), and FuGENE™ (Roche Diagnostics Corp., Indianapolis, Ind.) using methods provided in the manufacture's instructions. Oligonucleotides can also be delivered to cells by electroporation using methods well known to those skilled in the art.

### Example 2

### Real-Time Quantitative PCR Analysis of IL-4R Alpha mRNA Levels

Quantitation of IL-4R alpha mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA was subjected to sequential reverse transcriptase (RT) reaction and real-time PCR, both of which were performed in the same well. RT and PCR reagents were obtained from Invitrogen Life Technologies (Carlsbad, Calif.). RT, real-time PCR was carried out in the same by adding 20 μL PCR cocktail (2.5× PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5× ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycle of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

Presented in Table 2 are primers and probes used to measure GAPDH expression in the cell types described herein. The GAPDH PCR probes have JOE covalently linked to the 5′ end and TAMRA or MGB covalently linked to the 3′ end, where JOE is the fluorescent reporter dye and TAMRA or MGB is the quencher dye. In some cell types, primers and probe designed to a GAPDH sequence from a different species are used to measure GAPDH expression. For example, a human GAPDH primer and probe set is used to measure GAPDH expression in monkey-derived cells and cell lines.

TABLE 2
GAPDH primers and probes for use in real-time PCR

| | | | | SEQ |
|---|---|---|---|---|
| Target | | Sequence | | ID |
| Name | Species | Description | Sequence (5′ to 3′) | NO |
| GAPDH | Human | Forward Primer | CAACGGATTTGGTCGTATTGG | 137 |
| GAPDH | Human | Reverse Primer | GGCAACAATATCCACTTTACCAGAGT | 138 |
| GAPDH | Human | Probe | CGCCTGGTCACCAGGGCTGCT | 139 |
| GAPDH | Human | Forward Primer | GAAGGTGAAGGTCGGAGTC | 140 |
| GAPDH | Human | Reverse Primer | GAAGATGGTGATGGGATTTC | 141 |
| GAPDH | Human | Probe | CAAGCTTCCCGTTCTCAGCC | 142 |
| GAPDH | Human | Probe | TGGAATCATATTGGAAGATG | 143 |
| GAPDH | Mouse | Forward Primer | GGCAAATTCAACGGCACAGT | 144 |
| GAPDH | Mouse | Reverse Primer | GGGTCTCGCTCCTGGAAGAT | 145 |
| GAPDH | Mouse | Probe | AAGGCCGAGAATGGGAAGCTTGTCATC | 146 |
| GAPDH | Rat | Forward Primer | TGTTCTAGAGACAGCCGCATCTT | 147 |
| GAPDH | Rat | Reverse Primer | CACCGACCTTCACCATCTTGT | 148 |
| GAPDH | Rat | Probe | TTGTGCAGTGCCAGCCTCGTCTCA | 149 |

Probes and primers for use in real-time PCR were designed to hybridize to target-specific sequences. The primers and probes and the target nucleic acid sequences to which they hybridize are presented in Table 3. The target-specific PCR probes have FAM covalently linked to the 5′ end and TAMRA or MGB covalently linked to the 3′ end, where FAM is the fluorescent dye and TAMRA or MGB is the quencher dye.

TABLE 3

Gene target-specific primers and probes
for use in real-time PCR

| | | | | SEQ |
|---|---|---|---|---|
| Target | | Sequence | | ID |
| Name | Species | Description | Sequence (5′ to 3′) | NO |
| IL-4R alpha | Human | Fwd Primer | AATGGTCCCACCAATTGCA | 150 |
| IL-4R alpha | Human | Reverse Primer | CTCCGTTGTTCTCAGGGATACAC | 151 |
| IL-4R alpha | Human | Probe | TTTTTCTGCTCTCCGAAGCCC | 152 |
| IL-4R alpha | Mouse | Fwd Primer | TCCCATTTTGTCCACCGAATA | 153 |
| IL-4R alpha | Mouse | Reverse Primer | GTTTCTAGGCCCAGCTTCCA | 154 |
| IL-4R alpha | Mouse | Probe | TGTCACTCAAGGCTCTCAGCGGTCC | 155 |

### Example 3

### Design and Screening of Duplexed Oligomeric Compounds Targeting IL-4R Alpha

A series of duplexes, including dsRNA and mimetics thereof, comprising oligomeric compounds provided herein and their complements were designed to target IL-4R alpha. The nucleobase sequence of the antisense strand of the duplex comprised at least a portion of an oligonucleotide targeted to IL-4R alpha as disclosed herein. The ends of the strands may be modified by the addition of one or more natural or modified nucleobases to form an overhang. The sense strand of the nucleic acid duplex is then designed and synthesized as the complement of the antisense strand and may also contain modifications or additions to either terminus. The antisense and sense strands of the duplex comprise from about 17 to 25 (i.e., 17, 18, 19, 20, 21, 22, 23, 24, or 25) nucleotides, or from about 19 to 23 nucleotides. Alternatively, the antisense and sense strands comprise 20, 21 or 22 nucleotides.

For example, in one embodiment, both strands of the dsRNA duplex were complementary over the central nucleobases, each having overhangs at one or both termini.

For example, a duplex comprising an antisense strand having the sequence CGAGAGGCGGACGGGACCG (SEQ ID NO: 156) and having a two-nucleobase overhang of deoxythymidine(dT) would have the following structure:

| | | |
|---|---|---|
| cgagaggcggacgggaccgTT | Antisense Strand | (SEQ ID NO: 157) |
| \|\|\|\|\|\|\|\|\|\|\|\|\|\|\|\|\|\|\| | | |
| TTgcucuccgccugcccuggc | Complement | (SEQ ID NO: 158) |

Overhangs can range from 2 to 6 nucleobases and these nucleobases may or may not be complementary to the target nucleic acid. In another embodiment, the duplexes have an overhang on only one terminus.

In another embodiment, a duplex comprising an antisense strand having the same sequence, for example CGAGAGGCGGACGGGACCG (SEQ ID NO: 156), can be prepared with blunt ends (no single stranded overhang) as shown:

| | | |
|---|---|---|
| cgagaggcggacgggaccg | Antisense Strand | (SEQ ID NO: 156) |
| \|\|\|\|\|\|\|\|\|\|\|\|\|\|\|\|\|\|\| | | |
| gcucuccgccugcccuggc | Complement | (SEQ ID NO: 159) |

The RNA duplex can be unimolecular or bimolecular; i.e., the two strands can be part of a single molecule or may be separate molecules.

RNA strands of the duplex can be synthesized by methods routine to the skilled artisan or purchased from Dharmacon Research Inc. (Lafayette, Colo.). Once synthesized, the complementary strands are annealed. The single strands are aliquotted and diluted to a concentration of 50 μM. Once diluted, 30 μL of each strand is combined with 15 μL of a 5× solution of annealing buffer. The final concentration of said buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM.

Once prepared, the duplexed compounds were evaluated for their ability to modulate IL-4R alpha. When cells reached 80% confluency, they were treated with duplexed compounds. Cells were grown in 96-well plates. Wells were washed once with 200 μL OPTI-MEM-1™ reduced-serum medium (Gibco BRL) and then treated with 130 μL of OPTI-MEM-1™ containing 12 μg/mL LIPOFECTIN™ (Gibco BRL) and the desired duplex antisense compound at a final concentration of 200 nM (a ratio of 6 μg/mL LIPOFECTIN™ per 100 nM duplex antisense compound). After 5 hours of treatment, the medium was replaced with fresh medium. Cells were harvested 16 hours after treatment, at which time RNA is isolated and target reduction measured by RT-PCR.

### Example 4

### Inhibition of Mouse IL-4R Alpha by dsRNA Compounds Having dT Overhangs

A series of double stranded oligomeric compounds was designed to target different portions of mouse IL-4R alpha (GenBank Accession No. M29854.1, SEQ ID NO: 1). The double stranded compounds were RNA with a two base, 3′ dT overhang. All linkages were phosphodiester linkages. The compounds were tested as described above in Examples 2 and 3 for the inhibition of expression of IL-4R alpha in b.END cells at the concentrations indicated. The results are expressed at percent inhibition relative to untreated control. The target regions to which the oligomeric compounds are inhibitory are referred to as “validated target segments.”

TABLE 4

Inhibition of expression of mouse
IL-4R alpha by dsRNA compounds

| | | | | |
|---|---|---|---|---|
| | Sense (S)/ | | SEQ | |
| Isis | antisense | Target | ID | % Inhibition |

| | | | | | |
|---|---|---|---|---|---|
| No. | (AS) | Site | Sequence | NO | Concentration (nM) |

| | | |
|---|---|---|
| mouse sequences from M29854.1 | 0.4 nM | 4.0 nM |

| | | | | | | |
|---|---|---|---|---|---|---|
| 383264 | AS | 65-83 | CCGCTGTTCTCAGGTGACATT | 3 | 8 | 51 |
| 383304 | S | | TGTCACGTGAGAACAGCGGTT | 4 | | |
| 383265 | AS | 194-212 | GGATCTGCTCCCAGCTCTCTT | 5 | 11 | 42 |
| 383305 | S | | GAGAGCTGGGAGCAGATCCTT | 6 | | |
| 383266 | AS | 415-433 | ACCTCAGCAACAACAGCACTT | 7 | 35 | 79 |
| 383306 | S | | GTGCTGTTGTTGCTGAGGTTT | 8 | | |
| 383267 | AS | 495-513 | TACAGCGCACCACACTGACTT | 9 | 0 | 33 |
| 383307 | S | | GTCAGTGTGGTGCGCTGTATT | 10 | | |
| 383268 | AS | 640-658 | GTGAAGGATCTGCTCCCAGTT | 11 | 0 | 68 |
| 383308 | S | | CTGGGAGCAGATCCTTCACTT | 12 | | |
| 383269 | AS | 668-686 | AGCAACAACAGCACACTCATT | 13 | 30 | 59 |
| 383309 | S | | TGAGTGTGCTGTTGTTGCTTT | 14 | | |
| 383270 | AS | 706-724 | CACAGACCTCAGCAACAACTT | 15 | 43 | 68 |
| 383310 | S | | GTTGTTGCTGAGGTCTGTGTT | 16 | | |
| 383271 | AS | 723-741 | AAACAGCTCCATACAGCGCTT | 17 | 24 | 39 |
| 383311 | S | | GCGCTGTATGGAGCTGTTTTT | 18 | | |
| 383272 | AS | 729-747 | CCTCCACATTCTGTACTGGTT | 19 | 11 | 22 |
| 383312 | S | | CCAGTACAGAATGTGGAGGTT | 20 | | |
| 383273 | AS | 729-747 | TTCTGGGAAACCTGCCAGCTT | 21 | 28 | 53 |
| 383313 | S | | GCTGGCAGGTTTCCCAGAATT | 22 | | |
| 383274 | AS | 732-750 | CACTAAAACTCCGGTAGGCTT | 23 | 17 | 40 |
| 383314 | S | | GCCTACCGGAGTTTTAGTGTT | 24 | | |
| 383275 | AS | 775-793 | GTTCTCAGGTGACATGCTCTT | 25 | 26 | 58 |
| 383315 | S | | GAGCATGTCACCTGAGAACTT | 26 | | |
| 383276 | AS | 885-903 | AGCTGGAAGTGGTTGTACCTT | 27 | 13 | 44 |
| 383316 | S | | GGTACAACCACTTCCAGCTTT | 28 | | |
| 383277 | AS | 970-988 | GTCCTCTCTGGAGATGTTGTT | 29 | 19 | 44 |
| 383317 | S | | CAACATCTCCAGAGAGGACTT | 30 | | |
| 383278 | AS | 975-993 | CCGGTAGGCAGGATTGTCTTT | 31 | 7 | 27 |
| 383318 | S | | AGACAATCCTGCCTACCGGTT | 32 | | |
| 383279 | AS | 1096-1114 | GGTTGACTCCTGGCTTCGGTT | 33 | 5 | 28 |
| 383319 | S | | CCGAAGCCAGGAGTCAACCTT | 34 | | |
| 383280 | AS | 1101-1119 | TACTTGGTTGACTCCTGGCTT | 35 | 0 | 49 |
| 383320 | S | | GCCAGGAGTCAACCAAGTATT | 36 | | |
| 383281 | AS | 1295-1313 | TTTATTGACATAAAGCTCCTT | 37 | 52 | 78 |
| 383321 | S | | GGAGGTTTATGTCAATAAATT | 38 | | |
| 383282 | AS | 1306-1324 | TCTGATTGGACCGGCCTATTT | 39 | 13 | 39 |
| 383322 | S | | ATAGGCCGGTCCAATCAGATT | 40 | | |
| 383283 | AS | 1331-1349 | TAGACTATGAATTCTGCAGTT | 41 | 56 | 75 |
| 383323 | S | | CTGCAGAATTCATAGTCTATT | 42 | | |
| 383284 | AS | 1381-1399 | TGTACAGTAAGTTGTTCGATT | 43 | 25 | 51 |
| 383324 | S | | TCGAACAACTTACTGTACATT | 44 | | |

Regular font indicates a ribose sugar. With ribose sugars, T is uracil. Bold indicates a deoxyribose sugar. The following pairs of sequences inhibited expression of mouse IL-4R alpha at least 50% at 4.0 nM: SEQ ID NOs: 3_{—}4; 7_{—}8; 11_{—}12; 13_{—}14; 15_{—}16; 12_{—}22; 25_{—}26; 37_{—}38; 41_{—}42; and 43_{—}44. The following pairs of sequences inhibited expression of mouse L-4R alpha at least 40% at 4.0 nM: SEQ ID NOs: 3_{—}4; 5_{—}6; 35_{—}36; 7_{—}8; 11_{—}12; 13_{—}14; 15_{—}16; 12_{—}22; 23_{—}24; 25_{—}26; 27_{—}28; 29_{—}30; 35_{—}36; 37_{—}38; 41_{—}42; and 43_{—}44.

### Example 5

### Inhibition of Mouse IL-4R Alpha by Blunt Ended dsRNA Compounds

A series of double stranded oligomeric compounds was designed to target different portions of mouse IL-4R alpha (GenBank Accession No. M29854.1, SEQ ID NO: 1). The double stranded compounds are RNA with blunt ends. All linkages are phosphodiester linkages. The compounds were tested as described above in Examples 2 and 3 for the inhibition of expression of IL-4R alpha in b.END cells at the concentrations indicated. The results are expression as percent inhibition relative to untreated control. If present, ND indicates not determined. The target regions to which the oligomeric compounds are inhibitory are referred to as “validated target segments.”

TABLE 4

Inhibition of expression of mouse IL-4R alpha
by dsRNA compounds (Expressed as % inhibition
relative to untreated control)

Sense (S)/

| Isis | Antisense | Target | | SEQ | Concentration (nM) | |
|---|---|---|---|---|---|---|
| No | (AS) | Site | Sequence | ID | 0.4 | 4.0 |

| | | | | | | | | |
|---|---|---|---|---|---|---|---|---|
| 359661 | | Control | TTATCGCTTCTCGTTGCTT | 45 | 9 | 17 | | |
| 359662 | | | AAGCAACGAGAAGCGATAA | 46 | 0.148 | 0.444 | 1.33 | 4 |
| 386727 | AS | 81-99 | P-AAAATCAGAAGCCAGGTCC | 47 | 2 | 11 | 36 | 36 |
| 386747 | S | | GGACCTGGCTTCTGATTTT | 48 | | | | |
| 386728 | AS | 210-228 | P-CAAAAGGTGCCTGCACAAG | 49 | 14 | 0 | 20 | 38 |
| 386748 | S | | CTTGTGCAGGCACCTTTTG | 50 | | | | |
| 386729 | AS | 431-449 | P-TTCAGAGAACTCGAAGAAC | 51 | 12 | 30 | 45 | 59 |
| 386749 | S | | GTTCTTCGAGTTCTCTGAA | 52 | | | | |
| 386730 | AS | 656-647 | P-GTTATTCCAGGTCAGCAGC | 53 | 26 | 22 | 43 | 64 |
| 386750 | S | | GCTGCTGACCTGGAATAAC | 54 | | | | |
| 386731 | AS | 739-757 | P-ATGAATTCTGCAGGGTTGT | 55 | 28 | 38 | 48 | 69 |
| 386751 | S | | ACAACCCTGCAGAATTCAT | 56 | | | | |
| 386732 | AS | 745-763 | TAGACTATGAATTCTGCAG | 57 | 56 | 69 | 73 | 81 |
| 386752 | S | | CTGCAGAATTCATAGTGTA | 58 | | | | |
| 386733 | AS | 748-766 | P-TTATAGACTATGAATTCTG | 59 | 38 | 44 | 55 | 71 |
| 386753 | S | | CAGAATTCATAGTCTATAA | 60 | | | | |
| 386734 | AS | 791-809 | P-GATGTTGATCGGGAAGCTC | 61 | 13 | 15 | 26 | 49 |
| 386754 | S | | GAGCTTCCCGATCAACATC | 62 | | | | |
| 386735 | AS | 1100-1118 | P-AATGCTGAAGTAACAGAAC | 63 | 43 | 51 | 61 | 74 |
| 386755 | S | | GTTCTGTTACTTCAGCATT | 64 | | | | |
| 386736 | AS | 1105-1123 | P-TTGGTAATGCTGAAGTAAC | 65 | 23 | 17 | 25 | 36 |
| 386756 | S | | GTTACTTCAGCATTACCAA | 66 | | | | |
| 386737 | AS | 1585-1603 | P-AAGTCGGAAAACAGGTTCT | 67 | 23 | 13 | 21 | ND |
| 386757 | S | | AGAACCTGTTTTCCGACTT | 68 | | | | |
| 386738 | AS | 2282-2300 | P-AGTAAATAAGGGCACGGAG | 69 | 21 | 8 | 8 | 32 |
| 386758 | S | | CTCCGTGCCCTTATTTACT | 70 | | | | |
| 386739 | AS | 2888-2906 | P-ATTCACCATTCTCTGGATT | 71 | 29 | 47 | 62 | 75 |
| 386759 | S | | AATCCAGAGAATGGTGAAT | 72 | | | | |
| 386740 | AS | 3071-3089 | P-AATGGGACTGAGTAGGTAG | 73 | 47 | 58 | 65 | 77 |
| 386760 | S | | CTACCTACTCAGTCCCATT | 74 | | | | |
| 386741 | AS | 3399-3417 | P-TTTATCTTCCACATCCCAG | 75 | 26 | 43 | 38 | 52 |
| 386761 | S | | CTGGGATGTGGAAGATAAA | 76 | | | | |
| 386742 | AS | 3620-3638 | P-AACAGACTAGGTGTTGCAA | 77 | 37 | 27 | 45 | 58 |
| 386762 | S | | TTGCAACACCTAGTCTGTT | 78 | | | | |
| 386743 | AS | 3662-3680 | P-ATTGACATAAAGCTCCATG | 79 | 25 | 33 | 44 | 50 |
| 386763 | S | | CATGGAGCTTTATGTCAAT | 80 | | | | |
| 386744 | AS | 3665-3683 | P-TTTATTGACATAAAGCTCC | 81 | 68 | 70 | 79 | 83 |
| 386764 | S | | GGAGCTTTATGTCAATAAA | 82 | | | | |
| 386745 | AS | 3667-3685 | P-ACTTTATTGACATAAAGCT | 83 | 24 | 24 | 33 | 35 |
| 386765 | S | | AGCTTTATGTCAATAAAGT | 84 | | | | |
| 386746 | AS | | P-ACCTCAGCAACAACAGCAC | 85 | 17 | 12 | 26 | 27 |
| 386766 | S | | GTGCTGTTGTTGCTGAGGT | 86 | | | | |

Regular font indicates a ribose sugar. With ribose sugars, T is uracil. Bold indicates a deoxyribose sugar. Underline indicates 2′-O-methyl ribose. P— is a 5′ phosphate. The following pairs of sequences inhibited expression of mouse IL-4R alpha at least 50% at 4.OnM: SEQ ID NOs: 51_{—}52; 52_{—}54; 55_{—}56; 57_{—}58; 59_{—}60; 63_{—}64; 71_{—}72; 73_{—}74; 75_{—}76; 77_{—}78; and 79_{—}80.

### Example 6

### Dose Response Curves Using dsRNA Compounds Targeted to Mouse IL-4R Alpha

The most active dsRNA compounds were tested at a series of concentrations. The compounds were tested as described above in Examples 2 and 3 for the inhibition of expression of IL-4R alpha in b.END cells at the concentrations indicated. A reduction in expression is expressed as percent inhibition. The data are presented in Table 5.

TABLE 5
Inhibition of expression of mouse IL-4R alpha by dsRNA compounds- Dose
response (expressed as % inhibition relative to untreated control)

| | | Concentration (nM) | | | | | | | |
|---|---|---|---|---|---|---|---|---|---|
| Isis Nos | SEQ ID NOs | 0.004 | 0.012 | 0.037 | 0.111 | 0.333 | 1.000 | 3.000 | 9.000 |
| 359661_359662 | 45_46 | 7 | 0 | 0 | 0 | 0 | 0 | 4 | 8 |
| 383266_383306 | 7_8 | 6 | 0 | 0 | 6 | 42 | 56 | 72 | 75 |
| 383281_383321 | 37_38 | 15 | 12 | 41 | 52 | 65 | 72 | 79 | 82 |
| 383283_383323 | 41_42 | 12 | 25 | 29 | 45 | 65 | 72 | 79 | 78 |
| 386732_386752 | 57_58 | 12 | 7 | 3 | 22 | 42 | 62 | 75 | 85 |
| 386733_386753 | 59_60 | 0 | 0 | 16 | 24 | 24 | 50 | 62 | 62 |
| 386735_386755 | 63_64 | 0 | 6 | 0 | 0 | 9 | 2 | 15 | 3 |
| 386740_386760 | 73_74 | 7 | 11 | 19 | 36 | 45 | 58 | 68 | 76 |
| 386744_386764 | 81_82 | 14 | 10 | 19 | 43 | 54 | 68 | 75 | 81 |
| 386746_386766 | 85_86 | 0 | 0 | 0 | 0 | 13 | 14 | 23 | 33 |

### Example 7

### Inhibition of Human IL-4R Alpha by dsRNA Compounds Having dT Overhangs

A series of double stranded oligomeric compounds was designed to target different portions of human IL-4R alpha (GenBank Accession No. X52425.1, SEQ ID NO: 2). The double stranded compounds were RNA with a two base, 3′ dT overhang. All linkages were phosphodiester linkages. The compounds were tested as described above in Examples 2 and 3 for the inhibition of expression of IL-4R alpha in A549 cells at the concentrations indicated. The results are expression as percent inhibition relative to untreated control. The target regions to which the oligomeric compounds are inhibitory are referred to as “validated target segments.”

TABLE 6

Inhibition of expression of human IL-4R
alpha by dsRNA compounds

| | | | | |
|---|---|---|---|---|
| | Sense (S)/ | | SEQ | |
| Isis | antisense | Target | ID | % Inhibition |

| | | | | | |
|---|---|---|---|---|---|
| No. | (AS) | Site | Sequence | NO | Concentration (nM) |

| | | |
|---|---|---|
| human sequences from X52425.1 | 0.4 nM | 4.0 nM |

| | | | | | | |
|---|---|---|---|---|---|---|
| 383245 | AS | 168-186 | AGCCACCCCATTGGGAGATTT | 87 | 5 | 35 |
| 383285 | S | | ATCTCCCAATGGGGTGGCTTT | 88 | | |
| 383246 | AS | 2744-2762 | AAGTCTTTTGGAAATCTGCTT | 89 | 48 | 75 |
| 383286 | S | | GCAGATTTCCAAAAGACTTTT | 90 | | |
| 383247 | AS | 2764-2782 | CCTTCATACCATGGTTCTTTT | 91 | 2 | 26 |
| 383287 | S | | AAGAACCATGGTATGAAGGTT | 92 | | |
| 383248 | AS | 3169-3187 | GAGGACCTCTAGGCAATGATT | 93 | 17 | 23 |
| 383288 | S | | TCATTGCCTAGAGGTGCTCTT | 94 | | |
| 383249 | AS | 174-192 | GAGCAAAGCCACCCCATTGTT | 95 | 28 | 66 |
| 383289 | S | | CAATGGGGTGGCTTTGCTCTT | 96 | | |
| 383250 | AS | 3054-3072 | CAGTGAGACAGAGGCAGGTTT | 97 | 22 | 58 |
| 383290 | S | | ACCTGCCTCTGTCTCACTGTT | 98 | | |
| 383251 | AS | 497-515 | AGCCCTTCCACAGCAGCTGTT | 99 | 24 | 61 |
| 383291 | S | | CAGCTGCTGTGGAAGGGCTTT | 100 | | |
| 383252 | AS | 2057-2075 | AAGGCTTATACCCCTCTTCTT | 101 | 44 | 70 |
| 383292 | S | | GAAGAGGGGTATAAGCCTTTT | 102 | | |
| 383253 | AS | 2060-2078 | GGAAAGGCTTATACCCCTGTT | 103 | 17 | 61 |
| 383293 | S | | GAGGGGTATAAGCCTTTCCTT | 104 | | |
| 383254 | AS | 507-525 | GGCTTGAAGGAGCCCTTCCTT | 105 | 11 | 23 |
| 383294 | S | | GGAAGGGCTCCTTCAAGCCTT | 106 | | |
| 383255 | AS | 2525-2543 | TACTCTTCTCTGAGATGCCTT | 107 | 56 | 73 |
| 383295 | S | | GGCATCTCAGAGAAGAGTATT | 108 | | |
| 383256 | AS | 2533-2551 | TGAGGATTTACTCTTCTCTTT | 109 | 43 | 72 |
| 383296 | S | | AGAGAAGAGTAAATCCTCATT | 110 | | |
| 383257 | AS | 209-227 | GCAGGACCAGGCAGCTCACTT | 111 | 19 | 25 |
| 383297 | S | | GTGAGCTGCCTGGTCCTGCTT | 112 | | |
| 383258 | AS | 1396-1414 | GTCCAGGAACAGGCTCTCTTT | 113 | 19 | 22 |
| 383298 | S | | AGAGAGCCTGTTCCTGGACTT | 114 | | |
| 383259 | AS | 620-638 | TATACAGGTAATTGTCAGGTT | 115 | 46 | 76 |
| 383299 | S | | CCTGACAATTACCTGTATATT | 116 | | |
| 383260 | AS | 736-754 | AGACTTCAGGGTGCTGGCTTT | 117 | 26 | 45 |
| 383300 | S | | AGCCAGCACCCTGAAGTCTTT | 118 | | |
| 383261 | AS | 17-35 | TCTTTAATTATCTGCGCGCTT | 119 | 7 | 36 |
| 383301 | S | | GCGCGCAGATAATTAAAGATT | 120 | | |
| 383262 | AS | 2793-2811 | TGTTAGGCCAACGTCAGTGTT | 121 | 55 | 73 |
| 383302 | S | | CACTGACGTTGGCCTAACATT | 122 | | |
| 383263 | AS | 3080-3098 | TTAGTTTCTAGGCTCGGCTTT | 123 | 54 | 75 |
| 383303 | S | | AGCCGAGCCTAGAAACTAATT | 124 | | |
| 359661 | | Control | TTATCGCTTCTCGTTGCTT | 45 | 11 | 6 |
| 359662 | | | AAGCAACGAGAAGCGATAA | 46 | | |

Regular font indicates a ribose sugar. With ribose sugars, T is uracil. Bold indicates a deoxyribose sugar. The following pairs of sequences inhibited expression of mouse IL-4R alpha at least 50% at 4.0 nM: SEQ ID NOs: 89_{—}90; 95_{—}96; 97_{—}98; 99_{—}100; 101_{—}102; 103_{—}104; 107_{—}108; 109_{—}110; 115_{—}116; 121_{—}122; 123_{—}124. The following pairs of sequences inhibited expression of mouse IL-4R alpha at least 40% at 4.0 nM: SEQ ID NOs: 89_{—}90; 95_{—}96; 97_{—}98; 99_{—}100; 101_{—}102; 103_{—}104; 107_{—}108; 109_{—}110; 115_{—}116; 117_{—}118; 121_{—}122; 123_{—}124.

### Example 8

### Dose Response Curves Using dsRNA Compounds Targeted to HumanIL-4R Alpha

The most active dsRNA compounds were tested at a series of concentrations. The compounds were tested as described above in Examples 2 and 3 for the inhibition of expression of IL-4R alpha in A549 cells at the concentrations indicated. A reduction in expression is expressed as percent inhibition. The data are presented in Table 7.

TABLE 7
Inhibition of expression of human IL-4R alpha by dsRNA compounds- Dose
response (expressed as % inhibition relative to untreated control)

| | | Concentration (nM) | | | | | | | |
|---|---|---|---|---|---|---|---|---|---|
| Isis No | SEQ ID NOs | 0.006 | 0.017 | 0.049 | 0.148 | 0.444 | 1.333 | 4.000 | 12.000 |
| 359661_359662 | 45_46 | 2 | 0 | 0 | 0 | 0 | 0 | 4 | 8 |
| 383246_383286 | 89_90 | 0 | 4 | 2 | 29 | 42 | 67 | 25 | 87 |
| 383249_383289 | 95_96 | 0 | 0 | 11 | 0 | 11 | 31 | 55 | 66 |
| 383252_383292 | 101_102 | 0 | 3 | 0 | 21 | 37 | 62 | 72 | 86 |
| 383255_383295 | 107_108 | 4 | 3 | 13 | 35 | 59 | 77 | 82 | 78 |
| 383256_383296 | 109_110 | 0 | 0 | 22 | 21 | 33 | 64 | 77 | 88 |
| 383259_383299 | 115_116 | 3 | 1 | 6 | 13 | 43 | 66 | 76 | 84 |
| 383262_383302 | 121_121 | 0 | 0 | 14 | 33 | 58 | 71 | 76 | 79 |
| 383263_383303 | 123_124 | 0 | 1 | 20 | 45 | 60 | 73 | 79 | 75 |

### Example 9

### Mouse Model of Allergic Inflammation

In the mouse model of allergic inflammation, mice were sensitized and challenged with aerosolized chicken ovalbumin (OVA). Airway responsiveness was assessed by inducing airflow obstruction with a methacholine aerosol using a noninvasive method. This method used unrestrained conscious mice that are placed into the main chamber of a plethysmograph (Buxco Electronics, Inc. Troy, N.Y.). Pressure difference between this chamber and a reference chamber were used to extrapolate minute volume, breathing frequency and enhanced pause (Penh). Penh is a dimensionless parameter that is a function of total pulmonary airflow in mice (i.e. the sum of the airflow in the upper and lower respiratory tracts) during the respiratory cycle of the animal; the lower the Penh, the greater the airflow. This parameter closely correlates with lung resistance as measured by traditional, invasive techniques using ventilated animals (Hamelmann et al., Am. J. Respir. Crit. Care Med., 1997, 156:766-775). Dose-response data were plotted as raw Penh values to increasing concentrations of methacholine. This system was used to test the efficacy of antisense oligonucleotide ISIS 231894 (a 5-10-5 MOE gapmer with phosphorothioate linkages at each position and 5-methylcytosines in place of each cytosine residue) as compared to three dsRNAs having the same sequences, but different chemistries. The sequence and chemistry of the compounds are shown below in Table 7. Regular font indicates an unmodified residue. Bold font indicates a 2′-MOE modification, italic font indicates a 2′-OMe modification and underlined font indicates a 2′-F modification.

TABLE 7

IL-4R alpha Compounds Tested in Mouse
Model of Allergic Inflammation

| | | |
|---|---|---|
| | | SEQ |
| | | ID |
| ISIS No. | Sequence | NO: |

| | | |
|---|---|---|
| 231894 | CCGCTGTTCTCAGGTGACAT | 125 |
| 383321_383281 | 5′-GGAGCUUUAUGUCAAUAAAdTdT-3′ | 38 |
| | 3′-dTdTCCUCGAAAUACAGUUAUUU-5′-P | 37 |
| 388219_388220 | 5′-GGAGCUUUAUGUCAAUAAA-3′ | 126 |
| | 3′-CCUCGAAAUACAGUUAUUU-5′-P | 127 |
| 388218_386744 | 5′-GGAGCUUUAUGUCAAUAAA-3′ | 126 |
| | 3′-CCUCGAAAUACAGUUAUUU-5′-P | 127 |

There are several important features common to human asthma and the mouse model of allergic inflammation. One of these is pulmonary inflammation, in which cytokine expression and Th2 profile is dominant. Another is goblet cell hyperplasia with increased mucus production. Lastly, airway hyperresponsiveness (AHR) occurs, resulting in increased sensitivity to cholinergic receptor agonists such as acetylcholine or methacholine. The compositions and methods provided herein may be used to treat AHR and pulmonary inflammation in animals, including humans. The combined use of antisense oligonucleotides to human IL4R-alpha with one or more conventional asthma medications is contemplated.

Ovalbumin Induced Allergic Inflammation Model

Balb/c mice (Charles River Laboratory, Taconic Farms, N.Y.) are maintained in micro-isolator cages housed in a specific pathogen free (SPF) facility. The sentinel cages within the animal colony surveyed negative for viral antibodies and the presence of known mouse pathogens. Mice are sensitized and challenged with aerosol chicken OVA. Briefly, 20 μg of alum precipitated OVA is injected intraperitoneally on days 0 and 14. On days 24, 25 and 26, the animals are exposed for 20 minutes to 1% OVA (in saline) by ultrasonic nebulization. The mice were treated with 100 μg/kg of ISIS 231894 (positive control IL-4Ralpha antisense oligonucleotide) or 200 or 500 μg/kg of one of the three dsRNA compounds on days 59, 61, 63, 66 and 68, delivered by nose only inhalation. A second series of nebulized OVA administrations are given on days 66 and 67 to produce the allergic response. The study endpoints are measured on day 69. Oligonucleotides are suspended in 0.9% sodium chloride and delivered via inhalation using a nose-only exposure system. A Lovelace nebulizer is used to deliver the oligonucleotide, and set at a flow rate of 1.4 liter per minute feeding into a total flow rate of 10 liters per minute. The exposure chamber is equilibrated with an oligonucleotide aerosol solution for 5 minutes before mice were placed in a restraint tubes attached to the chamber. Restrained mice are treated for a total of 10 minutes. Endpoints analyzed include, but are not limited to Penh, inflammatory cell levels in BAL and/or tissue and mucus levels.

A significant reduction in methacholine induced AHR was seen in response to the single stranded ASO 231894 at the 100 μg/kg dose. Each of the dsRNA compounds was effective in significantly (p≦0.05) reducing Penh at one of the two doses as compared to vehicle. The unmodified dsRNA with dT overhangs, 383321_{—}383281, was able to significantly reduce eosinophil recruitment to the lung as was the single stranded compound. No effect was observed on recruitment of other inflammatory cells. Neither the single nor the double stranded compounds reduced the number of periodic acid Schiff base stained goblet cells in the lung, a measure of mucus production. These data demonstrate that both single stranded and double stranded nucleic acid compounds targeted to IL-4R alpha can be effective in the treatment of airway hyperresponsiveness and pulmonary inflammation which can be characteristic of asthma.

What is claimed is:

1. A double stranded antisense compound targeted to a nucleic acid molecule encoding human IL-4R alpha (SEQ ID NO: 2), wherein each strand is independently 13 to 30 nucleobases in length.

2. The compound of claim 1 wherein each strand is independently 19 to 23 nucleobases in length.

3. The compound of claim 1 wherein one strand is at least 80% complementary to said nucleic acid molecule encoding human IL-4R alpha.

4. The compound of claim 1 wherein one strand is at least 90% complementary to said nucleic acid molecule encoding human IL-4R alpha.

5. The compound of claim 1 wherein one strand is at least 95% complementary to said nucleic acid molecule encoding human IL-4R alpha.

6. The compound of claim 1 wherein one strand is 100% complementary to said nucleic acid molecule encoding human IL-4R alpha.

7. The compound of claim 1 wherein one strand is at least 70% identical to SEQ ID NO: 3; 5; 35; 7; 11; 13; 15; 12; 23; 25; 27; 29; 35; 37; 41; 43; 51; 52; 55; 57; 59; 63; 71; 73; 75; 77; 79; 89; 95; 97; 99; 101; 103; 107; 109; 115; 117; 121; or 123.

8. The compound of claim 1 wherein one strand is at least 80% identical to SEQ ID NO: 3; 5; 35; 7; 11; 13; 15; 12; 23; 25; 27; 29; 35; 37; 41; and 43; 51; 52; 55; 57; 59; 63; 71; 73; 75; 77; 79; 89; 95; 97; 99; 101; 103; 107; 109; 115; 117; 121; or 123.

9. The compound of claim 1 wherein one strand is at least 90% identical to SEQ ID NO: 3; 5; 35; 7; 11; 13; 15; 12; 23; 25; 27; 29; 35; 37; 41; 43; 51; 52; 55; 57; 59; 63; 71; 73; 75; 77; 79; 89; 95; 97; 99; 101; 103; 107; 109; 115; 117; 121; or 123.

10. The compound of claim 1 wherein one strand is at least 95% identical to SEQ ID NO: 3; 5; 35; 7; 11; 13; 15; 12; 23; 25; 27; 29; 35; 37; 41; and 43; 51; 52; 55; 57; 59; 63; 71; 73; 75; 77; 79; 89; 95; 97; 99; 101; 103; 107; 109; 115; 117; 121; or 123.

11. The compound of claim 1 wherein one strand is 100% identical to SEQ ID NO: 3; 5; 35; 7; 11; 13; 15; 12; 23; 25; 27; 29; 35; 37; 41; and 43; 51; 52; 55; 57; 59; 63; 71; 73; 75; 77; 79; 89; 95; 97; 99; 101; 103; 107; 109; 115; 117; 121; or 123.

12. The compound of claim 1 comprising dT overhangs.

13. The compound of claim 1 comprising at least one modified internucleoside linkage, sugar moiety, or nucleobase.

14. The compound of claim 13 comprising a chimeric oligonucleotide.

15. A pharmaceutical composition comprising a compound of claim 1 and a pharmaceutically acceptable penetration enhancer, carrier, or diluent.

16. A method of inhibiting the expression of IL-4R alpha in a bodily fluid, cell or tissue comprising contacting said bodily fluid, cell or tissue with the compound of claim 1.

17. A method of reducing airway hyperresponsiveness in an animal comprising administration of the compound of claim 1 to said animal.

18. A method of inhibiting eosinophil recruitment to the lung of an animal comprising administration of the compound of claim 1 to said animal.


SEQUENCE LISTING (159 sequences)

SEQ ID NO: 1 - DNA, 3697 bp, Homo sapiens
cgcaggaaagccccgcgcggcgcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaacccaagtaatgaaaatctgtgactgagtgaccttgggggctgcggtggtgaggagagctcacgggaatcctggagcagtgtagctggcgtgtcaaaagcagaaacgcaggagatggacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggccccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtc

SEQ ID NO: 2 - DNA, 3597 bp, Mus musculus
ggcgaatggagcaggggcgcgcagataattaaagatttacacacagctggaagaaatcatagagaagccgggcgtggtggctcatgcctataatcccagcacttttggaggctgaggcgggcagatcacttgagatcaggagttcgagaccagcctggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctc

SEQ ID NO: 3 - DNA, 21 bp, Artificial Sequence
  Antisense Compound
  misc_feature (1)...(19): Bases at these positions are RNA
ccgcuguucucaggugacatt

SEQ ID NO: 4 - DNA, 21 bp, Artificial Sequence
  Antisense Compound
  misc_feature (1)...(19): Bases at these positions are RNA
ugucaccugagaacagcggtt

SEQ ID NO: 5 - DNA, 21 bp, Artificial Sequence
  Antisense Compound
  misc_feature (1)...(19): Bases at these positions are RNA
ggaucugcucccagcucuctt

SEQ ID NO: 6 - DNA, 21 bp, Artificial Sequence
  Antisense Compound
  misc_feature (1)...(19): Bases at these positions are RNA
gagagcugggagcagaucctt

SEQ ID NO: 7 - DNA, 21 bp, Artificial Sequence
  Antisense Compound
  misc_feature (1)...(19): Bases at these positions are RNA
accucagcaacaacagcactt

SEQ ID NO: 8 - DNA, 21 bp, Artificial Sequence
  Antisense Compound
  misc_feature (1)...(19): Bases at these positions are RNA
gugcuguuguugcugaggutt

SEQ ID NO: 9 - DNA, 21 bp, Artificial Sequence
  Antisense Compound
  misc_feature (1)...(19): Bases at these positions are RNA
uacagcgcaccacacugactt

SEQ ID NO: 10 - DNA, 21 bp, Artificial Sequence
  Antisense Compound
  misc_feature (1)...(19): Bases at these positions are RNA
gucaguguggugcgcuguatt

SEQ ID NO: 11 - DNA, 21 bp, Artificial Sequence
  Antisense Compound
  misc_feature (1)...(19): Bases at these positions are RNA
gugaaggaucugcucccagtt

SEQ ID NO: 12 - DNA, 21 bp, Artificial Sequence
  Antisense Compound
  misc_feature (1)...(19): Bases at these positions are RNA
cugggagcagauccuucactt

SEQ ID NO: 13 - DNA, 21 bp, Artificial Sequence
  Antisense Compound
  misc_feature (1)...(19): Bases at these positions are RNA
agcaacaacagcacacucatt

SEQ ID NO: 14 - DNA, 21 bp, Artificial Sequence
  Antisense Compound
  misc_feature (1)...(19): Bases at these positions are RNA
ugagugugcuguuguugcutt

SEQ ID NO: 15 - DNA, 21 bp, Artificial Sequence
  Antisense Compound
  misc_feature (1)...(19): Bases at these positions are RNA
cacagaccucagcaacaactt

SEQ ID NO: 16 - DNA, 21 bp, Artificial Sequence
  Antisense Compound
  misc_feature (1)...(19): Bases at these positions are RNA
guuguugcugaggucugugtt

SEQ ID NO: 17 - DNA, 21 bp, Artificial Sequence
  Antisense Compound
  misc_feature (1)...(19): Bases at these positions are RNA
aaacagcuccauacagcgctt

SEQ ID NO: 18 - DNA, 21 bp, Artificial Sequence
  Antisense Compound
  misc_feature (1)...(19): Bases at these positions are RNA
gcgcuguauggagcuguuutt

SEQ ID NO: 19 - DNA, 21 bp, Artificial Sequence
  Antisense Compound
  misc_feature (1)...(19): Bases at these positions are RNA
ccuccacauucuguacuggtt

SEQ ID NO: 20 - DNA, 21 bp, Artificial Sequence
  Antisense Compound
  misc_feature (1)...(19): Bases at these positions are RNA
ccaguacagaauguggaggtt

SEQ ID NO: 21 - DNA, 21 bp, Artificial Sequence
  Antisense Compound
  misc_feature (1)...(19): Bases at these positions are RNA
uucugggaaaccugccagctt

SEQ ID NO: 22 - DNA, 21 bp, Artificial Sequence
  Antisense Compound
  misc_feature (1)...(19): Bases at these positions are RNA
gcuggcagguuucccagaatt

SEQ ID NO: 23 - DNA, 21 bp, Artificial Sequence
  Antisense Compound
  misc_feature (1)...(19): Bases at these positions are RNA
cacuaaaacuccgguaggctt

SEQ ID NO: 24 - DNA, 21 bp, Artificial Sequence
  Antisense Compound
  misc_feature (1)...(19): Bases at these positions are RNA
gccuaccggaguuuuagugtt

SEQ ID NO: 25 - DNA, 21 bp, Artificial Sequence
  Antisense Compound
  misc_feature (1)...(19): Bases at these positions are RNA
guucucaggugacaugcuctt

SEQ ID NO: 26 - DNA, 21 bp, Artificial Sequence
  Antisense Compound
  misc_feature (1)...(19): Bases at these positions are RNA
gagcaugucaccugagaactt

SEQ ID NO: 27 - DNA, 21 bp, Artificial Sequence
  Antisense Compound
  misc_feature (1)...(19): Bases at these positions are RNA
agcuggaagugguuguacctt

SEQ ID NO: 28 - DNA, 21 bp, Artificial Sequence
  Antisense Compound
  misc_feature (1)...(19): Bases at these positions are RNA
gguacaaccacuuccagcutt

SEQ ID NO: 29 - DNA, 21 bp, Artificial Sequence
  Antisense Compound
  misc_feature (1)...(19): Bases at these positions are RNA
guccucucuggagauguugtt

SEQ ID NO: 30 - DNA, 21 bp, Artificial Sequence
  Antisense Compound
  misc_feature (1)...(19): Bases at these positions are RNA
caacaucuccagagaggactt

SEQ ID NO: 31 - DNA, 21 bp, Artificial Sequence
  Antisense Compound
  misc_feature (1)...(19): Bases at these positions are RNA
ccgguaggcaggauugucutt

SEQ ID NO: 32 - DNA, 21 bp, Artificial Sequence
  Antisense Compound
  misc_feature (1)...(19): Bases at these positions are RNA
agacaauccugccuaccggtt

SEQ ID NO: 33 - DNA, 21 bp, Artificial Sequence
  Antisense Compound
  misc_feature (1)...(19): Bases at these positions are RNA
gguugacuccuggcuucggtt

SEQ ID NO: 34 - DNA, 21 bp, Artificial Sequence
  Antisense Compound
  misc_feature (1)...(19): Bases at these positions are RNA
ccgaagccaggagucaacctt

SEQ ID NO: 35 - DNA, 21 bp, Artificial Sequence
  Antisense Compound
  misc_feature (1)...(19): Bases at these positions are RNA
uacuugguugacuccuggctt

SEQ ID NO: 36 - DNA, 21 bp, Artificial Sequence
  Antisense Compound
  misc_feature (1)...(19): Bases at these positions are RNA
gccaggagucaaccaaguatt

SEQ ID NO: 37 - DNA, 21 bp, Artificial Sequence
  Antisense Compound
  misc_feature (1)...(19): Bases at these positions are RNA
uuuauugacauaaagcucctt

SEQ ID NO: 38 - DNA, 21 bp, Artificial Sequence
  Antisense Compound
  misc_feature (1)...(19): Bases at these positions are RNA
ggagcuuuaugucaauaaatt

SEQ ID NO: 39 - DNA, 21 bp, Artificial Sequence
  Antisense Compound
  misc_feature (1)...(19): Bases at these positions are RNA
ucugauuggaccggccuautt

SEQ ID NO: 40 - DNA, 21 bp, Artificial Sequence
  Antisense Compound
  misc_feature (1)...(19): Bases at these positions are RNA
auaggccgguccaaucagatt

SEQ ID NO: 41 - DNA, 21 bp, Artificial Sequence
  Antisense Compound
  misc_feature (1)...(19): Bases at these positions are RNA
uagacuaugaauucugcagtt

SEQ ID NO: 42 - DNA, 21 bp, Artificial Sequence
  Antisense Compound
  misc_feature (1)...(19): Bases at these positions are RNA
cugcagaauucauagucuatt

SEQ ID NO: 43 - DNA, 21 bp, Artificial Sequence
  Antisense Compound
  misc_feature (1)...(19): Bases at these positions are RNA
uguacaguaaguuguucgatt

SEQ ID NO: 44 - DNA, 21 bp, Artificial Sequence
  Antisense Compound
  misc_feature (1)...(19): Bases at these positions are RNA
ucgaacaacuuacuguacatt

SEQ ID NO: 45 - DNA, 19 bp, Artificial Sequence
  Antisense Compound
  misc_feature (1)...(19): Bases at these positions are RNA
ttatcgcttctcgttgctt

SEQ ID NO: 46 - DNA, 19 bp, Artificial Sequence
  Antisense Compound
aagcaacgagaagcgataa

SEQ ID NO: 47 - DNA, 19 bp, Artificial Sequence
  Antisense Compound
aaaatcagaagccaggtcc

SEQ ID NO: 48 - DNA, 19 bp, Artificial Sequence
  Antisense Compound
ggacctggcttctgatttt

SEQ ID NO: 49 - DNA, 19 bp, Artificial Sequence
  Antisense Compound
caaaaggtgcctgcacaag

SEQ ID NO: 50 - DNA, 19 bp, Artificial Sequence
  Antisense Compound
cttgtgcaggcaccttttg

SEQ ID NO: 51 - DNA, 19 bp, Artificial Sequence
  Antisense Compound
ttcagagaactcgaagaac

SEQ ID NO: 52 - DNA, 19 bp, Artificial Sequence
  Antisense Compound
gttcttcgagttctctgaa

SEQ ID NO: 53 - DNA, 19 bp, Artificial Sequence
  Antisense Compound
gttattccaggtcagcagc

SEQ ID NO: 54 - DNA, 19 bp, Artificial Sequence
  Antisense Compound
gctgctgacctggaataac

SEQ ID NO: 55 - DNA, 19 bp, Artificial Sequence
  Antisense Compound
atgaattctgcagggttgt

SEQ ID NO: 56 - DNA, 19 bp, Artificial Sequence
  Antisense Compound
acaaccctgcagaattcat

SEQ ID NO: 57 - DNA, 19 bp, Artificial Sequence
  Antisense Compound
tagactatgaattctgcag

SEQ ID NO: 58 - DNA, 19 bp, Artificial Sequence
  Antisense Compound
ctgcagaattcatagtcta

SEQ ID NO: 59 - DNA, 19 bp, Artificial Sequence
  Antisense Compound
ttatagactatgaattctg

SEQ ID NO: 60 - DNA, 19 bp, Artificial Sequence
  Antisense Compound
cagaattcatagtctataa

SEQ ID NO: 61 - DNA, 19 bp, Artificial Sequence
  Antisense Compound
gatgttgatcgggaagctc

SEQ ID NO: 62 - DNA, 19 bp, Artificial Sequence
  Antisense Compound
gagcttcccgatcaacatc

SEQ ID NO: 63 - DNA, 19 bp, Artificial Sequence
  Antisense Compound
aatgctgaagtaacagaac

SEQ ID NO: 64 - DNA, 19 bp, Artificial Sequence
  Antisense Compound
gttctgttacttcagcatt

SEQ ID NO: 65 - DNA, 19 bp, Artificial Sequence
  Antisense Compound
ttggtaatgctgaagtaac

SEQ ID NO: 66 - DNA, 19 bp, Artificial Sequence
  Antisense Compound
gttacttcagcattaccaa

SEQ ID NO: 67 - DNA, 19 bp, Artificial Sequence
  Antisense Compound
aagtcggaaaacaggttct

SEQ ID NO: 68 - DNA, 19 bp, Artificial Sequence
  Antisense Compound
agaacctgttttccgactt

SEQ ID NO: 69 - DNA, 19 bp, Artificial Sequence
  Antisense Compound
agtaaataagggcacggag

SEQ ID NO: 70 - DNA, 19 bp, Artificial Sequence
  Antisense Compound
ctccgtgcccttatttact

SEQ ID NO: 71 - DNA, 19 bp, Artificial Sequence
  Antisense Compound
attcaccattctctggatt

SEQ ID NO: 72 - DNA, 19 bp, Artificial Sequence
  Antisense Compound
aatccagagaatggtgaat

SEQ ID NO: 73 - DNA, 19 bp, Artificial Sequence
  Antisense Compound
aatgggactgagtaggtag

SEQ ID NO: 74 - DNA, 19 bp, Artificial Sequence
  Antisense Compound
ctacctactcagtcccatt

SEQ ID NO: 75 - DNA, 19 bp, Artificial Sequence
  Antisense Compound
tttatcttccacatcccag

SEQ ID NO: 76 - DNA, 19 bp, Artificial Sequence
  Antisense Compound
ctgggatgtggaagataaa

SEQ ID NO: 77 - DNA, 19 bp, Artificial Sequence
  Antisense Compound
aacagactaggtgttgcaa

SEQ ID NO: 78 - DNA, 19 bp, Artificial Sequence
  Antisense Compound
ttgcaacacctagtctgtt

SEQ ID NO: 79 - DNA, 19 bp, Artificial Sequence
  Antisense Compound
attgacataaagctccatg

SEQ ID NO: 80 - DNA, 19 bp, Artificial Sequence
  Antisense Compound
catggagctttatgtcaat

SEQ ID NO: 81 - DNA, 19 bp, Artificial Sequence
  Antisense Compound
tttattgacataaagctcc

SEQ ID NO: 82 - DNA, 19 bp, Artificial Sequence
  Antisense Compound
ggagctttatgtcaataaa

SEQ ID NO: 83 - DNA, 19 bp, Artificial Sequence
  Antisense Compound
actttattgacataaagct

SEQ ID NO: 84 - DNA, 19 bp, Artificial Sequence
  Antisense Compound
agctttatgtcaataaagt

SEQ ID NO: 85 - DNA, 19 bp, Artificial Sequence
  Antisense Compound
acctcagcaacaacagcac

SEQ ID NO: 86 - DNA, 19 bp, Artificial Sequence
  Antisense Compound
gtgctgttgttgctgaggt

SEQ ID NO: 87 - DNA, 21 bp, Artificial Sequence
  Antisense Compound
  misc_feature (1)...(19): Bases at these positions are RNA
agccaccccauugggagautt

SEQ ID NO: 88 - DNA, 21 bp, Artificial Sequence
  Antisense Compound
  misc_feature (1)...(19): Bases at these positions are RNA
aucucccaaugggguggcutt

SEQ ID NO: 89 - DNA, 21 bp, Artificial Sequence
  Antisense Compound
  misc_feature (1)...(19): Bases at these positions are RNA
aagucuuuuggaaaucugctt

SEQ ID NO: 90 - DNA, 21 bp, Artificial Sequence
  Antisense Compound
  misc_feature (1)...(19): Bases at these positions are RNA
gcagauuuccaaaagacuutt

SEQ ID NO: 91 - DNA, 21 bp, Artificial Sequence
  Antisense Compound
  misc_feature (1)...(19): Bases at these positions are RNA
ccuucauaccaugguucuutt

SEQ ID NO: 92 - DNA, 21 bp, Artificial Sequence
  Antisense Compound
  misc_feature (1)...(19): Bases at these positions are RNA
aagaaccaugguaugaaggtt

SEQ ID NO: 93 - DNA, 21 bp, Artificial Sequence
  Antisense Compound
  misc_feature (1)...(19): Bases at these positions are RNA
gagcaccucuaggcaaugatt

SEQ ID NO: 94 - DNA, 21 bp, Artificial Sequence
  Antisense Compound
  misc_feature (1)...(19): Bases at these positions are RNA
ucauugccuagaggugcuctt

SEQ ID NO: 95 - DNA, 21 bp, Artificial Sequence
  Antisense Compound
  misc_feature (1)...(19): Bases at these positions are RNA
gagcaaagccaccccauugtt

SEQ ID NO: 96 - DNA, 21 bp, Artificial Sequence
  Antisense Compound
  misc_feature (1)...(19): Bases at these positions are RNA
caaugggguggcuuugcuctt

SEQ ID NO: 97 - DNA, 21 bp, Artificial Sequence
  Antisense Compound
  misc_feature (1)...(19): Bases at these positions are RNA
cagugagacagaggcaggutt

SEQ ID NO: 98 - DNA, 21 bp, Artificial Sequence
  Antisense Compound
  misc_feature (1)...(19): Bases at these positions are RNA
accugccucugucucacugtt

SEQ ID NO: 99 - DNA, 21 bp, Artificial Sequence
  Antisense Compound
  misc_feature (1)...(19): Bases at these positions are RNA
agcccuuccacagcagcugtt

SEQ ID NO: 100 - DNA, 21 bp, Artificial Sequence
  Antisense Compound
  misc_feature (1)...(19): Bases at these positions are RNA
cagcugcuguggaagggcutt

SEQ ID NO: 101 - DNA, 21 bp, Artificial Sequence
  Antisense Compound
  misc_feature (1)...(19): Bases at these positions are RNA
aaggcuuauaccccucuuctt

SEQ ID NO: 102 - DNA, 21 bp, Artificial Sequence
  Antisense Compound
  misc_feature (1)...(19): Bases at these positions are RNA
gaagagggguauaagccuutt

SEQ ID NO: 103 - DNA, 21 bp, Artificial Sequence
  Antisense Compound
  misc_feature (1)...(19): Bases at these positions are RNA
ggaaaggcuuauaccccuctt

SEQ ID NO: 104 - DNA, 21 bp, Artificial Sequence
  Antisense Compound
  misc_feature (1)...(19): Bases at these positions are RNA
gagggguauaagccuuucctt

SEQ ID NO: 105 - DNA, 21 bp, Artificial Sequence
  Antisense Compound
  misc_feature (1)...(19): Bases at these positions are RNA
ggcuugaaggagcccuucctt

SEQ ID NO: 106 - DNA, 21 bp, Artificial Sequence
  Antisense Compound
  misc_feature (1)...(19): Bases at these positions are RNA
ggaagggcuccuucaagcctt

SEQ ID NO: 107 - DNA, 21 bp, Artificial Sequence
  Antisense Compound
  misc_feature (1)...(19): Bases at these positions are RNA
uacucuucucugagaugcctt

SEQ ID NO: 108 - DNA, 21 bp, Artificial Sequence
  Antisense Compound
  misc_feature (1)...(19): Bases at these positions are RNA
ggcaucucagagaagaguatt

SEQ ID NO: 109 - DNA, 21 bp, Artificial Sequence
  Antisense Compound
  misc_feature (1)...(19): Bases at these positions are RNA
ugaggauuuacucuucucutt

SEQ ID NO: 110 - DNA, 21 bp, Artificial Sequence
  Antisense Compound
  misc_feature (1)...(19): Bases at these positions are RNA
agagaagaguaaauccucatt

SEQ ID NO: 111 - DNA, 21 bp, Artificial Sequence
  Antisense Compound
  misc_feature (1)...(19): Bases at these positions are RNA
gcaggaccaggcagcucactt

SEQ ID NO: 112 - DNA, 21 bp, Artificial Sequence
  Antisense Compound
  misc_feature (1)...(19): Bases at these positions are RNA
gugagcugccugguccugctt

SEQ ID NO: 113 - DNA, 21 bp, Artificial Sequence
  Antisense Compound
  misc_feature (1)...(19): Bases at these positions are RNA
guccaggaacaggcucucutt

SEQ ID NO: 114 - DNA, 21 bp, Artificial Sequence
  Antisense Compound
  misc_feature (1)...(19): Bases at these positions are RNA
agagagccuguuccuggactt

SEQ ID NO: 115 - DNA, 21 bp, Artificial Sequence
  Antisense Compound
  misc_feature (1)...(19): Bases at these positions are RNA
uauacagguaauugucaggtt

SEQ ID NO: 116 - DNA, 21 bp, Artificial Sequence
  Antisense Compound
  misc_feature (1)...(19): Bases at these positions are RNA
ccugacaauuaccuguauatt

SEQ ID NO: 117 - DNA, 21 bp, Artificial Sequence
  Antisense Compound
  misc_feature (1)...(19): Bases at these positions are RNA
agacuucagggugcuggcutt

SEQ ID NO: 118 - DNA, 21 bp, Artificial Sequence
  Antisense Compound
  misc_feature (1)...(19): Bases at these positions are RNA
agccagcacccugaagucutt

SEQ ID NO: 119 - DNA, 21 bp, Artificial Sequence
  Antisense Compound
  misc_feature (1)...(19): Bases at these positions are RNA
ucuuuaauuaucugcgcgctt

SEQ ID NO: 120 - DNA, 21 bp, Artificial Sequence
  Antisense Compound
  misc_feature (1)...(19): Bases at these positions are RNA
gcgcgcagauaauuaaagatt

SEQ ID NO: 121 - DNA, 21 bp, Artificial Sequence
  Antisense Compound
  misc_feature (1)...(19): Bases at these positions are RNA
uguuaggccaacgucagugtt

SEQ ID NO: 122 - DNA, 21 bp, Artificial Sequence
  Antisense Compound
  misc_feature (1)...(19): Bases at these positions are RNA
cacugacguuggccuaacatt

SEQ ID NO: 123 - DNA, 21 bp, Artificial Sequence
  Antisense Compound
  misc_feature (1)...(19): Bases at these positions are RNA
uuaguuucuaggcucggcutt

SEQ ID NO: 124 - DNA, 21 bp, Artificial Sequence
  Antisense Compound
  misc_feature (1)...(19): Bases at these positions are RNA
agccgagccuagaaacuaatt

SEQ ID NO: 125 - DNA, 20 bp, Artificial Sequence
  Antisense Compound
ccgctgttctcaggtgacat

SEQ ID NO: 126 - RNA, 19 nt, Artificial Sequence
  Antisense Compound
ggagcuuuaugucaauaaa

SEQ ID NO: 127 - RNA, 19 nt, Artificial Sequence
  Antisense Compound
ccucgaaauacaguuauuu

SEQ ID NO: 128 - DNA, 550 bp, Homo sapiens
ctataaaaaaaaaaaaaaaaaaaaaaaaaaaaaggaaagccccgcgcggcgcgggccagggaagggccacccaggggtcccccacttcccgcttgggcgcccggacggcgaatggagcaggggcgcgcaggtaggatccggggcccgcgcgcggatcgggttgcgaaggtatcgcccgggcacgccggctgagggcgttcgggaagggctcggccgccggcggggaccacggggaccaccccgactccgagcggggcccgagcccgcgactctcggtgcgcgcggagcagcgcccggttccgtccttgccgccgaacggcagcggaggcgcgaggcccggggtgccttggcatctcccaatggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagct

SEQ ID NO: 129 - DNA, 31737 bp, Homo sapiens
tcttcaaaaaaaaaaaaagaatgattgacgtgcagtggcccctgccctgggaagccaaccctcaccctccaccacagcacttgatcccattgggcagtccttggcttccaccctcaaactgagagctccttgagggcagcagggggtcaaattactctctgggttctcagcactgcccagtgtgagtccaaatatctcccacctcccctgtctccgctctttcctcactcagggctccacagccatgccggccttcagcaattcctcctgcaggctgagctggttcccacagggcctttgcactagctcttccctctccctacacaagtcttcccccataggctcttcagagtctcagctcaaatggcctctactcaaaaagcctcttccagaccacctgccccactggtctaagaggtccctactttcttcttgggccctcctgacctgaaatactttgttgagtgttttccccccagtcatctggctgcatccgggttcttatggtgtccagacgttaatggtctatggggcctgtgttcgaatcccagctccacccctgctcctgcttacctgcagagtgattctgagtttctttcttttcttttttaaaattttataaatgtcaatttttgtagataggggtctcgctaacagggttgcccaggctggtctcgaactatggggttcaagtgatcctcccgcctcagcttcccaaagtgctgggattacaggcatgagccactttgcccaacccattttgaatttcttaaggactctgtgcctcagtttcctgatttgtaaaaccggtgatagtatcactcccttatagggtagttgccaggattgaacgagttaacatttgcagtgcccgacagattgtactagttactgattgaagggctgttttactatccaaatgtggctggagtaggagttgggtaaacatttattgaagaatgtgcaaccactctcacttggaagccgggctgttaggaaggggaggaggattccagtcgcccagccctcccccaccaaacgcaactgccccggcgcaaaagaggccgcggaggccaggcaggagcaggtcctggaggcctggtcggcgtgggcgttttattccgagaccaaggggatccactgcagagttctccgctgggcgtgacctcgggctacggcgtgggaggaagcgcgcggcaagacacccagcgaggtgctggggtcgcccccaggagaggacggcggctcggactgtccggcggcggcggcggggacagcgacaggggcgcgaggtggccgggacccgggccgggcgcgccgggcggggcggcgcatgcaaatctgccgggcgccggggcggggagcaggaagccggggcgggctgggtctccgcgcccaggaaagccccgcgcggcgcgggccagggaagggccacccaggggtcccccacttcccgcttgggcgcccggacggcgaatggagcaggggcgcgcaggtaggatccggggcccgcgcgcggatcgggttgcgaaggtatcgcccgggcacgccggctgagggcgttcgggaagggctcggccgccggcggggaccacggggaccaccccgactccgagcggggcccgagcccgcgactctcggtgcgcgcggagcagcgcccggttccgtccttgccgccgaacggcagcggaggcgcgaggcccggggtacgtggacacccagcgctccccaaagccggtgctggcagtgagacctccgccgggacggcctgcgggggtggggggcttggggttaggctgtcggaggccacgcagcccctcttctccgggcagtggcgcccagccctgcgctcaggaagtcagtgaggacttcggagagagaaagggtggggaaagttctgagaactgtaaatttgagtagtaggtcagtgaatcggggcggtctccgcctccgaatatcagatggacccaataatcgcgatcattgactgggacttgttttactgaaaggatgccaagtgaaacctcccactaacttctctgtggccggtgctgtgcgtcatggacattgtacagatgaggtcaccaagaccccagccgctttagtaacttgccctaaggtgccgcccacttgggagtccgtggggcagcaggactagagcccaggcaatcccacgccagagccagctgctctccatctctcaatcacgtttgaggagcccccacaataaccagaatctccgggagatttgcttaaaatgcagattcccaggtccttgccctgagattcagattcagtaaacccaggaatctgatctttattttatttttaaaaatttattttataaagatggtgtctcactatgttgtccaggctggcctcaagtgatcctcccacctcggcctcccaaaatgctgggatgacaggtgtgagccactgagccctgattttttttttaaacaaaatctggatttcttagacttaagcaagataggggaccactagtttgtctgcttctggggttcaaggaatgcccccaggccagcgctaaccctgctccagttctagtctctccccttccccagccctctgggaggcatgacccactgcacttccctgcccccagtagcctatgaggagcccaaggcatttgctgggacctcagtgcctttatcaaaatcccaaagtccaatggggaaacaggctcaggtgtaggtttggccagggtgtcctgcacattagggcattccagggtcctgcaggggtttcttagaactcggctagcccagcccatgaatgggaggtgacctgctaagccactcattcactcactagcactcattcagctcctgcgaggccctgggtggtgttctgggcactggtgcttgcctcaaggagcaccccctctgcaggaagacagacgtgagggctgggagggtgcggatgcaccagcggctgcaggaccacaagccagaggggatcagctcttcaaaggtcactgggccaggcttcccagcacccagggaccctgagctagccttgtgagtagagcaggggagtctggcaggtagaggatgggaggtaaagggaggcacagagtgggcggaggctgggaagtgtgaaagggcactgtgtacagaacctagggagtaagcagaagccagagatggatggaaagttgggctgggctgcattgtggaaagccttgaatgccatgccaaggaacttgggccagtgtattcaccttggcttgagttgtaaaagcaacaatgcagattcctgggccccaccctgggcctctggatctgaatctggagggagacgctggggaggcagcagggaggaaaacctgcagtcactcaccccatttacaacacatgcagttaccggcaggtccgagataacctaagggtttccaaatgctaggaagtgactcaatgactgatatgtttaatacaatacaatcccggttgcaaaatgcccagggcaagggtgggggtgttcagttttctaatgctcccagttcaaaaaggagctgaaatgaattgtcaaagaagtgacaagctgggcgcagtgactcacacctgtaatcccagcactttgggaggctgagagaggatcgcttgaacccaggagtttgagaccagcctgggcaacatagtgagatcctgttcctacaaaaaaaatttaaaaacttagccaggcgtggtagcaggtgcctctagccccagctatttgggaggctgaggtgggaggatggcttgagcccaggtggtcaaggctgcagtgaactatgattgtgccactgcactgtagcctgggtgacagagcgagactctgtctcaaaataaacaaatacataaatacatgagaagagagaaccttttctacctcccccatctccctgccctcttccacccaccccatctgggacgatggaggagagcgtttgggattagatgccccacctctcgactgccctcactggtacctgggagtgaggccaggacagaagccgcctaagtactgttcttcctcacatttgtaaaagtgaactttcttgagggttgatgacatgttttataagctacttcacacaatgtccaacacccagtgggtccacaatcaatgtatatgtctgttgttacctgtcaaaacgtatagctggggagtgacagtatggacttgagccagatcacccagtgcaaatgctggctttgctgcttactgtgtaactgacttcccagagcctcagtttctgcatttatgagattatagtgactatgccattgtagtgatgactaaatgagttaatatatgtcaagcatacaaaacagtacctggtatgtagtacaaactatgtatttgttaaatgaataccttttttttttttttagagccatggtctctctctatttcccaggctggagcacagtggcacaatcatagctcactgtagcatccaatacctgggttcaagtaatccttccacctcagcctcctgagtagctgggactacaggtgtgtgccaccacggctggctaattgtttttatatttttagagacaggatcttgctatgttgcctaggctagtcttgaactgctggtctcaagcagtcctcctgcctcagcctcctaaagtgttggatttatagatgcgagcaaacgtgcctggcctaaatgaataaatctttactgttgcctcctttgagctttattggttgggatttagtccactcattttgcaggtgagaactgagacccggagatgaatgcagtagccttgcctcaggtccacatctgagggacagactccatcctacttttcttcccatgtattctttcctgcttgtaccagttcactggggtgaccaagaaagacatttctgagttttcctcctggctggcacagtggagacatgcccagccacgtttagctagacttaccatggctgggctagaagagaggccaggagcttgtctggaggttgcacagacctgcccttggtgaatgtggatcggagcgccccggcagaggcttgaggtgctacagaggctgggggatccactgtcagaccgggtgcatcaccactgttttatgatatccagcaaattgctctgcgtctctgagcttaaggcagagagaagggcctgagcttgaagaggggatcccaggtttaaatcggagctggctacttcccagctctccactgtgtaatctggaggaagttgtttaacctctctgagcctccctttgttttgtcatgggtgtttgcagggagagcattcatgagatagtttgtatagacattttgcccttggaaggcgctcagtgattgctgtttttctctgcattttggaccgagtcccttctgcgtatccagcacctggcttcctcctgcatttcttcctcaggaatttattgagcacctatatgtgccagagcatcggaccaagtcctttgctctcctggggtttagattctagttgagcatacaggcaatgtacaaccaatacatatgttcagatcggcagctttatctgttattctgaaatccacgatttctgaaaaccgaaagtttttcataactcattttgaagctaaacttgaggctttttagagtcttcatcctcttcggtgtgatgctcagatgtctttctgcagagatattagtgcaattgatctttgggtgctttcccgccctgcatgatgggcatgtcggtgggtattttatacaaggtgacctttctacagcctgagtcatcctgaattctgaactatgtctggcccccaggattttacaggagagaacatggacctgcaatatcctatcaggtggagactgtgctaggaaggaaagcaaagctgggtgcaagtggaatcagggagggcttccctgaggaggtgacatttgagagggatgaggaagagagggccaggtggagggactcacagaggacggaagactccacttcctgactggcctgtgcttacccctcattctgcatgcacagttctgtctaaatgcgtccgttcttgcctttccaagaagtgctgatgtctcttacgtttcaagtctagctctgactgactcattctctctttaggtgcccccacaggcctcctcacctcttctcgtttgcttaagaaatctgaggtctgcgctaaagccagcctgggtgctgtaacctctcggccgaggatggtgcccagctgggcttgcatctggagttggaggaagtgggtcccgggaggggaagaggagccgtggggccttccaagggtttggtggaattgagttcctggtggggaggggctgtgttctgggtcttagagcctctcgcctgcaaagcaaagttgaacgtaacacgcgctcacatatctcgagtgccagcccggggctaggtgcttaacgcttgcttagccaaagtgccaggggctggggacacgggaatgagtgacccagaaatgagggccctcatggagctgaaagcattctgggcttggtgactttgaggaagtggcttttgaactgagacctgaaataccaaggcatgtggatatctggggaaagagtgatcctggcagtgggaacagagagtgcaaaggcccagaggcatgaatgagcttggtgtgtttgacccacagcaaggaagctggtgtggttggatggaaccaaacaagaaagaggtctaggagcagaggtcagggagggagctggggccagatacctgcattgtctcatttaacttgtgtaataaccctaccagataggagctactctcatgcaaatgttacagatgaagtaacagaggcacagagaggttcagtgacttgtccagggtcacacacctgaaggtggcagagcagggctgtgaacctagaagctccagagtcctcagtcctcaccatggtgtttaaacacaataaaatctatgcctgagtgctgaagacacacctgccatttagatcgatccacttttttacattgtcggtgtactccctgccatccatctgcccagaaatactgtgaatgtgggcagcgcaggactggttcctccaagccccattttacagacagtccaatcattttacatattattttatgaatgagcaccttcctcagtgtctcagaccaggggtccccacacagcagggccacacagcaggaggtgagtggcgggtgagtgagcgaagttttatctgtatttacagctgctccccatcgctcgcattaccacccaagctcggccacctgtcaggtcagctgaggcattagatgattctcacaggagcacaaacccttttgtgaactgcacatgtgagggatctgggttgcgtactttttcttttttttttaagacggagtttcgctcttgtcgcccagactggagtgcaatggtgcgatcttggctcactgcaatctccacctccccatgttcaagtgattctcctgcctcagcctggtggctgggattacaggcctgtgctatcacacccagctaatttttgtatttttagtagagacagggtttcactgtgttggccaggctggtgtcgaactcctggcctcaggtgatccacctgcctcagtctcccaaagtgctgggattacaggcatgagccactgcacgcggccctgggtgcgtgcttcttatgagactctaatgcctgatgatctgtcactgtctcccatcgcccttggatgggaccatctaattgcaggaaaataagctcagggctcccactgattctgcattatggtgagttgtataattatttcattatatgttaccatataataataatagaaataaagtacgtaatacatgtaatgtgcttgaatcatcccgaagccatggccccaaccacggcctctggcagtggaggctggagtccacatcacgctctgtgctgggtgcaggaatggagatgagtgggtgtcctgctcatttccctgaggaggaacctgaggctcaggctagagacttacctgggtgacacggctcctaagtggcagagtccagattcaaacccagggctacagacccagaagcctgtgcatttaaccacggttctgagctatccagcccaaagaggagagtttaaaaggattacatttcttcgttggaaatcagtgagcatagtcgcccagaaccatttttgaaaaactcagagaaggcctatgttttcctgaagcggccagggaagtattctgcctttttaaaagggaagactgaattctagggaggagaggcaccagctctctgggctgggcaggtctccaagctggcagggccacagcctcccgacggtcttgcctctagcatatttcttccagaagccacatttccacaggtgggcagctctaactgggagaaaatcttcctgagatttagccaaattagctttctttctttctttttttttttttgagatggagtctcactctgctgcccaggctggagtgcagtggcgcaatctcagctcactgcaatctctgcctcccgggttcaagtgattctcctgcctcaacctcccgagtagctgggatttcaggcacacaccaccacgcctggctaatttttgtatttttattagcaatggggtttcaccatgctggccagactggtctcaaactcctgacctagggtgatccgcccacctcagcctcccaaagtgctggtattacaggtgtgagccaccgtgcccagcccaaattagctttctaggagcttccactttctgagtctgtttctgcctcttggaggtagtgaccatctccctccaaactcggatacttctcttattcagactacacaactgtagttatttcaggtattctccctgactcaggaaattataagtttggcctgccttggcacatgagtcaactggttggagtcccacgcaaagactgtttgtttggtcaaagaggaaggagactattacctccttggatctagagcttatacttctgttgatgcaacctggaatcatactgtgtagcagccatatcccacttggagtgcatattgggcttgtcagctaaaatctccatgtttctcttctatatgagctgctgttaatccaagtttcttctattctgagagtccttgtctaaagctaagaggttccagtttttcatgctctctaataaatctacttcaggctaacatgctatcattttttctttgagtactgctttggtttctttccacaacttgttacacaatgttattcactgcccagtttagaatttcccttttggttcccattttaagacttatttaattatatgctttttaaatttttgagacagatagaattttttaagttatctttttattgttgatttctaaatttggtcatcatggttgttgtggtcaatatatgtggttcctataatatcagtggtagagaatttgctgagatctttataggattttctggtcatttttttcctcttgtgtttgaaaagaatgtatattttctgttgggtataaggttctagaaatgtatctatttgatcaagctagctattatattatctatatttgttatgtcactatgtggtttttttttttttacctgatttacctcccaccgtaatacaattacaattcaccataatagaggctttgttaagttctccttttgagtctgtcagttttttgctttatttttgaagctatgttgttaagtgcatagccatatgagtagaataagggttcaggatttttatacctttttggtgaattgttgcattttcccgagtagctgggactacagatgtgcactaccacgcctagctgatgtttgtgttttttgtagagatgggatttcgctatgtttcccgggccagtctcaaactcctaggttcaagcagtctgcccacctcagcctcccaaagtgctgggattacaggcatgagccaccgcagctggctgaattgttcctttaatcagtataaaagagccaacttccagcctgggcagcatagtgagaccccatctctaccaaaaactaaaaattagctgggtgtggtggcgtgcacctgtagttccagctacttgggagggcgaggtgggagaatcacttgagaccaggaattcaaggccgcagtgagctatgattgctccactgcactccagtctaggtgacagagtgagacattctcaaaaaaacaaaaacaaaaaaaccatccaccttctctcttctaatacttactcaagacatcattgtcatgaattctattttattgatgtcagtactcctgtactaataagccagatgagttaaaaacaacagcagccttgcaaccagccatgaagcaagtgatcttattctcatttcacagatgaggagactgaggctctgagtggactccagcctgccacggtctcccaccgagtcaggttggagcctgcacttaggcccagagttatgcttctgtgtctcagatgcttgggggtccctgcccacaaaggtggcccccatgtctgtgtgtccccacccatccaggaatggtatggggtgagacaacaggaagagctccatgggtagtccccacccactgccttccatatgccagtctgcaagcagaaaatgaacgggaagctcttacctgtgcccatgtgttgtcctggctttgggtttgcccagggggcaaagtcctggtcattgttgagacaagttcactgctgctgagggccgagcaaatggtgacaaaggccagattctaaattcctacctggttacttcctgctccgtcaccatcacctcgggcaaattctgaacctccctcagcctcattcctccatctgtgcttgcagggagaatattttactgtatcttccatagtgagctttccaggagaagcaaaaaagagtggtgtataaaaaagcaagtttatgactgcttctgtgccaggcactgtgctgtgcactccagagccaaaggcctgggttcaaatcccagcccactcactagctgtgcatccttaggcaggttcctgaccctctctgggcctcggcttctccatctctgcaatggagatgatattaattgtatctgcaatgctccttggattgggtccatggttaagtgagtgagtgaggctcaagggcttctttttttttttttagagatagggtctcactctgttgcccaggctggagtgcagtggcatgatcacagttcactgcagctgcgacctcgtgggctcaagccatcctcctgcctcatctcccttagtagctgggactacaggcatgtgccaccatgcctggcaatttttttttgtatttttggtagagacggggtcttgccatgttacccaggctggtcttgaactcctggactcaagtgatctacctgcctcagcctcccaaatggctgtgattacaggtaggagccaccgcgcccagccttcaagtgcttttttttgttagtgagactggtacaaccaatgccaaggctggtgagcaaagttgcacccagttggggaggggcgaaaccaagggcagaagggcccagggcatggcctctcatggctgaaggaagaggggacacgtggccagtcacagtgacctctgcagacctcaagaaggaggccagctaggagccatagctgtcactaagacagtcaaaatctaatcagcaccctggcgccaagaggccattgtccgctgctcaggacagtgagtgatagtcgataaccagctctctggaggtcagggccccgaggtggctgagatctgtgcctgcattaggacatttaaggggactccaagccaggatttgctagtgtttgcccagcagggagaggtggaaaattggggatgaccaaccaggcctccctgctcacagccaggggaattcatcctgtcgttcttccattcagcaaatattcatggagcacctgctgtgtgccaggcaccgtgctaggctctggggagatggctgtgagcgggatcgacactggccttgtgtgagctggccttgcagtaggggatatgagcaccaaacacataaggcccgagtgtgcaatatgccacaaagattgcagagaaataaagcaagataacaggttgcagaaagatggaggccaggctggctgctgttcctgggtaggtggcttctggacacagtttcctcaggctctgtttcctcctttgggcctctggaatcttctgaagacaggcctggcttccaggatcccggatggagttgcctgttgactgtgtggactcaaagtgtccacagtgcgccggagcgcgtggtcttccccagcaaatctgcttctcttctcggctctccatcgtgggaagggtccctgggatcctccccaccaccccagcctgaggccactttccctctgtccccgctctccccagcctgctttccccaccaagcgccaccaagtccctgctgcctcctcctcccaaacaggctcaaacctgccccctcttcgccagctctcagctcagcccgctcctctcctgcttaagagccttcactggctccccactgccctagagacagagtttgagtctactgccatggcttggcagacggtgttgtccccggcctgccaaccttccagctgccatcccagccagtccttctcccccttgctccagccaagggccccgcttgtagggtgggaagtgggccgtgccctccctcctgcctgcctattcgctgggtcttccctcattccctccctcaccccgccctctcataagaaccttcagaatctgccagacgcagtggctcacgcctgtaatcccagcactttgggaggctgaggtgggtggatcacctgaggtcaggagtttgagaccagcttggccaacatggcaaaaccctgtgtctgctaaaaatacaaaaaaattagctgggcgttgttgtgggcacctgtagtcccagctactcgggagactgaggcaggagaactacttgaacctgggagacagaggttgcagtgaactgagatcccaccattgcactccagcctggactacacagtgagactctgtctcaaaaaaaaaaaaagaaccttcagaatcataaaatgggtttaataattatacttatctcatagggagggcttaaagcacctaggccagtgtctggcaccaaggaaacactgtatacctgttaaaataagaataaaataaaatacacatgctcacttattgagtgcctgctgtatgtcgggcccctccccgagagaactctggtgcatccccgataaagaaacctgaccctctaggatgagggagggtcttccctgagcgcctgctaacagcctcctctgctaatctgaagtcctgcagcttgcccagcaggctggcttgtcacacgggctacccccacaggccgccatcttggaaaaggccaatggctgtggaatcattcttgactcctattagccttcctgttctctctcccgccccacagcatccatcctgtcagcaaatcctgtgggctctgtcttcaacacagatctagaattcagccccttctcaacctccccatctccgcagcctggtgtgagccctggcacctctctctggggccagtgtggtagcctcctcactgctacccacttctacccgtgcccccaccacccaagcaagagaggtcttttttaaagtctcatcatgtcacttctttgctcaaaacgctctggcaacttcttccttgctcaaatatttgttaaaagaagaaaggaatgaatgaatgaatggcctgtttgcatctgtaatagccagcacataggcagtacttggaaaacacccacagcaagaataaatgaggagatggctggacacggcagctcatgcctgtagcatgttggaaggccgaggcaggaggtttgcttgaggccaggagtttgaggttgcagtgagctatgattgtgccatagcactccagcttgggccacagagagagatccccatctctacaaaaatttaaaaattattcgagcatggtggtgtgtgcctgtagtcacaactacttgggaggctaaggcaggaggatcacttgagcccaggagttcaaggctgcactgagccgtgacggtgcgactgcattctagccacagaatgagaccctgtctctaaagcaaaaaagagaaggagaataagtgaggtacttgagcaagtgggtgaggatggttgagccacagcaggcccacagggaggctggtgggtgctgttggtggagggggccgatttgtgttcctggtggctgcagagccagtctgttgcctcccctccctcccactggctgggctggggacggagccaaccaggcagatcccgggccattctggggccctggcctggcctgccttactatcccagtggggccaccgacgtggctgccatttctggaaaccccagttctaggtaggttactgtcccaggaagaagtgcatgcctagcttcctgccagcccagcccagctgtcccagaagccagaggaagctctgggctggtttcttataagtagcatgactaactgattgcgccacaggagctgaggtttcaggccagtttctgtcgtggagtttcaggggtcagcggctccaaagccccagcccccacccgaggcccctgctgtccctggattcctctccacatgccacctgtcacagaggtgcttgcatcctgtctgcagaaccagcccctcctccaagccaggtttcctgcccagagggaagaagagcagaaggcctgtaccgccccctgtgcgcccccagtcctccgccccctgcccccctgccacccgctgtgggttcgggaagagtcctgctgggtcgcttccaggctgctgctggcttctgacatttcaaggcttaatcacttaatctcccccgagttttttaaaaaaagtgagtcaagtgtgttctcctggtggcgggcgtgttttcccaggcccatgcttctgctcatttcctggctgcggccttcactgactttgctgcttcctaactctgtgaccttggccttgtcacttgctgtctctgggtctcaggccaatgggtctcaggccagtggggctccccggacaaagttccagggttttggggactgcatttcaagggtcagcaaccacagtccttaaacacgtggattttggagtgacaatgcctaggttatggttctgtgaccaccagcaagtgctctagcctttttgtgcctcagtcttctcatcagtaaaatgggaatgttaatggcacctacctcatagggtcgttgggagaaccaaattgattaatatattgtaagtacttagaacagggccaggcacaaaataagtactgcatatacagttaactatggacaaaagcttgggacctggttcatgtttaaaaatgattattgaggccaggtgcggtagctcatgcctgtaatcccagcactatgggaggctgaggcaggcggatcaggaggtcaagagatcgagaccatcctggccaacatggtgaaatcccgtttctactaaaaatacaaaaattagccaggtgtgatggcgtgctcctgtagtcccagctactcaggaggctgaggcaggagaatcacttgaacccacgcggaggttgcagtgagctgatatcgtgccactgcactccagactggtgacaaagctaggctccatctcaaaaaaaaaaaaaaaaaagattgttgaatttattattgctgaatttttttttttttgagacagaactttgctctattgcccgggctggagtgcaatggtgtgatctcaactcattgcagcctctgcctcttgggttcaagcgattctcctgcctcagcctctcagcctcccgagtagctgggaatacaggcacccaccatcatgcccggctaatttttgtatttttgtagagatgtggtttcaccatgttggtgaactcctgacctcaggtgatccacctgcctcggcctcccaaagtgctgggattacaggcgtgagccactgtgcccagcctattattgctgacttttaaaccattcattcatttaatatatgtttattgtacacatgctatgcctgtgtcaggcactgtttcaggcactggggataaagcagtgagcacagcagacataaagctctgccctcatggggcttgtggtctctcagagggaggcaaacaatgcatttatagacaaatacataatttaaatttgaggttacctattatactcccaacttgaaaaactagcagtatattgaggtggctatttcacatctgatggaggaatatgtcttttgtgaaccatttatgttctttgcctatttttcttaaagggatgtttgtcttattgatttgtaagaactcttcatatattaaggccattaaccgttcataatgtagattgctacctttttctagtcttttgcttgctgatatagttgggatgtcatcctctctaaatctcttgttgaaatgtgatccccagtgttggaggtgggtcctggtgggagatgttgggtcatggggacgggtccctcatggtttggttctgtccttgccatagtgagtgagttctcacaagatctggttgtttagaagtgtgcggcacctccctcctctctcttgccctggctgtcaccatgtacgcctgctcccctcttgccttccccgtgaataaaagctccctgaggcctccccagaagccaagtagatacgggtgctatgcttcctgtacagcctgcagagccataagccaattaaacctcttttctttgtaaattactcagtctcagatatttctttatagcaatgcaaaaatggactaacacacttgagttctgtctttttcttatggcatattttaatctaggccagacacagtggctcatgcctgtaatcccagcactttggaggccaaggcgggtggatcacttgaggtcaggagtttaagaccagcctggccaacatggggaaaccctgtctctactaaaaacacaaaaaattagttgggtgtggtggcacacacctgtaattccagctactcgagaggctgagacacaagaatcgcttgaacccgggaggtggaggttgcagtgagccgacatcacgccactgctctccagcttggggaacagagcaaacctccgtctcaaaaaaaaaaaaaaaaatctagggaagttgaaaacattttgcagtcaaagctatctttgctatctgcatcttgaaataaaataaacattcatacatgtaacctcattaatctgattagagatagtgataagtactgggacagacataaacagggtgctgtgataagaagtcacagggagggcccgcattagttgaggggggtcagagaaggcttctgaagtgtttctggtgcaaatgcttggaaaagggagagctgggagtgtatcccagggctgaggccaatggaggattgcagaagagggagaggggataggaggggaatgttagagggataagcaggggaattggctcttttcttcattttcttaaattaaactttttattttcagataattaaagatttacacacagctggaagaaatcatagagaagccgggcgtggtggctcatgcctataatcccagcacttttggaggctgaggcgggcagatcacttgagatcaggagttcgagaccagcctggtcagtatgatgaaaccccatctctactaaaaatacaaaaattagttgggcatggtggcttgcctgtagtcccagctactggggaggctgaggcaggagaatcacttgagcctaggaggtagaggttgcagtgagccgagatcacaccactgcactccagcctgggcaacagagtgagactccatctcaaaaaaaaaaaaaagaaaagaaaaagaaaaagaaatcacagagagacccctgcttctatgctgttccccccgagggcaagattgtagaaatctgtaatacagtatcacagctgggatactgacactgatataagctactgaccttattgggatttcccgatttacttgtactcatttttgtgtttgcgtacgcatgtgtggtttagtgctatgcaattttatcacggcataggtttgtgtatgcagccaggatccagcacagttctatcatcacaaggatggctcaaattctccttttatagccacatccaactcccttcctgacccaccccctcagcccctggcacccactgatgtgttttccatttccatggttatgtgatttcaagtatgttacataaatggagtcacggggtgtgtactctgggaactggcttttttcactctagggcttggattttattctaagtcagagatgcttctctaatgagctgtgaaccccctggggatctgtggcccctgtgaagaagccctttaggagccagggggcattggatgtgtgagctggccctggtctcatttctgattttaaaatggctctggctgtgatgtggagaagaggcagggtgagcctcatctgcagcaactgctaactgcccacatccccccagacttctcaaaaatttgccactcccctgctcaaagaccctcaaaagctgcccactgcttagaggacaggcctttgacctgaggtcctcacttgggcttcagggattctgtgaacccctaaaatggaatgcagaattaagtgtatgcaccttgccagaaacagggtttggggtttacattagatgctcagataggtcattgatccccaaaacactaaggtctctgacttctagacagatagtagttaagaaccaaagctttggagcttgagtgctggatttaagacccatgactgtcacagctgtgcaacctcagacaaatcactcagcctttctgtgccttggtttcctcatctgaaaatggagaaaataatattacctatttcatagattattaaatgcattcttgtatgtaaaagatgtaaaagaaaacttggcatgtggtaaactctgtgtgtttaagtgaatggtgaggctgcccagtataggagggcagagctttccaggtaaagactttctaggcctcctgggattatcaggccctctctgtgttggttgttgtcatacatgacagcagtattccttccaaagggatgccaagttgtggttgcacactgcacttagatccagttgattgacagcactgttcaagtcaactgtgtccttactgatttctgcctgctggatctgtcgattaccaaaagagttgaagtaatagtaataatactattattatccaataatagtattattcagaataatcacacagatttctctatttctccttgctgctctcagtttttgccttacatattttgaccctttgttgttagttgcctacacattaaggattgttatttacatcttcttggagaattaactcccgaatcattatatagtgccacttttttttaatttaaaaattttattttttattttttgagacaggatctttctctgttgcccaggctggagttcagtggcatgaacacagcccactgcagccttaacctcctgggctcaagtgatcctcctgcctcagccttcccagtagctggggctacaggcatgtgccacctctaataacttttctttaatttacaatagtattagtttgttctcatgctgctctaaggacatgcccaagactgggtaatttataaaggaaagaggtttgactcacagttctgcagggctggggaggcttcaggaaacatattatcatggcggaaagggaagcaaagacatcctttttcacatgacgtcaggaaggagacacgccaaggaaagggggggaaagccctttataaaaccatcagatctcaggagaactcactcactgtcacgaaaacagcatgggggaactgctcccgtaatctaatcacctcccatgaggtccttcccccaacacgtggggattacaattcatattacaattcaaggtgagatttgggtggggacacagagccaggccatatcacattattattaattagtgccttctctctcatttttttgattcgcctaggtagagatttatcaatttcattgatcttttcgaagaaccaatttttgatttcattgattttcctcgattaattttctatttttaatttcattgatgtatgctccaattttgtatttttttcttctgcttgttttagacatatattgcttttctgtttctagtactctagatggaaacagattattgattttagaactttctttctaagatatacattcaataatataaatttccctctaagcactgcttttgctacgtctcaccgattttgataggctatattttcatttttatttagttaaaaatatttttagtttctcttgtgatttcttctttgatccatatgttattcggaagtgtgtttttaaatctccaaatacttggaaattttctggctttcttcctgctagtgacttctaatttgaatccattgtggcctaagagcatatgttgtgtgatttctaatgttttcaatctcttaaggttgttttatgtcccagaatgtggtctgtcttggtgattattccacatgaacctgaggagaatgtatattctgctgttgtgagatgtaacagtctataaatggatacctctgttaatatgacctcgggtgactctttttttttttttaagttgctgcttcaccattggcttttggagattttgcagatgataaaatcctcaggacttttctgctgctcagtcagattttcttatcctacacttgcggaactgttgtttgaaattgacaagcaggatggccaactctgcccggctcattttatttcatcttattgggttcagcctgcagttttaggagatgagattgaaggaactggtatactggaatccacggtgggctcggaagcaaacaaacgtgggttctgattctagctctgaccttttaccagctgtgggacctggtggatgggttatttggcctctctggcctcagtttccttagctagacagttgggataacagcctacctttcaggattcttgtgtggattcggtgaggtaatacttctaaagcacctagcaaagtgtcaagtacaaggtaagtgctgggcaagcagggattttgttcacacaaagccttgactcaacataaggaggctgtttccaatggctctgcttaactgagacctgagagtgattgctccatctgtggtggcatatgggctgaaactgcctagcagcatgtcagaagtggggtgttcgcaggacatccttgtccaggttggcagttgagcttggcaggtgaccgctgagaccccttccaactccaccacttcaggaatctcttttttttttttctgagacagtcttgctctgtcacccaggctggagtgcagtggctcaatcttggctcactgcaagctccgcctcccgggttcaagccattctcctgcctcagcctcctgagtagctgggactacaggcacccgccaccacgcccagctaattttttgtatttttagtagagactgagtttcaccatgttagccaggatggtctcgatctcctgacctcatgatctgcctgcctcagcctcccgaagtgctgggattacaggcgtgagccaccgcgcccagccaggagtctcttgatttagaatgggaacttgaaatcatctcctcttgccgtgtacttgaacgcgataacctggcacttatctttttaattcttttactcacctgcatctttgttctttttgcctctgatgttcagggaagcctcttctcccactgcatctgtgatctctcctgaaatgtagcacccacacatccctgagaagctagctgcggcacgtgtgtgtgcacatgggcatgcgtaagtctgggcgtgccttccacaacagattaaggatatcaatcctgacgccatgctgggatgttttagtgaatcttgacagggttgttgactcccctgtttccctggaggactttaccatgaaagtcttctgctgtgtgactttggacaggcgtcttggcctctctgtttctctttattcataattgaggctaaagtgacacctttgccatctctgccacaggatactgtgggcatctgtgtagttgtgagagtaaaagttactaggacttgccaaggaaatcagtgtcaggaacccaccaccccattcccgcccctcctttacttgagactttacttttgtaactctcagcaaggaagacgagttcaacctttgagccgatacagagccttcccctagagagagctttgcagcctcatgctgagccagttaggggccccacatcatgtccaaaatgaatttaatcaacaaacagttactgaacacgtcctatgtgccaggcaccgttgtagatactggggactcggcagtgaatgggacagaccctggctgtcaggatgacagacaatttaaaaagggacagatgtcccacgctggcttgtcctttgcacccctggcacttcgtgaggagcagcggccagttcttgtatatagactggcccttggtgtacatttgtctgacgtttccaaatgatgtatgcatccttggcaggaacccaggaaaagacatgctgtgctcttctcaggaattgtttcagggaggtgatgttggggatttgtcccattcttgttggtgagtttagatcacttggtgaaggtggtgtctgccaggtttctgtattgtaacgttcattgatatgtattttcactcattagttaatatacaatacaagttttgcagggagttactttgaggctgtcatagtaaaatagagctttcccttcttccctgtttatttatttattagagatgaagtctgactctgttgcccaggctggagtgcggtggtgcgatcatagctcactgcagcctccaactcccaggctcaagccatcctcctgcctcagcctcccaagaagttgggattacaggcacacaccacactgcacccggcctcctccatttattgatctatgtcaatacagactcctggactttaactccaagggttacaagtggataccataatattcattttgatgctcaaaatgtgcctgttttgtccaggtcaggctagctgctgagtacttccttattgtctggcacaagaattccaagcctatctggtacttttccttctcctggaatcagctatttctctaaggcgctctggttcctttcagtacagaatagtatttagaagccaagacctggtcactaggaaggcatgataatcaaatgtgatgaatgatcttggattgcatcctggacgtataaagggcattattgaggcaattagcaaaacataaataggggccgtggatgagatagtggtgatgcatcactgttaatttcctgatttcgatggttgtattgtgattatgcagagaatgtccttgtttttctgacatgcacactgaggtattggggtaatttattcattatggagatatgtgagtgtgtttatgtgtctcatacacagatgtttcttaatgatggggttttgtccccataaactcatcataagttgaaaatattattaagtcagaaatgtacttcatactcctaacctactgaacgttatagcttaacttagctcggaacacttgcattagcctacagttgggcaaagtcatctaacacaaagcctgttttgttataaagtgttgaatagcttgtgtagtttatggaatactgagagtgaaaaacagaatggtcgtatgggtgctccaagtactgcttctactgaacacccattgattttgcaccatcataaattaggggaagagaggaaaagcacgcgtaagggaggaagggccgggtacagtggctcacgcctgtaatcccagcactttgggagaccaaggtgggcagatcgcttgaactcaggagttcgagaccagcctgggcaacataatgagaccccatctctatcaaaaatacaaacacttagccaggcctcatggtgcacatctgtggtcccagctactcaggaggctgaggtgggaagattgcttgagcccgggaggtggaggttgcagggagtcgaggttgtgccactacactctagcttgggtaacagagtaaactctgtctcaaaaaaaaaaaaaaaaaaaaggaaagggaggaagggaaggtccctccaggtggaggggacagagagagaaaaggcatggaggtgggactgagtttggtgtgtttaagaagtgcaggtggcagccgggctcagtggctcacgcctgtaatcccagcactttgggaggctgaggcaggcggatcacctggggtcgggagttcaagaccagcctgaccagcatggagaaaccctgtctctactaaaaatacaaaattagttgggtgtgtggcccatgcctgtaatcccagctactcagaaggctgaggcaggagaatcgcttgaacctgggaggcggaggttgcagtgagccgagatcgcgccattgcactccagcctgggcaacaagagtgaaactctgtctcaaaaaaataaaatgaaataaaataaaataaaatgttaaaaaacaaaaaagaagaagtgtaggtggcaggcgtaggatgcatgggtgctgggcccaggtggagagggaggtggagccctgctggggggagggccttcaagtgtgcccaagtcactcggagagcatccagcaggggcctagagctcctgaggtggccttaagttcttatgatgccccagacactgcacacagtgcttcagaggcaacacctccgttcctgtttgctgcaagcctgtgaggttagccttggaaccattcctgtgccacagaaacacagccagagcaccaaaagagaaatgctgttaatggcaaagaactactcactagcagaacaaagcctgggagctgcttttttgaataagatcccaggagagagacccaagcgggaagtgaaggatgggcacttgaaatgttgcagggaatataacccagcgcgtgctgttttctttttttctttttcttttttttttttttctctttttgcaacagggtctcactgtgtcgcccagcctagagtgcagtggtgccatctcggctcaccacaacctccacctcctaggctcaagcgattttcctgcctcagcctcctgagtagctgggactacaggcacgcgccactaccgcccggctaatttttttttgtatttttggtagagacaggtttcaccatgttggccaggctggttgtgaactcctaaagtgctgggattacagatgtgagccaccacgcccggccccagtacacactttttggagggtatttgacgttatgtaccaggagtgctggggaaaagtacacacttgactccgtgagattctgcttcttagcatttttttcttttcctttttttttttttttgagacagagtctcactctgtcacccaggctggagtgcaatggcatggtctcggctcgctgcaacctccgcctcctgggttcaagcaattctcccacctcggcctcccgtgtagctgggactacaggtgtgcgccaccacacttggctaatttttgtatttttagtagagacagggtttcactatgttggccaggctggtctcaaactcctgacctcgtgatccgcccacctcagcctcccaaactttctgttttcttttcttttcttttttttttatgacagggtcttgctggagtgcagtggtacggtcttggcttactgcagccttgacatcccaggctcaaacgatcttcccacctcaacctcccaagtagctaggattacaggcatgcaccaccgtgcctggctgattttgtttaatttttgtagagataaggtctcactctgttgcccaggctggtcttgaactcctgggctcaagccatcctccctccttagcctcccaaagtgctgggattacaggtgtgagccaccacacctggcttctgaacatttttttcttaatgataatagctggtgctatgggctgaatgtgttcccccaaattcatatgttgaaacttcatcccattgtagtcatattaagagatggggcctttggagaagtgattaagtcatgagggctctgtggttggtcatgaatggggttagtgccctcatgaacgggctgaaggaagtgtgtttgccccttctgtcatatgaagacacagcagcatggcatcgtccgtgaggaatgggctctcaccagatctggatctcctagtgtcgtgatcttggacttcccagtctccagaactatgcattagaaaatttatttatttattatttatttattttgagatggagtcttgctcttgtcgcccaggctggagtacaatgacgcgatctcgactcactgcaacctctgcctaccccagttcaagcgattctcctgcctcagcctccagagtagctggaattacaggtgcccgccaccacactcggctagtttttgtatttttagtacagacggggtttctccatgttggccaggctgatctcgaactcctgacctcaggtgatccacccaccttggctccccaaagtgctggaaatacaggtgtgagctactgtgtctggcctgaataataaaatttaaaacaatttttcaaaaattcaccatgaggtctcactatattccctaggctggtctcaaacccctggactccaagtgatccaccccaccttcccgagtagctgggactagagatgcacaccattgcacccaatagagcaatacgtttctgttctttgtaaattacctgctctaaggtatttttgttatagcagcctatatggactaagctgacttgtaacgttacttgagactttaaagtgttccggtcactgttggagggctctgtctgtgttagctcatttaatccccacaacacctcaatcagatggggctattcttagtcccactttatagataaggaaactgaggcatggaagcacagcttgctcaaggttcacatctagtcagtgacagagcaggtatttaaacctcaggaaataatcagagaaacatgtgtagagggttgtccaaggaaggccacatccagaagcatctcccaggacagttgttgtgtagctcaccctctggactttgtgggtctgggtgttgtttcatgattatagagagagctctgtgaacgtggaggacctgttgtcggcagagacacaaatggccagggcatggctgggcagccgcagtggctcaggcctgtaatcccagcacttcgagaagaccagaggggcagatcatgaggtcagaagttcaagaccagcctggccaacatggtgaaaccccgtctctactaaaaatacaaaaattagccaggtgtggtggtgggcacctgtaatcccagctactcgggaggctgaggcagaagaatcgcttgaacccgggaggtggaggttgcagtgagctgagattgcaccactgcactccagccttggagacagagcgagactctgtctcggaaaaacaaacaaacaagcaaacaaacaaacaaataaatggccagggcaggggagggttgcatattgaataagatgagctctgctggaagcacaggtcagcactaacctgcttcctctctctctgcaggtgccttggcatctcccaatggggtggctttgctctgggctcctgttccctgtgagctgcctggtcctgctgcaggtggcaagctctggtaagtcaccacttctcaatcattcatttgttggctattaatggcgtgccagggtcctgcagtatgtcacctggccttatggagattacactgcagtgggaggggacagccaatgacaagtggccctgattatcagtaaattctaaagattgttagaaagtgatgggagccgggtgcagtggctcacacctgtaatcccagcacttcaggaggccgaggcaggaggatcgcttgagcccaggagttcgaggtcagcttgggcaacatagggagaccttgtctctacaaataataaaatattagccaggtgtggcagtgcacgcctgtagccccagctactcaggaggccgaggtgggaggatcccttgaactcaggaggtcaaggctgcagtgaactgtgatcgcgccactccactccagcctgcgtgagaaagtgagaccctgtcaaaaaaaaagagaaggtgatggggaaagaacacagaacagcataagagggggttggggaagctgggtggagtgggggggattgcagttgaaagtagggaagtcagggaaggcctcattgagctgacttggaggaagcgggaaccgtgcagatgtctggggaaggctcattcttggcagagaggccctgcactgagcctggcgggagggttgagcacaggagggaatgtggtggaggagagtgagcagcaggagggagcagtgaaggtcagcaaggtgacagagtggctgaatcaaaaaagaccttgcagtgtttgagcagaggatccatatcatccattatgttccaaaggactcttcaggatgccgtgtggagaaaggaagagggtggaagccaggaggtctggagggaggtctggagtggaggagatgagaggctccggatccctctgggaggtagatttgaggacagattggaattgaggtgaaagacagagaaagagaagtggccaggatgactccaagatttctgacctaaactactgggaaggacgcggttgtcatttctgaaatgcagaaggatgccagaagagaaggtactttggggaggggcgggaatcaggagttagttttggacatgagataagcttggaatatttatttgctatctaagacagctccttaacatggtaagcccttatgcaagttgttgtcagctgagatgggcgtggcactgagcatgggagcatggaggcgcctgagtggtctcatgctcaggtggtttagcaaactcagtgtacatcctgccaattccagtcctgccatggccactgacaagctaggagggcgctgaaaggagaaggaccccgatgtctcctccagcccatccatctcctctctcccattggccaaacccaaccggaaactaaaggccaagggtacccggtgatgaagactgtggtatcagcctcctgagcacagagagggcagaaaggggtggagacaaagaggggcgcagatagtgggcaaatggggaagtggcacttcccctagctcgagggcagaggcttggtgtgatggaatggcactccttaaactgctacatattttccctttaatttggccaagaacaagttgtcaagtttgtgtgagataaaggtgcacttggttcgttcttgtctaatggcccccgcacccatgggtatttcttcagcttccacagtcatcccgacactagctgggaagctccagcagccctggtcctggccccagctctgtgggcgctggccctcaactttgcctgcactgtgcttttgtgctattccccttggtcctgtttgggtgcaagtccccctcacgcattgagttcctgggccgctcaggctgctcctgtgtctccccagggaacatgaaggtcttgcaggagcccacctgcgtctccgactacatgagcatctctacttgcgagtggaagatgaatggtcccaccaattgcagcaccgagctccgcctgttgtaccagctggtttttctgctctccgagtaagcctgcgctggagctggaggtttggggaggttgtgcccaaagggtttgccccaagagtgagctgggtccaggtggtgcgctggagtgcaggatgctgagtatggtttgctgctgtttatatggtgttagaggggaggtcccatctccagggacatgttatgtaagatacagtggagcgcatggtgggagtgttggtccacgtggcacatggatacggctggaatactggactagaccagcagttctcacactttttggtctcaggaccctttttcacacttaaaaatgagtgaggacccaaagggctttggtgtaggtaacacatcattctatgtttacctaattagaacttgcaatgaagaaatggtgtaatttttaaaaaattaaaacaattaaaaattttttttcttactgaaatggaggtctcactgtgttgcccaggctgctctcaaactcctgggctccagtgatcctcctgcctccgcctcccaaagtgctgggattacaagcgtgagccgctgtatccggcccaaaatggagaaattttaagtcccaacaacatgcaagcccgcattcaacaaatcttcagatcaattacatgatcacaggtcatgtagcctctagaaaattccactgtacgccagtgagagagagtgaaaaggcaaataacgtccctgtattatgatgaaaagagttttacctggtgggcccagaccacactttgagaaccactggactagacccttgattgaggagtacggtgttgagagtggagtcctctgtgatggtggatggaccaggacacatggcataggagtcaggtggttccctgggctactccatggtgcacaggatgcttcgttacactggtgcccaggacataatcacgtacacaagacacacagttacggggcagactggggatatacggcacaccagcatgcagcgttcaccagtaaaggtggtattccatgattattctaaggtagatgggctgtgctttgtttccattggcttagtccagggattggcaaactatggcccgtgagccaaatccggcccactgcttgtttttgtaaataaagttttattggaacacactggctgctgtagttgtaacagaaactgcatggccctcctttatgttttttgtttgtttgtttgtttgtttgttttctttgagacagagtttcgctcttgttgcccaggctggagtgcagtggcacaatctcggctcactgcaacctctgcctcccgggttcaagcgattctcctgtctcagcctcccgagtagttgggattaatggtgcctgccaccacacccggctaatttttcgtatttttagtagagaccggttttcatcatgttggccaagctggtctcgaactcctgaactcaggtgatccacccgcctcagcgtcccaaagtgctgggattacaggcatgagccactgagcccggcctcctcctttatcttaattgaaataattcagaaatggaaagtcaaatactgcatgttctcacttataagtaagagttaaataatgtgtacacatgggcattattccatgtaccatggaataacagacattgaagacttgggagggtgggagaggggtgaaggaagagaagttacttaatgggcatagtgtacaccatttgggtgacggacccaccagaaccccagacttcaccactaggcagcatatccagtgagaacagatctgaggcttgccatcaaaattgcacttgtaaggccgggcactgtggtggctcgcggctgtaatcccagccctttgggaggccgaggtgggcagatcacttgaggtcaggagttcgagaccggcctggccaacatggtgaagctccatctctactaaaaatacaacaattaactgggtgtagtggcgcacacctgtaatcccagctactagggaggctgaggcgggagaattgcttgagcccaggaggtggaggttgcagtgagccgagatcaca

SEQ ID NO: 130 - DNA, 21264 bp, Mus musculus
tcactgtactctagcctgggtgacagtgagactttgtctcaggaaaaaaaaacaaaaacaaaaaacaaaaaactcgtaccccctaaatttatacaaataaccaaaaaaaaaaaaaaaaaaggaaattgtgtggcctttgaagtccaaaatattaactatctggcctgttacagaaaaagtttgcagacccctggcctagcccgtgagatgtgggttggctgttaaggtggaacattggaattatcttacgatggccaaactgtgcgatgcagagcttatgttgttctaaattaattagtgccaccggttcttccctttcatgggctttcaggaacaagctaagtcccaggaccagggccggcagctaggcaggtgtgaggagcatccttggtgcatgtggtaagaggctgtggccagcaagagaggcaaccctagtcggctgccccagcacaccctggccgctcccaagcccccagatctgtcctcacatccgtgatcgggaagctggaagagtctgatgcggttcctggaggcatgtcccggacacagctgtggggcccagccagcctacaggtgaccagcctaacccagcccctgtgtctgcagagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatggtgagcagggcggagtgcggcaggggtggctgggtgtgttcccacagctgcctgggctgagggtggggtgggcaggggaggaggtggggtcatagcaacagcaggaggaagccgcctgtattttcccaaatctgatgggattcctgcccctgcctgggcctcagtcctcccacctttgaaacggagctggtcgcagtagaccaccaagcccccttcagcccagctgtttccacccctgaacttaagtgcccaggaaggcgtattgagatgaggtgtgcttgctggaaggcatgcctgctgctgattgaaaaccgaactgggaacattccttccattctgtgtccactggtcagctgctgcggctttggatggtcttgaccgtggaaggctgaccttcttctggtacccggagtccctgcaggaatcccccttgagcttgctgggctgtggtgacaggagtttaaaacatgcgttgtattccagtgatgcatgatatgacatgcatcacaggaataaaaacctgaggtctcatggatatgattgcttcaaaggagaccaagttttaaaacagatgaatcaaaataaagaaaaatactcagtaaatcatcataaagtacagagatgtggccaaaggtgtgaaggatgcagctgtaaaagctgaagtttgaggccgggtgtggtggttcatgcctataatcccagcactttgggaggccgagcccagcggatcaccggaggtcaggagttcgagaccagcctggacaacatggtaaaaccccgtctctactaaaaatacaaaaaattagtctggcatggtggcaggcgcctgtaatcccagctacttgggaggctgaggtaggagaatggcttgaacccaggagaaggaggttgcagtgagcttagatcatgctactgccctccagcctgggcgacagagtgagattacgtctcaaaaaaataaaaataaataaaaataaaaagattttttaaaaggctgaagtttgggttactttggctcatacactttgccttcactgtagaaaggtggttagtaaagaccaggcgcggtggctcatgcctggaatcccagcactttgggagcccagcgcaggcagatcacttgagccctgggctattgaggctgcagtgagctgggattgtgccactgcactccagcctgggcaacagagtgggaccctgtctcaaaaaagaagaaaaaaagggtaattaataaacactaaagttctatgtagaattttagcaacattattgttattataatcttctttgctatggctctgaatctgtgtggtgctccagaagtatgctatggaggttttgtcgaccaaaaatctgggtggtggctgtggtttgtaggccggggctgggctgggtgatgggggagtcactgcatagatcctcacatagaggccgcttctcccgcagtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatgtgagtgggcatgctttgacgtttttctgtgacctctggggaacagggtgggtgaccagcagaggcccagtccctggagccaggagcctgggaggcaagccctggggctggatagcaaatcccaggagctagagacctggcttctcacctggctctgcactaggcaagtccctttgcttcctggccccccacccctcacatcagagaaggggagttatctctgcatgccgctcctcctctgtaaaggtagggctgtgggccacatctgtgtttcccagtttgggggacacaagtgatcgtaggtggcacattgacagctcacttgaataaccctattattgaagagaataatactgactcaagagacagtgacccgtgtcagttcccttttgaggccaacgggttaaggaggaagtccccatacagctgactcgtttactaattcctcttaatgaagagagcagaggccacaccccaggcttagactttcccaagaaaacaagatcagtttgttggttgttccccatggaagctggtcctgacattcccttcacagtagtgttggtggagtttttgttgttgtttgttttgagacagagtctcactctgtcacccagggtggaacacagtggcgtgatcttggctcactgcaacctccgcctcctgggttctagcgattctcctgcctcagcctcctgagcagccgggactacaggcacctgccaccgtgcccagctaatttttgtatatttagtagagatggggtttcactgcgttggccaggctggtctcaaactcctgacctcagatgatccactcgccttggcctcccaaagtgctgggattacaggtgtgagccaccgcacctggccagtggagttccttcttaagtacatgtattgacatctttaaaaagggcgagaggatttacaggaaactatcaggtcagtaatggcaggggccgtccacagtgggtggctgagtccccctatttttctgctggtgtgcagggaggtcatttcctgccacccatgtttccccaccctgaatccaccttcctcacattcccattggagggacaatctctggacatatgggacctggggtcccacagggctgcaatccaatgcctgctgtgccactcgccagctgtgtgatgttgggcatatcccataacctctttgtgcctcagtttcctcatctgtaacacaggagtgacaagagcacccgcccacagggctatgacagtacaaggtgtgtgatacagatgagctcccctgtttggcccacatgtgtcctaaaagccatgtgccctttctcttgagtgccccaggccacagagatccccatctgcccgctgtcccacacactggtctgtcatttgttccttgaggtttgtgagggccggctctgtgcatcccaggggcccaggctgggcctggttggctctcagggagcaggcacccgccaccttaagctcccatgctggtgtctgtcactgcttcctctcaatctggccaagccaggggtgtcgatttatatctctcaggtctggtttcccctttggcactgggccaggtatggggaaagagcaggaatggggcagttggctcacacagcagaggctcagaaagcggggggcatggggggaaggagtgcacagatgctagagagtggggcaagttttgtttggtcaataaatctccttctcatgccccaggcctgtgcaagacctacagagagtcccaaggatgggctggggggaagagaaaggtaccaccttcagagtccaaagatatgttatttaatattttcatatttctagatctgccttcaggcatggctggatccagcttctaggaacctgtccagctctgcgccctgctttattctgtactggcttcgtttttaggcaggctcttccctcatgtagtggcagatatgcctactagttgctccaggcctacatcccaaagccacagtgggaaaagggttttttttcttgacggttctaataagagtcctaaggctgctgctcagtggcctggcttcgatgctgtgccagcctctgaaccaatcactggctgtgggtggagagagggtgctggtggagggccctgcttgtccagggaggagtcacatacctgcctctagggctgcaggtgggctcagctccatccaaaccagatgaactgaaaataaggcaggagtggcttccccaggggaaactggggaagaggaagcaggactgtgctggctaaaatgccagccaggtttaagacgtggcaccagatgccagtcatgggattggattggtcagcatgcctgggctatggcttaggggtatgttggtgctcagggatgccacaggcctccagataccaggtctgaggcagaagaatgaagtccagcttctcttgtgggtggaacagtggcaactgagataccccatctctcccttcccaagaacagagctgaacataaagaatttagtgattggccagagcttggccacatgctcccctctgatgaatgataggccaggtgatgggattggcacaattggcttagactaatgagggttggccctggagttgcaggcagtggagttctgtcctaagcagtgggcacctaaacccgatggcataaaagctgggcgggtgtccacctgcatctgccacagcactataggcaccaactgtggctcatactgagtgggataaattccagaaagaaacattaggaacttactatagaattttggggctagagctactcattcattcccctagataatttctaggcaaggttccatagtggagggggagttttggcttgggcattgaaggatgcataggagttttctagatggggaaagaagggaacggtagaccaggcagagggaactgcatgataaaaggtttatgggtgtgaaaattcatggaatgtttgaggattatggggttgggggatgtgggaatatgtgtagcgataaagcaccaaacaaagccaaaagtttagttagagccctgaatgcctgcctcataatggtttccatattttatatgcctactatgtgccaggcacattgctcagggtcacacagctggaaatggcagggctgagtttttgttgttgttgttgttgttgagacagagtctcactctatcacccaggctggaatgcaggggcgtgatcatggctcactgcatccttgacttcctgggatcaggtgattctcccacctctgcctcccaggtagctgggactacaggcacaggccaccacgccaggctaattttttgtatttttagtagcgacagggtctcgccatgttgtccgggctggtctggatctcctggcttcaagtgatccccctggctcagcctcccaaggtgctgggattacaggcttgagccaccgcatccagcccagatctgagatttgcacccagtatttgaactcccaagcctgtgctctttttcctcccatggacatttctctcagagatggtctcccaaacacctgtccttcttgttaaaaaacagacaaaccgcaagtagttctttggaagctcagatttctcttttgtttcttagtaaaacatttcccagttcccagctcccttccagggtgtaagatttcttcggtaacttacatctagctgttgcttcttgtttgctcatgtttagaaagaaagacaaaagagagtgagaattttctctcccttccccagtctccccacaactcacaccccaccctcagctccctctgtaataggaaaatctctgaactctctgtagttgctccagcaatcttttggaactttgcttctttcttgtgaaaaaacctccccttggctcactttgcaccaggtttccccaaatgtgcttccaaccacaagcagaaatggagctgccagtaaccaggaagaaactgccgggggctgaggaagaggagagggaggtgcatagccctggatctcgcagggagaggggtgacaggatgagaactcaggttgctcacttgccatcagggtcagtcatgaatatagcgttcatgtatcactttttaaagcttttttggagggtaaaagtaatagttacacaaaataaaaatacaaatggtacaaaaggacttagaatggaaacatgtttctctcccgactccagcctcctgtttttcttcccagagactgaccactgctgtctgtctcttgccagaagggaaagggaggcaaggttagggcaggcagagggcatgtgcatcctttagagagagcttatgtctatacaagcaaatgtgtgtgttcagtcatcgctgtcttagttttctattgctgcataataatggtactaccagcttcacagctttaaacaacacccatttattatctcatagtttctgtggttgggagtctggacatagcttagccaggttctctgctttagagtctcgtgaggctataatcaaggtgtgggatggggctgcagtttcatctgaggctcaattggggaagggtcacttctaagctcatacaatattggtgacattcagtccctggcaggctgttgaactgagagcctcagtttcgtgctggctgttggttgtagttaaccctgaattccttcccatgtgccctttgcaaagccatcaaggcagagagacttgcctagcaagtaggatattacagtcttctgtaatataatcacatccatgaaatcctctatatatcccatcacctttaccatattctgtgggttagaaacaagtagcaggtcctgcccacactcgagaagaccagatgacacaaagatgtgattcaaagtggggatcatcggggccatcttaggtttgtctgcagtgatcactgtgccatctctctctctctctttttttttttttttttttccgagacgaagtcgtcactctgtcacccaggctggagtgcagtggcatgatctcagcttaccacaatctctgcctcccaggttcaaatgattcttctgcctcagcctcctgagtagctgggattacaggtgcccgccaccacacccagctaatttttgtatttttagtagagacagagtttcaccatgttggccaggctggtcttgaactcctcacctcaagtgatccacccacttcggcctcccaaagtgctgggattacaggcatgagccaccatgcccagccccatctctctttaaaaaacaaacaaacaaacaaaaaacataaaaagaagcagagaacacatacacatctgcatcttcccttgtttacttaacaatagatcttggaagtcacttctcagtagaggctaggttgggcagagcattggattctaggccagtgagtttggacttgaccatggagacactaggaagcccatgaaggacagagagagatgcctcgaccctgccagtcctttagaaagatcacccagtgctttttgtataccaaaccctatttgaaatacttacgtatattaacccatttccttatcaccacaaccctgcgggaagggagataggcacttttattatcttcattttgcagatgaggacattgaggtccagagaggttatgtcacttacttaaggtcacacagccaggaagtggtagtagggactcttacccttgttttacagatgagattgaattatctcacgaaaactcagaaaggttaaacaacttgcctaagtaacatacagctaattagtcgaggagcctgacgcatgttgctgtagcctggtcacagttacagaggtggcaagcaatggcctgaacaggacgaacaaccaaatacccaggctggtggctcttaaacatggtggggtcagctaacgacagcaaccagggtgggcactggtgcccctcgcccccggctggtgccctaacatctcccttttctctaccagttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactgtgagtatcaagaggcctaagcaatggtaatctccactctccattcttcccctgtggccagacacttcccctggctgagtctctgggcttttatatcataggatgcctctaatggcaatcctgccattagatacacctgccgtggtgtatctgccaggtaggcaggctaggctgcagtaacaaacaagcccacaatttccatggcttaacactatgggaatatatttcttgctcacgtaacaagctaacgtgaatgttgctggtttgtaggtggtttccctccctgtagaaatctggggagtgaggttctttccatcttgtggtgccgtcattctccaggacaaagattcttacctacttttgtgtcctggtttcctttggcagcctggtgaagcctatggacctcatttcagaatatttttaaatacataaaatcccagcctgggcaatatagtgaaacccccatctgtacaaaaattagccaggcatggtggcatgcacctgtagtcccaggtactgggaaggctgaggtgggaggatcacttgagcccaggagtttgaggctgcagtgagccgtgatcgtaccactttactcccacctgggtgacagagcaagagcccatctctaaaaataaataaatacaatgaaataaaataaaataaatagaactacagaggaaactaattgtattgaaatgcagttataaaacatttaaacacatttttaatctagagatatatgtgcttctttattaagatctataaataataagttctaggggtagctcgcataaatactgtaatttcaaagtagataagcataaataatactttatgatactgaaattgtgatgtgatatgagaatagctgtgagttttgttttgctggggacaggatcagtgatgctgtcattactggggtctcttccctccattctttttttaaaattgtattttattttatttttaaaattttaaaataaatagagacagggtatcactatgttgcccaggctgcttttgacctcctgggctccagtgatcttcccatcttggcttcccaaagtgctgggattacaagtgggagccagtgttcctggccccttcctccattcttaatggaaggagatgctaggtgtgagaggttagggaaagtaaagatgtaatttctttcccatccaagttctcagacccctgaattctacctgcagccatgttggtccatcaaccccaagtgaagaatccctgctctagggccccaccattgtctgtatccagccagcagaagaggcgtgattatggagatcacatctgcttcttgaaagcagacagcccggaagtgggccgcatcacttcctctcaaattctattggtgaaaatggtcacatgactacacatagccacaaaggaggctgggaactttctcacttggaacctacatcccagaaacaactcttttcagtgaggtatcccacaggtctttcgcagtagaaatattgattatctcacataaaatgaagtcttacaaatggacctactgggttttgtacagcagccaagtgatatctcttcccttctgctgtcttcccttctgccatccttcacatggtggcattgtatccttagacttgccacccatgccctcaggttggccgttgcacactgtcttacataaagcaggaaggaaaggaaaggctgctacgagagagtgtaccttgtgcatctcttttttaatcaggaagcaaacatctttctagaagcttccctagcaaaattccccttacatctcattggccaagactgttacatgttacatggttactgttattacttgctcattgcaaggaagactgggaactcaaatgcctggaaaaaggaacaggataatcgtgattggctcaagccttagggtgggcatggctccctgacaagggagagaggaaaaagctgttgagtgaagaagactgcttcagtttccccatctgtataatgggaggagtaagggctgtcgtaaaaactcaatgaaagaagattcttcaacgtggtaggtgcagtggcagctggcagtaccctgaccctgccaccgcacagccctctcagcattgctcatcctgcactgtggatatcagttgagccacgtgtctcctgccctgggctgtgagctccataggcagggtctccatggctgtatctccagaacccagcacagaaccaggtgcttgggaaagttttgaattgattctcatctgccattggcatggggaagggaactagcttgtatgaaacagataacaatgtatgggaccctcattcattatttcagcaaatatttgctgagttcctcctacatggctagccctgtgctagacactggggaatcggcgatgaacaaagcagatagaaatccccactcttgtggagctgacattctggagggagagacaaaaagcaaacatataaagaaagaaagaaatcacatggatctggatgacagtgagtgctgggaagaaaataaaagcagaggaaggggatggagcgatgggcagggggcaacggtagggagggtgtcggggaaaactttttggagaatgtgacgatgaaagtgaacaaggagaagtcaaccgtgttgagatgatggcagctaatgatgtggacaggccactctgttctgagtgcattatctattgattcatcatgtcatcctcgcaacagccctgcacgatcaattctgtcattaaccccatagtacagatgaggatgcggaggcacagagaagataagggacttgtcctgtgtcacacagcaaggagccatccggctcctaagttggtgcatttgacttctgtgcttccggaaagaaagagcagcaagtttaagatctggaggtggcactgagctttggaggagcagggggcaatgaggtggccggtgtgacgaggactcaatgtgcaagagggagagtggtggggagatgaggtggaggggtggtcggcggtcagatcgtggagggtctcggacgagggtcctgaccctgggtctccagtcctgggaagtggagcccaggctgtaccatggctgacctcagctcatggcttcccctcccacttccagcctacagggagcccttcgagcagcacctcctgctgggcgtcagcgtttcctgcattgtcatcctggccgtctgcctgttgtgctatgtcagcatcaccaagtgagtcctgggcccagtgctgccgagcagtccctctggagtgcagggtggcagggacttgcccctctagtctgcccctttgcagtcctctcagtcaataatacgcatttactgagcagctactacacaccttgagagtagagctgagaacatatcgacaaggaccccacttttttctttttttttttttttttttttttttgagacggagtctcactctgtcacccaggctggagtatagtggcacaatcttgcctaacagtaacctccgcctcccgggttcaagcaattcttctgcctcagcctccagagtagctgggattacaggcgcatgccactatgcccggctaattttttgtatttttggtagagatggggtttcaccatgttggtcaggctggtctcgaactcctgacctcatgatctgcctgcctcagcctcccaaagtgctgggattacaggtgtgagccactgcacccaaccaggactccacatttctaaaaccggcatcctactggggagactgaaaatacatatcaatcacaaacaggtggttttccatagtgacccactctctgaatgcactagaccagggtgcaggccagagatcttctggggtgctttttgcaagggggaccaggataaggctctccaaggagggaaaatttgaggggggccctgactggggagaatgagctggccagggataagcaagatggagtcatcccacatccccttacaacgctgggtgcctgggcaactgggggcatctgggggcatgtggtaggagccagaggaatttgcgacgattgccctgatggagtcaggagacctgggtttgaatcctggccttggagcttggtagctggcggccgacaagttgctgaaacccctgagcctggggttcctgctttgcagagtgacagtgatggtgagaacatatttcatcagccagaagaggccaaatcacagtaaaggctgagggaggagatgagtggcgagtggctgggaggtggtggaaggagcctcgtttccagagagctcttgccagcccttggaatcatggtgtctcagagcctcagtcctcccatctctgaaatgggactagcaagctcaacctcactaagtcaggattagaggtggctaaggattattaacatgattgatgaaagtgcccactcttggcccagcacacactaggtaggcagggaatgcaaattcccctccatatcttgtcactgatgcctccgagcaaccttggactgatcgccttgctctgagcctcagtttccccatcacctgtacctcttcccactccccatcactatatcccagcatgccagcctctttgctgttctttgtctttggtttcttgttttgttctgttttttagacagggtctcactctgttagccaggctgaagtgcagtggcgcggttacggctcactgcagcctccaattcctgggctaaagagatcctcccatttcaacttccagagcagctgggacaacaggcgcttgccaccacacctggctaattttcttattttaatttaatttaattttattttttgggacagagtggagtctcaaaaaccaagctggagtgcagtggtgcgatctcgactcactgcaatctctgcctcccgggttcaagcgattctcctgccttagcctcccgactagctgggattacaggcgtgtgccacgacacccagctaatttttgtatttttagtagagatggggtttcaccatgttggccaggatggtcttgaactcctgacctcaagtgatccacccacctcgttctcccaaggtgctgggtacaggcatgagccactgtgcctggccaattttcttacattttgtagagactggctgtcacttatgtagcccaggctgatcttgaacttctacccctttatctttattcatggcacttattaccatgaatgaatgacctcatataagcatttctttcgtttttttttttttttctttgagatggagtctcatgttgtcccccaggctggagtgcagtggcgcgatctcagctcactgcaacctccgccttccgggttcaagcgattctcctgcctcagcctcctgagtagctgggattgcaggcgcctgccaccatgcctggctaagttttgcatttttagtagagacggtgtttcaccatattggccaggctggtctcgaacttctgacctcaggtgatacacctgccttggcctcccaaagtgctgggattacaggcgtgagccaccatgcctggcctcatataagcatttctgtctccatttatcatccatctttccctcttgaaggtcagtttcaccaaggcaggcatctttgtctcgttcactgttgtggcctcagggccaggcacagtgagtcaaacatagaaggtgctcaataaatatgtgtttatttattgaaaccatgggcagaggctaattcagaagcggtctgaggaccttacctcccagtgatgatgcaccatggccccaggcaggccaggaagagagaagggttgtgtttctccgtaggtcccccagcttcccaggccatcccaggccattccctggtcatttgccctcagctgctctgaaaaagggattgttgaggggaacctagaatcctctctctgcagtttgagtctttcctaatcccctggggtctcattcccactgaggacataggtggcctcctcaggaactctgtgctgggtaacagaatgcgggagtgtgaacctggctctgccacctaccagctgtcactccacctccttgggcctcactctcctcatctgtagaatagggttagcaatagaatccatgtcaccaggttagaatgatgagtcagtggtttgacctccagaaactaatcagcctgatctctgatgccaaataagtattggtgataacgaccacttttatgggaggagcgttcacctgtcaataattcagagatcaacaccttttccttttgtttttcaggattaagaaagaatggtgggatcagattcccaacccagcccgcagccgcctcgtggctataataatccaggatgctcaggtaggagtaggcgtggatgaggacatgtgggactgtgtacatgaagaagtgtggttcagaacacctgggctgttaaggaccttcactggcttctggaatggcaaatagacagtcaggagggttgcaggggagacagagacagaagccgaatgaggtcattagcagaccagaggctttcccgcccttccccttggcaatcccagcctggggtgggcttctctggggttggtttcctgtttttttccctccccttgggagaatgacccttgggtcatcatcactgtgtcattccctggggaggtgccagtaccagggctagaggccagaaggagtggaggaaggagagggtgacaggctttctgtgtcttcttcttaagcataggaaactgcccccgaagcactagcaaatcccttccgggttctcattggcctgaaatgtatcccacccctaagccaggggtggagtcagcttccccaaggcgatggtcctgtgggtgagtgggtggggtttgcctgagcaagatgagagttctctaggtaggagaaagggggattataggtcctgtctagaagagaaggtctgagggtccttgcttttccagggactctggaatctagtggtgttggctttgaatcctgactctgccactcactggcagtgtggacttgagcaagttgcttaattctctgagcctcagtttcctcttgtgggttataacagtgtttacctggtaggacagatattggaatttattgagacaatacatataaagtgcatattccagcctcttgcaaataccaagtgccatttatgtatcagttagtgtttgctgtgtaacaaacgaccccgaaatgtagagggttacaacaactttatttagcttatgcttctgcaggctggcatttggggctgggctcagcagtgagggtggcgggggaggctgggctgggctgggctgggcagatctgaattgagctgacccgtccccgtagcctccctccgtgtctgacagttggctttttttttttttttctttttctgagacggagttttgctcttattgcccaggagtgcaatggcgtgatcttggctcactgcaacctctgcttcctgggttcaagcaattttcttgcctcagcctcccaagtagctgggattacaggcatgtgccaccacgccaggctaattttgtatttttaatagagatggggtttcttcatgtcggtcaggctggtctggaactcctaatatcaggtgatccacccacttcagcctcccaaagtgctgggattacaggcgtgagccactgcacccagcctagttggctgacttttacctgggacagtgcaggtgcctgagccatgtgcctctcactctccagcaggccggcccaggcttgtttacagagtggctcagttttcaagggtgggaagtcccaaggcttcttgaggcctaggcgcagcactggcatgatatcacttccatcacattctatgggcccaagcaagtcccagggccagtgtagattcaagggatgggaggagattcagagcactcctctgtggccacttttgccatcgaccacagtccctgtaaatattaggacaatgtaattaattcccaggaatctgaagctcagaaagcgtaagtgacctgttggacttctgatctgtgtgatgtcgaggcttgtaccccttcctgagcattgccgtactccaggccgggctgcaaggccactctgctctttcattggctgtctctgtattttaggggtcacagtgggagaagcggtcccgaggccaggaaccagccaagtgcccgtatgtatctgaacttaggtcacagcctgcatgcattgggaaggtgatagaattggagaggcaagcccctagctccatgtctgccttctcttccctgcattcggtaattgccctgtgacattagccttcaagggacggcaggaggaggggtgttctggaaacgtggactgctggccaagccccctgagtttcactggtgtgtcaggtacatggtgataccccttgggagtgctgttatagttaacaaccagagcagccgtgcctgttgttaaaatcttgacctaattgtatacttgtcggcaaatagccactatcctgaacactcccctccttttttttaatatacaggatctcactctgtggcccaggctggtgtgcagtggtgcgatcatagctcactgcaccttcaaactcctgagctcaagtgatcctcccatcttagcctcccgagtagctgatactacagatgtgcattaccacgcctggctattttaaaaggtttttgcctgtaattccagctactcaggaggctgaggcatgagaatcacttgaacccgggaggcagaggttgcagtgagcgcagattgtgccactgcactccagcctgggcgacagagtgagactcttgtctcaaaaaaaataataccaaaaaaagtttttgtaaagacaagctctcgctgtgttgccccgccactgtggcctccttagcttcttccctggggcctgctggacctttccatactccagaaactaaagggggtccaggaccctgcttcaaccctaggatcccgcatcttttttttttttttttttttttggacgcagggtcttgctgtgtccctcaggctggagtgcagtgattcactgcagcctcaaactcgtgggctcaagtgattctctagcctcagccttctaagtagctgggactacagtcatacaccaacatgcccagctaattttccttttttttaattcttgtagagatgtttgagacggcttgggctctgttgcccaggctgttctcaaactcctgagctcaagcgatcctccctcctcagcctcctaaagtgctgggattacaggcgtgagccaccgcacccggcttccatatcctttctaattggtcatggcttgggataatggtgttgcttttaattatcatcatccataaagactttttcttactcaacagatctgagcttgtatttggtgcccaggacatgtgctgggttcccgaaatcccaaagacacagaccctaccctcagggatttctcattctagcaacatagactgatcaattactgattataacgttagaaggcatgtctgaagtagacagccatcaggacatggtgatttcaggctgggctttgaagaatgaataggagtttttcaagtgtcgaaactgaaccctgaccaacctttgcttttgcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctcttaggtgcatgtcctcttgttgctgagtctgcagatgaggactagggcttatccatgcctgggaaatgccacctcctggaaggcagccaggctggcagatttccaaaagacttgaagaaccatggtatgaaggtgattggccccactgacgttggcctaacactgggctgcagagactggaccccgcccagcattgggctgggctcgccacatcccatgagagtagagggcactgggtcgccgtgccccacggcaggcccctgcaggaaaactgaggcccttgggcacctcgacttgtgaacgagttgttggctgctccctccacagcttctgcagcagactgtccctgttgtaactgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgttttagtttcatcacctgttatctgtgtttgctgaggagagtggaacagaaggggtggagttttgtataaataaagtttctttgtctctttattttttatgtattaaccaaacatacctccagacactgctgtgagtgctgtgtctctgttaactcctggaattcacccatccagaggaaccaggatgcaagaggttaagaaacttgccatctgggtttgggttccccatacaaggattcaaatagttgatttaggaagtaatcccgggaaaccctgctaaggtagtggggaactgaggcagggaaggacacaaaccaagaaagtgttacctgaaaggggtccagatgcagaccccaaaagagggttcttgaatctcatgcaagaaagaattcagagcgagtccatagagtcagtgaaagcaagttaatgaggaaagtaaaggaataaaagaatggctactccgtagacagagcagccctgagggttgctggctgcctatttttatggttattgattaattatattccaaacaaggggtggattattatgcctcccttttagaccatatagggtaacttcctgatgttgccatggcatttgtaaactgtcatggcgctgttgggagtgtagcagtgaggacaaccagaggtcactcttgttgccatcttggttttggtgggttagagccatcttctttactgcaacctgttttatcagcaaggtctttatgacttgtatcggtgacgacctcctgtctcattctatgactaagaatgccctaacctcccaggaatgcagcccagtaagtctcagcctcattttacccagcccctcttcaa

SEQ ID NO: 131 - DNA, 6649 bp, Mus musculus
  misc_feature 1081, 1082, 1083, 1084, 1085, 1086, 1087, 1088, 1089,1090, 1091, 1092, 1093, 1094, 1095, 1096, 1097, 1098, 1099, 1100,1101, 1102, 1103, 1104, 1105, 1106, 1107, 1108, 1109, 1110,1111, 1112, 1113, 1114, 1115, 1116, 1117, 1118, 1119: n = A,T,C or G
  misc_feature 1120, 1121, 1122, 1123, 1124, 1125, 1126, 1127, 1128,1129, 1130, 1131, 1132, 1133, 1134, 1135, 1136, 1137, 1138, 1139,1140, 1141, 1142, 1143, 1144, 1145, 1146, 1147, 1148, 1149,1150, 1151, 1152, 1153, 1154, 1155, 1156, 1157, 1158: n = A,T,C or G
  misc_feature 1159, 1160, 1161, 1162, 1163, 1164, 1165, 1166, 1167,1168, 1169, 1170, 1171, 1172, 1173, 1174, 1175, 1176, 1177, 1178,1179, 1180, 1407, 1408, 1409, 1410, 1411, 1412, 1413, 1414,1415, 1416, 1417, 1418, 1419, 1420, 1421, 1422, 1423: n = A,T,C or G
  misc_feature 1424, 1425, 1426, 1427, 1428, 1429, 1430, 1431, 1432,1433, 1434, 1435, 1436, 1437, 1438, 1439, 1440, 1441, 1442, 1443,1444, 1445, 1446, 1447, 1448, 1449, 1450, 1451, 1452, 1453,1454, 1455, 1456, 1457, 1458, 1459, 1460, 1461, 1462: n = A,T,C or G
  misc_feature 1463, 1464, 1465, 1466, 1467, 1468, 1469, 1470, 1471,1472, 1473, 1474, 1475, 1476, 1477, 1478, 1479, 1480, 1481, 1482,1483, 1484, 1485, 1486, 1487, 1488, 1489, 1490, 1491, 1492,1493, 1494, 1495, 1496, 1497, 1498, 1499, 1500, 1501: n = A,T,C or G
  misc_feature 1502, 1503, 1504, 1505, 1506, 1645, 1646, 1647, 1648,1649, 1650, 1651, 1652, 1653, 1654, 1655, 1656, 1657, 1658, 1659,1660, 1661, 1662, 1663, 1664, 1665, 1666, 1667, 1668, 1669,1670, 1671, 1672, 1673, 1674, 1675, 1676, 1677, 1678: n = A,T,C or G
  misc_feature 1679, 1680, 1681, 1682, 1683, 1684, 1685, 1686, 1687,1688, 1689, 1690, 1691, 1692, 1693, 1694, 1695, 1696, 1697, 1698,1699, 1700, 1701, 1702, 1703, 1704, 1705, 1706, 1707: n = A,T,C or G
  misc_feature 1708, 1709, 1710, 1711, 1712, 1713, 1714, 1715, 1716,1717, 1718, 1719, 1720, 1721, 1722, 1723, 1724, 1725, 1726, 1727,1728, 1729, 1730, 1731, 1732, 1733, 1734, 1735, 1736, 1737,1738, 1739, 1740, 1741, 1742, 1743, 1744, 1937, 1938: n = A,T,C or G
  misc_feature 1939, 1940, 1941, 1942, 1943, 1944, 1945, 1946, 1947,1948, 1949, 1950, 1951, 1952, 1953, 1954, 1955, 1956, 1957, 1958,1959, 1960, 1961, 1962, 1963, 1964, 1965, 1966, 1967, 1968,1969, 1970, 1971, 1972, 1973, 1974, 1975, 1976, 1977: n = A,T,C or G
  misc_feature 1978, 1979, 1980, 1981, 1982, 1983, 1984, 1985, 1986,1987, 1988, 1989, 1990, 1991, 1992, 1993, 1994, 1995, 1996, 1997,1998, 1999, 2000, 2001, 2002, 2003, 2004, 2005, 2006, 2007,2008, 2009, 2010, 2011, 2012, 2013, 2014, 2015, 2016: n = A,T,C or G
  misc_feature 2017, 2018, 2019, 2020, 2021, 2022, 2023, 2024, 2025,2026, 2027, 2028, 2029, 2030, 2031, 2032, 2033, 2034, 2035, 2036,2239, 2240, 2241, 2242, 2243, 2244, 2245, 2246, 2247, 2248,2249, 2250, 2251, 2252, 2253, 2254, 2255, 2256, 2257: n = A,T,C or G
  misc_feature 2258, 2259, 2260, 2261, 2262, 2263, 2264, 2265, 2266,2267, 2268, 2269, 2270, 2271, 2272, 2273, 2274, 2275, 2276, 2277,2278, 2279, 2280, 2281, 2282, 2283, 2284, 2285, 2286, 2287,2288, 2289, 2290, 2291, 2292, 2293, 2294, 2295, 2296: n = A,T,C or G
  misc_feature 2297, 2298, 2299, 2300, 2301, 2302, 2303, 2304, 2305,2306, 2307, 2308, 2309, 2310, 2311, 2312, 2313, 2314, 2315, 2316,2317, 2318, 2319, 2320, 2321, 2322, 2323, 2324, 2325, 2326,2327, 2328, 2329, 2330, 2331, 2332, 2333, 2334, 2335: n = A,T,C or G
  misc_feature 2336, 2337, 2338, 2541, 2542, 2543, 2544, 2545, 2546,2547, 2548, 2549, 2550, 2551, 2552, 2553, 2554, 2555, 2556, 2557,2558, 2559, 2560, 2561, 2562, 2563, 2564, 2565, 2566, 2567,2568, 2569, 2570, 2571, 2572, 2573, 2574, 2575, 2576: n = A,T,C or G
  misc_feature 2577, 2578, 2579, 2580, 2581, 2582, 2583, 2584, 2585,2586, 2587, 2588, 2589, 2590, 2591, 2592, 2593, 2594, 2595, 2596,2597, 2598, 2599, 2600, 2601, 2602, 2603, 2604, 2605, 2606,2607, 2608, 2609, 2610, 2611, 2612, 2613, 2614, 2615: n = A,T,C or G
  misc_feature 2616, 2617, 2618, 2619, 2620, 2621, 2622, 2623, 2624,2625, 2626, 2627, 2628, 2629, 2630, 2631, 2632, 2633, 2634, 2635,2636, 2637, 2638, 2639, 2640, 2848, 2849, 2850, 2851, 2852,2853, 2854, 2855, 2856, 2857, 2858, 2859, 2860, 2861: n = A,T,C or G
  misc_feature 2862, 2863, 2864, 2865, 2866, 2867, 2868, 2869, 2870,2871, 2872, 2873, 2874, 2875, 2876, 2877, 2878, 2879, 2880, 2881,2882, 2883, 2884, 2885, 2886, 2887, 2888, 2889, 2890, 2891,2892, 2893, 2894, 2895, 2896, 2897, 2898, 2899, 2900: n = A,T,C or G
  misc_feature 2901, 2902, 2903, 2904, 2905, 2906, 2907, 2908, 2909,2910, 2911, 2912, 2913, 2914, 2915, 2916, 2917, 2918, 2919, 2920,2921, 2922, 2923, 2924, 2925, 2926, 2927, 2928, 2929, 2930,2931, 2932, 2933, 2934, 2935, 2936, 2937, 2938, 2939: n = A,T,C or G
  misc_feature 2940, 2941, 2942, 2943, 2944, 2945, 2946, 2947, 3112,3113, 3114, 3115, 3116, 3117, 3118, 3119, 3120, 3121, 3122, 3123,3124, 3125, 3126, 3127, 3128, 3129, 3130, 3131, 3132, 3133,3134, 3135, 3136, 3137, 3138, 3139, 3140, 3141, 3142: n = A,T,C or G
  misc_feature 3143, 3144, 3145, 3146, 3147, 3148, 3149, 3150, 3151,3152, 3153, 3154, 3155, 3156, 3157, 3158, 3159, 3160, 3161, 3162,3163, 3164, 3165, 3166, 3167, 3168, 3169, 3170, 3171, 3172,3173, 3174, 3175, 3176, 3177, 3178, 3179, 3180, 3181: n = A,T,C or G
  misc_feature 3182, 3183, 3184, 3185, 3186, 3187, 3188, 3189, 3190,3191, 3192, 3193, 3194, 3195, 3196, 3197, 3198, 3199, 3200, 3201,3202, 3203, 3204, 3205, 3206, 3207, 3208, 3209, 3210, 3211,3362, 3363, 3364, 3365, 3366, 3367, 3368, 3369, 3370: n = A,T,C or G
  misc_feature 3371, 3372, 3373, 3374, 3375, 3376, 3377, 3378, 3379,3380, 3381, 3382, 3383, 3384, 3385, 3386, 3387, 3388, 3389, 3390,3391, 3392, 3393, 3394, 3395, 3396, 3397, 3398, 3399, 3400,3401, 3402, 3403, 3404, 3405, 3406, 3407, 3408, 3409: n = A,T,C or G
  misc_feature 3410, 3411, 3412, 3413, 3414, 3415, 3416, 3417, 3418,3419, 3420, 3421, 3422, 3423, 3424, 3425, 3426, 3427, 3428, 3429,3430, 3431, 3432, 3433, 3434, 3435, 3436, 3437, 3438, 3439,3440, 3441, 3442, 3443, 3444, 3445, 3446, 3447, 3448: n = A,T,C or G
  misc_feature 3449, 3450, 3451, 3452, 3453, 3454, 3455, 3456, 3457,3458, 3459, 3460, 3461, 3591, 3592, 3593, 3594, 3595, 3596, 3597,3598, 3599, 3600, 3601, 3602, 3603, 3604, 3605, 3606, 3607,3608, 3609, 3610, 3611, 3612, 3613, 3614, 3615, 3616: n = A,T,C or G
  misc_feature 3617, 3618, 3619, 3620, 3621, 3622, 3623, 3624, 3625,3626, 3627, 3628, 3629, 3630, 3631, 3632, 3633, 3634, 3635, 3636,3637, 3638, 3639, 3640, 3641, 3642, 3643, 3644, 3645, 3646,3647, 3648, 3649, 3650, 3651, 3652, 3653, 3654, 3655: n = A,T,C or G
  misc_feature 3656, 3657, 3658, 3659, 3660, 3661, 3662, 3663, 3664,3665, 3666, 3667, 3668, 3669, 3670, 3671, 3672, 3673, 3674, 3675,3676, 3677, 3678, 3679, 3680, 3681, 3682, 3683, 3684, 3685,3686, 3687, 3688, 3689, 3690, 3791, 3792, 3793, 3794: n = A,T,C or G
  misc_feature 3795, 3796, 3797, 3798, 3799, 3800, 3801, 3802, 3803,3804, 3805, 3806, 3807, 3808, 3809, 3810, 3811, 3812, 3813, 3814,3815, 3816, 3817, 3818, 3819, 3820, 3821, 3822, 3823, 3824,3825, 3826, 3827, 3828, 3829, 3830, 3831, 3832, 3833: n = A,T,C or G
  misc_feature 3834, 3835, 3836, 3837, 3838, 3839, 3840, 3841, 3842,3843, 3844, 3845, 3846, 3847, 3848, 3849, 3850, 3851, 3852, 3853,3854, 3855, 3856, 3857, 3858, 3859, 3860, 3861, 3862, 3863,3864, 3865, 3866, 3867, 3868, 3869, 3870, 3871, 3872: n = A,T,C or G
  misc_feature 3873, 3874, 3875, 3876, 3877, 3878, 3879, 3880, 3881,3882, 3883, 3884, 3885, 3886, 3887, 3888, 3889, 3890: n = A,T,C or G
gaattcatgctgctttctcggcctagttctgctgagcttctctgagcacctgagccaagcagggctggtcccaagcaccatcacaggccagcctgtttctgtcacatatcgatgtatctcacatagtcccctctcccctgggcaagggtcatacaatccagtcacatgaagacaagtggccagccaggagggatcaaaagatggcttgcagactcagaagacagcaagatgtggtggcttgtatctgcaatcctacttgggaggctgagacaggattactgtgacttcaagttcagactgtgctacaatatgacagccaaagctgtatagcaagatcctgtcacaagaagcaaaaaactaagccaacccaatccataatgctcaaaacaggatggcaagcccatgggatggcttagctagcaaaggcaaggctggtcacagttgcgctgctgtgggcgagagaacaacttcgtgatgtcagttctttcttttcacttttgcgtggtttctagggatagatcttgcactctcaggcttgcgggccatctcatcaacccttaagcaagtttcctaacagatccatgcctcagcttcttcatctgaaaagggatagttgcgcatgactccctcttggtagtagcagcaaggttcctgcctgaacctagcaggagacactcgctcttaattgatgagttgttttgctttctggcacagacgctgagtaaacattgggaaaatgagcaacagccgcaaacacaagccaagctggtctgtagggaaagggcaaagattcgagtcccgcagtcgtgggacttaaaagctggagctctggacagctgcggacgcgtggagtggggcatgcaaataagccagcgatcaggcaaagtccggaggactgcgggtgaaaatgagctgggtgaggggcggggcatgtaaatgaccgtgcacggggtggggcgtcgcatgtaaataagggacaccaggcggcgtaggaagcggggcgaggtccttctcgcaggaaagccccgcgcggcgcgtggagcctgaactcgcaggtaggaacgggcggtgggaaagcctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatggctcattttatttcattgttaggttctggctggacttctcgaagctgaggagaagcagagggacctggcttctgattttggatctgcgtgcttgctggttctggggcctgctggtcttgttcctgtaacctaggactcggggcttgcacatgctttttttttgaagttgctggagagggagcccaggaccttgtgcgggtgagccaggtatcctgacactgcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatctgcccactcttctttctgcaggcaccttttgtgtccccaatggggcggctttgcaccaagttcctgacctctgtgggctgtctgattttgctgttggtgactggatctggtaagtcactcattcattcatcaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatctggccggttctgtctctgcagggagcatcaaggtcctgggtgagcccacctgcttctctgactacatccgcacttccacgtgtgagtggttcctggatagcgccgtggactgcagttctcagctctgcctacactacaggctgatgttcttcgagttctctgagtaagtggggtagggaggcagcaccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtaacagagtttctatgtctgcagaaacctcatatgcatcccgaggaacagcgccagcaccgtgtgtgtgtgccacatggaaatgaataggccggtccagtcagacagataccagatggaactgtgggctgagcacagacagctgtggcagggctccttcagccccagtggtaatggtaagacctgtctgggtgctccagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacccaggtgcccttcctccccagtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaagtgagtgggcgccacggatccctgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcatatgtccctgtcttccttcagttcattgtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaaccgtgagtatcagggtcgtaggctgtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtacagcgcacattgtttttttcagcaagtaatgaaaatctgtgactgagtgaccttgggggctgcggtggtgaggagagctcacgggaatcctggagcagtgtagctggcgtgtcaaaagcagaaacgcaggagatgggtcagtgcgccgaggcccggagtttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttcatgactgtttttctccttgcagacttccagctgcccctgatacagcgccttccactgggggtcaccatctcctgcctctgcatcccgttgttttgcctgttctgttacttcagcattaccaagtgagttcctgctttggctggtgtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaacctacttcctttccaccctcaggattaagaagatatggtgggaccagattcccaccccagcacgcagtcccttggtggccatcatcattcaggatgcacaggtaagagggtacaggcgttgcatagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccactgattggctgtctattttaggtgcccctctgggataagcagacccgaagccaggagtcaaccaagtacccgtatgtatctgaacttgaacttgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttaaccctcagctttacctctgcaggcactggaaaacttgtctagacaagctgctgccctgcttgctgaagcacagagtaaagaagaagacagacttcccgaaggctgccccaaccaagtctccccagagtcctggaaaggcgggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcagggagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggcccagcgggcacgataactggcagtcaaggcgatacctcgagggaactgtggggctggtcctggttggtggtcaggtggtagggatagcagatggcagactttggtgagtgagtgagtctgactgtgttctggaagatgggaccaggctcagcactgtctgctcacgtccccactgttgcgacacctagtctgtttgcaaggaggacaggacaggtcacatggagctttatgtcaataaagtctttatcttgtcaggtttcctttactatacacacgccgagcccacgtgcacgacagttgaaatgtgcaggcagggggttggggaagtggggagacaaggccccagcagttggtttaagggaatgacttgggaatggggcagagctgtggctactccatctctcatccttaccctcctgctaccacagcttgtgcccatgtgtgcctgctcaggggaggggtcctcctctgtccctctcctttaggcagagtgctagaggtgttggatgctccttagcacgcagaggccgtgacactggctgc

SEQ ID NO: 132 - DNA, 496 bp, Mus musculus
tcgagagcggccttctcgcaggaaagccccgcgcggcgcgtggagcctgaactcgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgccgtggactgcagttctcagctccgcctgcactacaggctgatgttcttcgagttctctgaaaacctcatatgcatcccgaggaacagcgccagcaccgtgtgtgtgtgccacatggaaatgaataggccggtccagtcagacagataccagatggaactgtgggctgagcacagacagctgtggcagggctccttcagccccagtggtaatgtgaagcccctagctccagacaacctcacactccacaccaatgtgtccgacgaatgg

SEQ ID NO: 133 - DNA, 2043 bp, Mus musculus
ctcgcaggttctggctggacttctcgaagctgaggagaagcagagggacctggcttctgattttggatctgcgtgcttgctggttctggcgcctgctggtcttgttcctgtaacccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggcccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtaaaaaaaaaaaaaaa

SEQ ID NO: 134 - DNA, 3583 bp, Mus musculus
cgcaggaaagccccgcgcggcgcgtggagcctgaactcgcaggttctggctggacttctcgaagctgaggagaagcagagggacctggcttctgattttggatctgcgtgcttgctggttctggcgcctgctggtcttgttcctgtaacctaggactcggggcttgcacatgctttttttttgaagttgctggagagggagcccaggaccttgtgcaggcaccttttgtgtccccaatggggcggctttgcaccaagttcctgacctctgtgggctgtctgattttgctgttggtgactggatctgggagcatcaaggtcctgggtgagcccacctgcttctctgactacatccgcacttccacgtgtgagtggttcctggatagcgctgtggactgcagttctcagctctgcctacactacaggctgatgttcttcgagttctctgaaaacctcacatgcatcccgaggaacagtgccagcactgtgtgtgtgtgccacatggaaatgaataggccggtccaatcagacagataccagatggaactgtgggctgagcacagacagctgtggcagggctccttcagccccagtggtaatgtgaagcccctagctccagacaacctcacactccacaccaatgtgtccgacgaatggctgctgacctggaataacctgtacccatcgaacaacttactgtacaaagacctcatctccatggtcaacatctccagagaggacaaccctgcagaattcatagtctataatgtgacctacaaggaacccaggctgagcttcccgatcaacatcctgatgtcaggggtctactatacggcgcgtgtgagggtcagatcccagatactcactggcacctggagtgagtggagtcctagcatcacgtggtacaaccacttccagctgcccctgatacagcgccttccactgggggtcaccatctcctgcctctgcatcccgttgttttgcctgttctgttacttcagcattaccaagattaagaagatatggtgggaccagattcccaccccagcacgcagtcccttggtggccatcatcattcaggatgcacaggtgcccctctgggataagcagacccgaagccaggagtcaaccaagtacccgcactggaaaacttgtctagacaagctgctgccttgcttgctgaagcacagagtaaagaagaagacagacttcccgaaggctgccccaaccaagtctctccagagtcctggaaaggcaggctggtgtcccatggaggtcagcaggaccgtcctctggccagagaatgttagtgtcagtgtggtgcgctgtatggagctgtttgaggccccagtacagaatgtggaggaggaagaagatgagatagtcaaagaggacctgagcatgtcacctgagaacagcggaggctgcggcttccaggagagccaggcagacatcatggctcggctcactgagaacctgttttccgacttgttggaggctgagaatgggggccttggccagtcagccttggcagagtcatgctcccctctgccttcaggaagtgggcaggcttctgtatcctgggcctgcctccccatggggcccagtgaggaggccacatgccaggtcacagagcagccttcacacccaggccctctttcaggcagcccagcccagagtgcacctactctggcttgcacgcaggtcccacttgtccttgcagacaatcctgcctaccggagttttagtgactgctgtagcccggccccaaatcctggagagctggctccagagcagcagcaggctgatcatctggaagaagaggagcctccaagcccggctgacccccattcttcagggccaccaatgcagccagtggagagctgggagcagatccttcacatgagtgtcctgcagcatggggcagctgctggctccaccccagcccctgccggtggctaccaggagtttgtgcaggcagtgaagcagggtgccgcccaggatcctggggtgcctggtgtcaggccttctggagaccccggttacaaggccttctcgagcctgctcagcagcaatggcatccgcggggacacagcagcagcggggactgacgatgggcatggaggctacaagcccttccagaatcctgttcctaaccagtcccctagctccgtgcccttatttactttcggactagacacggagctgtcacccagtcctctgaactcagacccacccaaaagccccccagaatgccttggtctggagctggggctcaaaggaggtgactgggtgaaggcccctcctcctgcagatcaggtgcccaagccctttggggatgacctgggctttggtattgtgtactcgtccctcacttgccacttgtgtggccacctgaagcaacaccacagccaggaggaaggtggccagagccccatcgttgctagccctggctgtggctgctgctacgatgacagatcaccatccctggggagcctctcgggggccttggaaagctgtcctgagggaataccaccagaagccaacctcatgtcagcacccaagacaccctcaaacttgtcaggggagggcaagggccctggtcactctcctgttcccagccagacgaccgaggtgcctgtgggcgccctgggcattgctgtttcttaggtgagtgagtgtgctgttgttgctgaggtctgtgctgaggccagggttcctccaagccagggaagtacttcctgggagacagcccagctggcaggtttcccagaaatccagagaatggtgaattgaagatgtaaacttggcctgaccctggacgctcggagcctggctgtctcctcttccactggcctgggctctcctccctcccaagggatacaggggctcactgtgcttggtcccacagcagtgctgacgttcctaagtcctgggctttcctagctgatgttgtcctacctactcagtcccattttgtccaccgaatagacctgtcactcaaggctctcagcggtcctgccatagctgctggacgctcccagctggaagctgggcctagaaactcacagatggcctggcagtggcatgggaggccctaaaaattagtggaaattttgagagaggacaggtattgccccacagaggccattcattgaacagccaggactgggactagaggcagagcctgctgtcctccgctcagttgtagaaagcaacaaggacacaaacttgattgcccaaagtcactgccagttacccacatatgaccagaagccagggctcctgggatgtggaagataaacaaacacagttgccgggtggcagggccccagcgggcacgataactggcagtcaaggcgatacctcgagggaactgtggggctggtcctggttggtggtcaggtggtagggatagcagatggcagactttggtgagtgagtgagtctgactgtgttctggaagatgggaccgggctcagcactgtctgctcacgtccccactgttgcaacacctagtctgtttgcaaggaggacaggacaggtcacatggagctttatgtcaataaagtctttatcttgtc

SEQ ID NO: 135 - DNA, 3697 bp, Mus musculus; identical to SEQ ID NO: 1

SEQ ID NO: 136 - DNA, 3697 bp, Mus musculus; identical to SEQ ID NO: 1

SEQ ID NO: 137 - DNA, 21 bp, Artificial Sequence
  Primer
caacggatttggtcgtattgg

SEQ ID NO: 138 - DNA, 26 bp, Artificial Sequence
  Primer
ggcaacaatatccactttaccagagt

SEQ ID NO: 139 - DNA, 21 bp, Artificial Sequence
  Probe
cgcctggtcaccagggctgct

SEQ ID NO: 140 - DNA, 19 bp, Artificial Sequence
  Primer
gaaggtgaaggtcggagtc

SEQ ID NO: 141 - DNA, 20 bp, Artificial Sequence
  Primer
gaagatggtgatgggatttc

SEQ ID NO: 142 - DNA, 20 bp, Artificial Sequence
  Probe
caagcttcccgttctcagcc

SEQ ID NO: 143 - DNA, 20 bp, Artificial Sequence
  Probe
tggaatcatattggaacatg

SEQ ID NO: 144 - DNA, 20 bp, Artificial Sequence
  Primer
ggcaaattcaacggcacagt

SEQ ID NO: 145 - DNA, 20 bp, Artificial Sequence
  Primer
gggtctcgctcctggaagat

SEQ ID NO: 146 - DNA, 27 bp, Artificial Sequence
  Probe
aaggccgagaatgggaagcttgtcatc

SEQ ID NO: 147 - DNA, 23 bp, Artificial Sequence
  Primer
tgttctagagacagccgcatctt

SEQ ID NO: 148 - DNA, 21 bp, Artificial Sequence
  Primer
caccgaccttcaccatcttgt

SEQ ID NO: 149 - DNA, 24 bp, Artificial Sequence
  Probe
ttgtgcagtgccagcctcgtctca

SEQ ID NO: 150 - DNA, 19 bp, Artificial Sequence
  Primer
aatggtcccaccaattgca

SEQ ID NO: 151 - DNA, 23 bp, Artificial Sequence
  Primer
ctccgttgttctcagggatacac

SEQ ID NO: 152 - DNA, 21 bp, Artificial Sequence
  Probe
tttttctgctctccgaagccc

SEQ ID NO: 153 - DNA, 21 bp, Artificial Sequence
  Primer
tcccattttgtccaccgaata

SEQ ID NO: 154 - DNA, 20 bp, Artificial Sequence
  Primer
gtttctaggcccagcttcca

SEQ ID NO: 155 - DNA, 25 bp, Artificial Sequence
  Probe
tgtcactcaaggctctcagcggtcc

SEQ ID NO: 156 - RNA, 19 nt, Artificial Sequence
  Antisense Compound
cgagaggcggacgggaccg

SEQ ID NO: 157 - DNA, 21 bp, Artificial Sequence
  Antisense Compound
  misc_feature (1)...(19): Bases at these positions are RNA
cgagaggcggacgggaccgtt

SEQ ID NO: 158 - DNA, 21 bp, Artificial Sequence
  Antisense Compound
  misc_feature (3)...(21): Bases at these positions are DNA
ttgcucuccgccugcccuggc

SEQ ID NO: 159 - RNA, 19 nt, Artificial Sequence
  Antisense Compound
gcucuccgccugcccuggc